(19) United States
(12) Patent Application Publication
(10) Pub. No.: US 2008/0200491 A1
(43) Pub. Date: Aug. 21, 2008

(54) 1,2-Dihydro-Spiro[3H-Indole-3,4'-Piperidine] Compounds, as Modulators of the Mas Receptor Novel

(76) Inventors: Thuy-Anh Tran, San Diego, CA (US); Sangdon Han, San Diego, CA (US); P. Douglas Boatman, San Diego, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/917,731

(22) PCT Filed: Jun. 21, 2006
(86) PCT No.: PCT/US06/24020

§ 371 Date: Apr. 9, 2008

Related U.S. Application Data
(60) Provisional application No. 60/692,716, filed on Jun. 22, 2005.

(51) Int. Cl. A61K 31/438; C07D 471/10; A61P 9/10; A61P 25/02; A61P 25/16

(52) U.S. Cl. 514/278; 546/17

(57) ABSTRACT

The present invention relates to certain 1,2-dihydro-spiro[3H-indole-3,4′-piperidine]compounds of Formula (Ia): and pharmaceutically acceptable salts, solvates and hydrates thereof, wherein G, R1, R2, R3, R4, R5, and Ar are as disclosed herein (“Compound(s) of the Invention”), which are useful, for example, as cardio-protective and/or neuro-protective agents. The invention also provides pharmaceutical compositions comprising a Compound of the Invention and methods for treating, preventing and/or managing a vascular, cardiovascular or neurological disease or disorder, comprising administering to a patient in need thereof a Compound of the Invention.

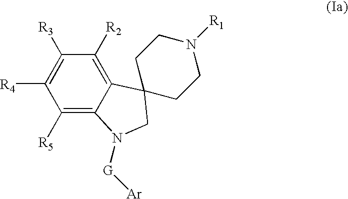

### FIELD OF THE INVENTION

The present invention relates to certain novel 1,2-Dihydro-spiro[3H-indole-3,4′-piperidine] compounds and pharmaceutically acceptable salts, free bases, solvates, hydrates, stereoisomers, or prodrugs thereof, which are useful, for example, as cardio-protective or neuro-protective agents in mammals. The invention encompasses compositions comprising a 1,2-Dihydro-spiro[3H-indole-3,4′-piperidine] Compound and methods for treating or preventing a disease or disorder comprising the administration of a 1,2-Dihydro-spiro[3H-indole-3,4′-piperidine] Compound to a patient in need thereof. Such a disease or disorder includes, for example, a vascular or cardiovascular disease or disorder such as atherosclerosis, reperfusion injury, acute myocardial infarction, high blood pressure, primary or secondary hypertension, renal vascular hypertension, acute or chronic congestive heart failure, left ventricular hypertrophy, vascular hypertrophy, glaucoma, primary or secondary hyperaldosteronism, diabetic neuropathy, glomerulonephritis, scleroderma, glomerular sclerosis, renal failure, renal transplant therapy, diabetic retinopathy, migraine, and neurological diseases or disorders such as diabetic peripheral neuropathy, pain, stroke, cerebral ischemia and Parlinson's disease. The invention also relates to a modulator of the Mas G-protein coupled receptor including, for example, a 1,2-Dihydro-spiro[3H-indole-3,4′-piperidine] Compound as disclosed herein.

### BACKGROUND OF THE INVENTION

G protein-coupled receptors (GPCRs) share the common structural motif of having seven sequences of between 22 to 24 hydrophobic amino acids that form seven alpha helices, each of which spans the cell membrane. The transmembrane helices are joined by strands of amino acids having a larger loop between the fourth and fifth transmembrane helix on the extracellular side of the membrane. Another larger loop, composed primarily of hydrophilic amino acids, joins transmembrane helices five and six on the intracellular side of the membrane. The carboxy terminus of the receptor lies intracellularly with the amino terminus residing in the extracellular space. It is thought that the loop joining helices five and six, as well as the carboxy terminus, interact with the G protein. Currently, the G proteins that have been identified are Gq, Gs, Gi, and Go.

Under physiological conditions, GPCRs exist in the cell membrane in equilibrium between two different states or conformations: an “inactive” state and an “active” state. A receptor in an inactive state is unable to link to the intracellular transduction pathway to produce a biological response. Change of the receptor conformation to the active state allows linkage to the transduction pathway and produces a biological response. Physiologically, these conformational changes are induced in response to binding of a molecule to the receptor. Several types of biological molecules can bind to specific receptors, such as peptides, hormones or lipids, and can cause a cellular response. Modulation of particular cellular responses can be extremely useful for the treatment of disease states, and a number of chemical agents that act on GPCRs are useful for the treatment of disease.

The Mas protooncogene encodes a GPCR protein (Mas) and was first detected in vivo by its tumorogenic properties which originate from rearrangement of its 5′ flanking region (Young, D. et al., Cell 45:711-719 (1996)). Subsequent studies have indicated that the tumorogenic properties of Mas appear to be negligible. The lack of an identified activating ligand for the Mas receptor has made definition of its biological role difficult.

Originally, the angiotensin II (Ang II) peptide was thought to be a ligand for the Mas receptor (Jackson et al., Nature 335:437-440 (1988)). However, it was subsequently determined that intracellular calcium responses in Mas receptor-transfected cells only occurred in cells that already express an Ang II receptor (Ambroz et al. Biochem. Biophys. Acta 1133:107-111 (1991)). Other experiments demonstrated a possible role for Mas receptor in modulating intracellular signaling of an Ang II receptor after Ang II stimulation (von Bohlen und Halbech et al., J. Neurophysiol. 83:2012-2020 (2000)). In addition, Dong et al. reported that the Mas receptor did not bind to angiotensins I and II, but the Mas receptor did bind to a peptide called NPFF, although fairly wealdy (EC50 about 400 nM) (Dong et al., Cell 106:619-632 (2001)). A recent report that the biologically relevant angiotensin fragment Ang (1-7) (H-Asp-Arg-Val-Tyr-Ile-His-Pro-OH) is a high affinity ligand for the Mas receptor (Kd=0.33 nM) (Santos, R. A. S. et al., PNAS 100:8258-8263 (2003)) may point to a possible role for the Mas receptor in blood pressure regulation and thrombus production.

The renin/angiotensin system is one of the major pathways by which blood pressure is regulated. Renin is produced in the kidneys in response to a decrease in renal perfusion pressure when catecholamines or angiotensin II are present, or when sodium or chloride ion concentrations in the blood decline. Renin catalyzes the conversion of angiotensinogen to its inactive metabolite, angiotensin I. Angiotensin converting enzyme catalyzes the conversion of angiotensin I to angiotensin II, a powerful vasoconstrictor which acts on the angiotensin II receptor. The cardiovascular and baroreflex actions of Ang (1-7) are reported to counteract those of angiotensin II. Whereas, angiotensin II, acting at the AT2 receptor causes vasoconstriction and concurrent increase in blood pressure, Ang (1-7) acting at the Mas receptor has been reported to cause vasodilation and blood pressure decrease (Santos, R. A. et al., Regul. Pept. 91:45-62 (2000)).

The standard treatment for myocardial infarction is reperfusion of the ischemic area by thrombolysis or percutaneous coronary angioplasty. Release of the blockage and return of blood flow to the affected area is crucial for heart tissue survival; however, damage beyond that generated by ischemia is typically observed in the reperfused heart tissue. The manifestations of reperfusion injury include arrhythmia, reversible contractile dysfunction-myocardial stunning, endothelial dysfunction and cell death. Currently, there is no effective treatment for reperfusion injury available. Ang (1-7) has been shown to improve post-ischemic myocardial function in an ischemia/reperfusion model using isolated rat hearts. (Ferreira, A. J. et al., Braz. J. of Med. and Biol. Res. 35(9):1083-1090 (2002)).

In addition to the immediate adverse effects of myocardial infarction, subsequent loss of contractile function, scarring and tissue remodeling often lead to congestive heart failure (CHF). A follow-up to the Framingham Heart Study indicates that 22% of male and 46% of female myocardial infarction victims will be disabled with CHF within six years following their heart attack. Despite significant advances in the treatment and prevention of congestive heart disease, the prognosis for patients with CHF remains poor. A recent study reported that 12% of patients die within three months of diagnosis, 33% die within one year and approximately 60% die within five years.

Hypertension is the most common factor contributing to CHF. The American Heart Association estimates that 75% of CHF cases have antecedent hypertension. In most hypertensive individuals, cardiac output is normal but there is an increase in resistance in the arteriole circulation causing the heart to pump harder to overcome the peripheral resistance and perfuse the peripheral tissues. The left ventricle develops pressure hypertrophy, which leads to myocardial remodeling and reduced pumping capacity resulting in a cycle of reduced cardiac function. Control of blood pressure is an effective treatment for chronic CHF and considerable effort has been focused on the development of therapies for hypertension. Foremost among these, are the angiotensin converting enzyme inhibitors (ACEIs). ACEIs block the conversion of angiotensin I to angiotensin II, thus, decreasing the hypertensive effects resulting from angiotensin II. Additionally, beta blockers, which act on the beta adrenergic receptor and inhibit sympathetic innervation of the heart, are used to treat chronic hypertension. Although these therapies are effective, there can be severe side effects associated with their use. As such, they are not tolerated by all individuals and there is a need for new and effective alternatives to these therapies.

Ang (1-7) has been shown to have a vasodilatory effect in many vascular beds, including canine and porcine coronary arteries, rat aorta, and feline mesenteric arteries. Chronic infusion of Ang (1-7) in spontaneously hypertensive rats and Dahl salt-sensitive rats has been shown to reduce mean arterial blood pressure. Ang (1-7) has been shown to block the Ang II induced vasoconstriction in isolated human arteries and antagonized vasoconstriction in forearm circulation by Ang II in normotensive men. Direct vasodilation to the same extent in basal forearm circulation of both normotensive and hypertensive patients by Ang (1-7) has been observed. Additionally, although the mechanism is undefined, it is believed that the vasodilation effects of bradykinin are potentiated by Ang (1-7).

The discovery that Ang (1-7) is an endogenous ligand for the Mas receptor has provided validation of the importance of the development of therapeutic entities which modulate Mas receptor activity. However, the inherent instability of Ang (1-7) and the likelihood that it is not absorbed upon oral administration make it ineffective as a therapeutic agent. These considerations highlight the importance of the development of pharmacologically useful modulators of the Mas receptor for the safe and effective treatment and/or prevention of human disease.

Citation of any reference throughout this application is not to be construed as an admission that such reference is prior art to the present application.

### SUMMARY OF THE INVENTION

Applicants have generated certain 1,2-dihydro-spiro[3H-indole-3,4′-piperidine] compounds and pharmaceutically acceptable salts, free bases, solvates, hydrates, stereoisomer, and prodrugs thereof, which are useful, for example, as cardio-protective or neuro-protective agents in mammals.

While the literature cited above may indicate that an agonist of the Mas receptor would be cardio-protective and decrease blood pressure, Applicants have unexpectedly identified compounds that can act as inverse agonists of the Mas receptor which are cardio-protective and do not raise blood pressure. For example, Applicants have identified 1′-(allyl)-1,2-dihydro-5-fluoro-1-(2,3-difluoro-benzoyl)-spiro[3H-indole-3,4′-piperidine], referred to herein as Compound S75 (see Table 1 below), and found that this compound can act as an inverse agonist of the Mas receptor (see Example 2 and FIG. 1), is cardio-protective (see Example 4 and FIGS. 2-4), does not raise blood pressure (see Example 5 and FIG. 5). In fact, Compound S75 selectively inhibits angiotensin-stimulated hypertension (see Example 6 and FIG. 6).

TABLE 1

| Cmpd No. | Chemical Structure | Chemical Name |
|---|---|---|
| S75 | 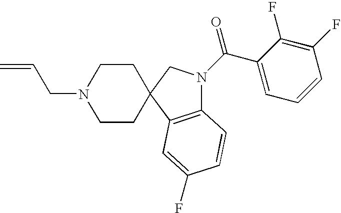 | 1′-(allyl)-1,2-dihydro-5-fluoro-1-(2,3-difluoro-benzoyl)-spiro[3H-indole-3,4′-piperidine] |

The Mas receptor is a GPCR that couples to the Gq G-protein. Although some lines of evidence point to Ang (1-7) as a ligand for the Mas receptor (see Santos et al., supra, 2003), Applicants have advantageously chosen herein an assay that does not rely on using a ligand for the Mas receptor. Thus, this assay is not biased by the use of a particular ligand for the Mas receptor. Applicants have over-expressed the Mas receptor in cells such that the receptor is constitutively active in the absence of a ligand. Applicants have used an IP3 assay to screen for compounds that decrease the amount of Mas receptor functionality and disclose herein several compounds that can significantly decrease Mas receptor functionality. The compounds can act as inverse agonists at a Mas receptor. An “inverse agonist” means a compound that binds to a receptor so as to reduce the baseline intracellular response of the receptor observed in the absence of agonist.

While the Compounds of the Invention have activity at the Mas receptor, it is understood that a Compound of the Invention may also act at another receptor or receptors which can elicit some of the biological properties of the compound such as, for example, effects on blood pressure, cardio-protection, or neuro-protection. For example, several genes related to the Mas receptor gene, called Mas-related genes or mrgs, are known in the art (Dong et al. supra, 2001). Also, as mentioned above, a peptide called NPFF has been found to bind to the Mas receptor, although weakly (Dong et al. supra, 2001). The NPFF peptide has been implicated in pain response and is also reported to have effects on the cardiovascular system (Allard et al. J. Pharmacol Exp. Ther. 274:577-583 (1995); Laguzzi et al., Brain Res. 711:193-202 (1996)). The NPFF peptide binds with high affinity to two neuropeptide-Y like GPCRs called NPFF1 (Kd=1.3 nM) and NPFF2 (Kd=0.3 nM) (Bonini et al., J. Biol. Chem. 275:39324-39331 (2000); Elshourbagy et al., J. Biol. Chem., 275:25965-25971 (2000)).

The present invention encompasses 1,2-dihydro-spiro[3H-indole-3,4′-piperidine] compounds of Formula (Ia):

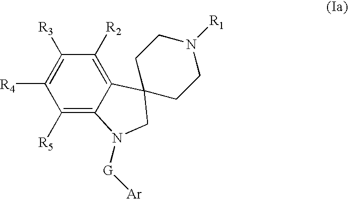

or a pharmaceutically acceptable salt, solvate or hydrate thereof;

wherein:

G is C(═O) or S(═O)₂;

R₁ is n-propyl optionally substituted with 1, 2, 3, 4, 5, 6, or 7 fluorine atoms;

R₂, R₃, R₄, and R₅ are each selected independently from the group consisting of H, C₁₋₆ acyl, C₁₋₆ acyloxy, C₂₋₆ alkenyl, C₁₋₆ alkoxy, C₁₋₆ alkyl, C₁₋₆ alkylamino, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylthiocarboxamide, C₁₋₆ alkylthioureyl, C₁₋₆ alkylureyl, amino, di-C₁₋₆-alkylamino, C₁₋₆ alkoxycarbonyl, carboxamide, carboxy, cyano, C₃₋₆ cycloalkyl, di-C₁₋₆-alkylcarboxamide, di-C₁₋₆-alkylsulfonamide, di-C₁₋₆-alkylthiocarboxamido, C₁₋₆ haloalkoxy, C₁₋₆ haloalkyl, halogen, C₁₋₆ haloalkylsulfinyl, C₁₋₆ haloalkylsulfonyl, C₁₋₆ haloalkylthio, heterocyclic, hydroxyl, nitro, sulfonamide, and thiol; and

Ar is aryl or heteroaryl each optionally substituted with 1, 2, 3, 4, 5, 6, or 7 substituents selected independently from the group consisting of C₁₋₆ acyl, C₁₋₆ acyloxy, C₂₋₆ alkenyl, C₁₋₆ alkoxy, C₁₋₆ alkyl, C₁₋₆ alkylamino, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylthiocarboxamide, C₁₋₆ alkylthioureyl, C₁₋₆ alkylureyl, amino, di-C₁₋₆-alkylamino, C₁₋₆ alkoxycarbonyl, carboxamide, carboxy, cyano, C₃₋₆ cycloalkyl, di-C₁₋₆-alkylcarboxamide, di-C₁₋₆-alkylsulfonamide, di-C₁₋₆-alkylthiocarboxamido, C₁₋₆ haloalkoxy, C₁₋₆ haloalkyl, halogen, C₁₋₆ haloalkylsulfinyl, C₁₋₆ haloalkylsulfonyl, C₁₋₆ haloalkylthio, heterocyclic, hydroxyl, nitro, sulfonamide, and thiol;

provided that the compound is not of the group consisting of:
1′-(propyl)-1,2-dihydro-5,7-dimethyl-1-(2-chloro-benzenesulfonyl)-spiro[3H-indole-3,4′-piperidine];
1′-(propyl)-1,2-dihydro-5-methyl-1-(2,3-difluoro-benzoyl)-spiro[3H-indole-3,4′-piperidine]; and
1′-(propyl)-1,2-dihydro-5-methyl-1-(2,6-difluoro-benzoyl)-spiro[3H-indole-3,4′-piperidine].

The compounds of Formula (Ia) are further described below.

Compounds of the Invention can also include all isotopes of atoms occurring in the intermediates or final compounds. Isotopes include those atoms having the same atomic number but different mass numbers. For example, isotopes of hydrogen include tritium and deuterium. Other examples include, but not limited to, ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I atoms.

The 1,2-dihydro-spiro[3H-indole-3,4′-piperidine] compounds of Formula (Ia), including pharmaceutically acceptable salts, free bases, solvates, hydrates, stereoisomers and prodrugs thereof (“Compound(s) of the Invention”), are useful as a cardio-protective and/or neuroprotective agents.

In one embodiment, a Compound of the Invention does not significantly increase blood pressure. The Compounds of the Invention are also useful for treating, preventing and/or managing vascular or cardiovascular diseases or disorders including, but not limited to, atherosclerosis, reperfusion injury, acute myocardial infarction, high blood pressure, hypertension, primary or secondary hypertension, renal vascular hypertension, acute or chronic congestive heart failure, left ventricular hypertrophy, vascular hypertrophy, glaucoma, primary or secondary hyperaldosteronism, diabetic neuropathy, glomerulonephritis, scleroderma, glomerular sclerosis, renal failure, renal transplant therapy, diabetic retinopathy, other vascular diseases or disorders and migraines. A Compound of the Invention is also useful for treating, preventing and/or managing neurological diseases or disorders including, but not limited to, diabetic peripheral neuropathy, pain, stroke, cerebral ischemia and Parkinson's disease in a patient in need thereof. The Compounds of the Invention can also be used in patients at risk of such diseases and disorders as cardio-protective or neuro-protective agents.

One aspect of the present invention pertains to methods for treating or preventing a vascular or cardiovascular disease or disorder comprising administering to a patient in need thereof an effective amount of a Compound of the Invention or a pharmaceutical composition thereof. In some embodiments, the compound of the present invention does not significantly increase blood pressure. In some embodiments, the vascular or cardiovascular disorder is atherosclerosis, reperfusion injury, acute myocardial infarction, high blood pressure, primary or secondary hypertension, renal vascular hypertension, acute or chronic congestive heart failure, left ventricular hypertrophy, vascular hypertrophy, glaucoma, primary or secondary hyperaldosteronism, diabetic nephropathy, glomerulonephritis, scleroderma, glomerular sclerosis, renal failure, renal transplant therapy, diabetic retinopathy or migraine.

One aspect of the present invention pertains to methods for treating or preventing a neurological disease or disorder comprising administering to a patient in need thereof an effective amount of a Compound of the Invention or a pharmaceutical composition thereof. In some embodiments, the neurological disease or disorder is diabetic peripheral neuropathy, pain, stroke, cerebral ischemia or Parkinson's disease.

One aspect of the present invention pertains to methods for treating or preventing a disease or disorder treatable or preventable by inhibiting Mas receptor function, comprising administering to a patient in need thereof an effective amount of a Compound of the Invention or a pharmaceutical composition thereof. In some embodiments, the disease or disorder is a vascular or cardiovascular disease or disorder. In some embodiments, the vascular or cardiovascular disease or disorder is atherosclerosis, reperfusion injury, acute myocardial infarction, high blood pressure, primary or secondary hypertension, renal vascular hypertension, acute or chronic congestive heart failure, left ventricular hypertrophy, vascular hypertrophy, glaucoma, primary or secondary hyperaldosteronism, diabetic nephropathy, glomerulonephritis, scleroderma, glomerular sclerosis, renal failure, renal transplant therapy, diabetic retinopathy or migraine. In some embodiments, the disease or disorder is a neurological disease or disorder. In some embodiments, the neurological disease or disorder is diabetic peripheral neuropathy, pain, stroke, cerebral ischemia or Parkinson's disease.

One aspect of the present invention pertains to use of a compound of the present invention for the manufacture of a medicament comprising a Compound of the Invention for use in the treatment of a vascular or cardiovascular disease.

One aspect of the present invention pertains to use of a compound of the present invention for the manufacture of a medicament comprising a Compound of the Invention for use in the treatment of a neurological disease.

One aspect of the present invention pertains to use of a compound of the present invention for the manufacture of a medicament comprising a Compound of the Invention for use as a neuro-protective agent.

One aspect of the present invention pertains to use of a compound of the present invention for the manufacture of a medicament comprising a Compound of the Invention for use as a cardio-protective agent.

In a particular embodiment, a medicament comprising a Compound of the Invention is useful for treating, preventing and/or managing a vascular or cardiovascular disorder and/or a neurological disorder.

One aspect of the present invention pertains to use of a compound of the present invention for use in a method of treatment of the human or mammal body by therapy.

One aspect of the present invention pertains to use of a compound of the present invention for use in a method of treatment of a vascular or cardiovascular disease of the human or mammal body by therapy.

One aspect of the present invention pertains to use of a compound of the present invention for use in a method of treatment of a neurological disease or disorder of the human or mammal body by therapy.

One aspect of the present invention pertains to methods for producing a pharmaceutical composition comprising admixing one or more Compound(s) of the Invention and a pharmaceutically acceptable vehicle or excipient.

In one embodiment, a Compound of the Invention is used in combination with other compounds for the treatment of a vascular, cardiovascular or neurological disease or disorder. For example, in one embodiment, a Compound of the Invention is used in combination with, or in place of, angiotensin-converting enzyme (ACE) inhibitors to treat the diseases or disorders for which such ACE inhibitors are conventionally used.

One aspect of the present invention pertains to methods for treating or preventing a disorder treatable or preventable by inhibiting Mas receptor function, comprising administering to a patient in need thereof an effective amount of a Compound of the Invention. In one embodiment, the disorder is a vascular or cardiovascular disease or disorder and in another embodiment, the disorder is a neurological disease or disorder.

One aspect of the present invention pertains to methods for inhibiting Mas receptor function in a cell, comprising contacting a cell capable of expressing the Mas receptor with an effective amount of a Compound of the Invention.

One aspect of the present invention pertains to pharmaceutical compositions comprising a Compound of the Invention and a pharmaceutically acceptable vehicle or excipient. The compositions are useful as cardio-protective and/or neuro-protective agents and for treating or preventing a vascular or cardiovascular disorder and/or a neurological disorder in a patient.

One aspect of the present invention pertains to methods for treating a vascular or cardiovascular disorder and/or a neurological disorder, comprising administering to a patient in need thereof a Compound of the Invention.

One aspect of the present invention pertains to methods for preventing a vascular or cardiovascular disorder and/or a neurological disorder, comprising administering to a patient in need thereof a Compound of the Invention.

One aspect of the present invention pertains to methods for managing a vascular or cardiovascular disorder and/or a neurological disorder, comprising administering to a patient in need thereof a Compound of the Invention.

One aspect of the present invention pertains to methods for treating a neurological disease or disorder comprising administering to a patient in need thereof an effective amount of a Compound of the Invention or a pharmaceutical composition thereof.

One aspect of the present invention pertains to methods for preventing a neurological disease or disorder comprising administering to a patient in need thereof an effective amount of a Compound of the Invention or a pharmaceutical composition thereof.

One aspect of the present invention pertains to methods for managing or preventing a neurological disease or disorder comprising administering to a patient in need thereof an effective amount of a Compound of the Invention or a pharmaceutical composition thereof.

The invention also relates to a method for selectively inhibiting Mas receptor activity in a human host, comprising administering a Compound of the Invention that selectively inhibits activity of the Mas receptor gene product to a human host in need of such selective inhibition.

The invention still further relates to a kit comprising a container containing a Compound of the Invention. The kit may further comprise printed instructions for using the Compound of the Invention to treat, prevent and/or manage any of the aforementioned diseases or disorders.

The present invention may be understood more fully by reference to the following detailed description and illustrative examples, which are intended to exemplify non-limiting embodiments of the invention.

### BRIEF DESCRIPTION OF THE DRAWINGS

FIG. 1 shows an IP3 assay of Compound S75 using HEK293 cells that over-express the human Mas receptor resulting in constitutive activity of the Mas receptor in these cells.

FIG. 2 shows the results of another ischemia-reperfusion assay in isolated rat hearts treated with Compound S75 or vehicle (control).

FIG. 3 shows end diastolic pressure (EDP) readings in the isolated rat hearts from the ischemia-reperfusion assay shown in FIG. 2.

FIG. 4 shows epicardial electrogram recordings in selected isolated rat hearts from the ischemia-reperfusion assay shown in FIG. 3.

FIG. 5 shows blood pressure measurements in rats treated with Compound S75, vehicle, or control compounds angiotensin II (AngII) and sodium nitroprusside (SNP).

FIG. 6 shows selective inhibition on angiotensin II induced vasoconstriction in isolated rat aortic rings with Compound S75.

### DETAILED DESCRIPTION OF THE INVENTION

The present invention is drawn to compounds which bind to and modulate the activity of a GPCR, referred to herein as Mas, and uses thereof. The term Mas as used herein includes the human sequences found in GeneBank Accession No. CR542261, naturally-occurring allelic variants, mammalian orthologs, and recombinant mutants thereof.

The present invention encompasses 1,2-dihydro-spiro[3H-indole-3,4′-piperidine] compounds of Formula (Ia):

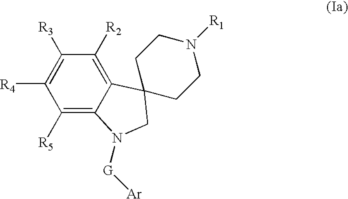

and pharmaceutically acceptable salts, free bases, solvates, hydrates, or stereoisomers thereof, wherein G, R₁, R₂, R₃, R₄, R₅, and Ar are as defined herein, infra and supra, provided that the compound is not one of the following three compounds:

TABLE 2

| Chemical Structure | Chemical Name |
|---|---|
| 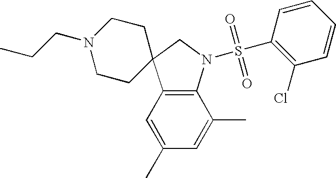 | 1′-(propyl)-1,2-dihydro-5,7-dimethyl-1-(2-chloro-benzenesulfonyl)-spiro[3H-indole-3,4′-piperidine] |
| 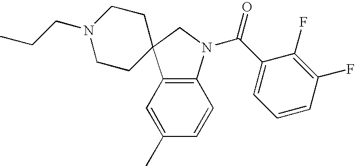 | 1′-(propyl)-1,2-dihydro-5-methyl-1-(2,3-difluoro-benzoyl)-spiro[3H-indole-3,4′-piperidine] |
| 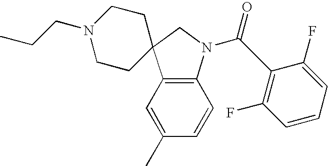 | 1′-(propyl)-1,2-dihydro-5-methyl-1-(2,6-difluoro-benzoyl)-spiro[3H-indole-3,4′-piperidine] |

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

As used herein, “substituted” indicates that at least one hydrogen atom of the chemical group is replaced by a non-hydrogen substituent or group. When a chemical group herein is “substituted” it may have up to the full valance of substitution; for example, a methyl group can be substituted by 1, 2, or 3 substituents, a methylene group can be substituted by 1 or 2 substituents, a phenyl group can be substituted by 1, 2, 3, 4, or 5 substituents, a naphthyl group can be substituted by 1, 2, 3, 4, 5, 6, or 7 substituents and the like. Likewise, “substituted with one or more substituents” refers to the substitution of a group with one substituent up to the total number of substituents physically allowed by the group. Further, when a group is substituted with more than one group they can be identical or they can be different.

In some embodiments, G is C(═O).

In some embodiments, compounds of the present invention are represented by Formula (Ic) as shown below:

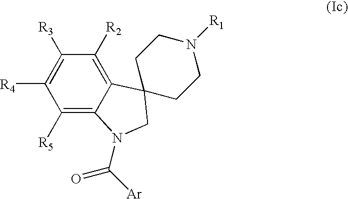

wherein each variable in Formula (Ic) are as defined herein infra and supra.

In some embodiments, G is S(═O)₂.

In some embodiments, compounds of the present invention are represented by Formula (Ie) as shown below:

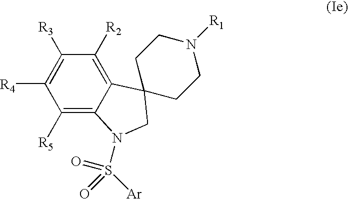

wherein each variable in Formula (Ie) are as defined herein infra and supra.

In some embodiments, R₁ is n-propyl.

In some embodiments, compounds of the present invention are represented by Formula (Ig) as shown below:

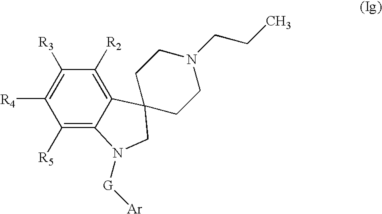

wherein each variable in Formula (Ig) are as defined herein infra and supra.

In some embodiments, R₁ is n-propyl substituted with 1, 2, 3, 4, 5, 6, or 7 fluorine atoms.

In some embodiments, R₁ is selected from the group consisting of —CH₂CH₂CF₃, —CH₂CF₂CF₃, —CH₂CH₂CHF₂, —CH₂CF₂CHF₂, and —CH₂CF₂CH₃.

In some embodiments, R₁ is —CH₂CH₂CF₃.

In some embodiments, R₂, R₃, R₄, and R₅ are each selected independently from the group consisting of H, C₁₋₆ alkoxy, C₁₋₆ alkyl, C₁₋₆ alkylcarboxamide, C₁₋₆ alkylsulfonamide, C₁₋₆ alkylsulfinyl, C₁₋₆ alkylsulfonyl, C₁₋₆ alkylthio, carboxamide, cyano, C₃₋₆ cycloalkyl, di-C₁₋₆-alkylcarboxamide, C₁₋₆ haloalkoxy, C₁₋₆ haloalkyl, halogen, C₁₋₆ haloalkylsulfinyl, C₁₋₆ haloalkylsulfonyl, C₁₋₆ haloalkylthio, hydroxyl, and sulfonamide.

In some embodiments, R₂, R₃, R₄, and R₅ are each selected independently from the group consisting of H, —OCH₃, —CH₃, —CH₂CH₃, —CH(CH₃)₂, —C(CH₃)₃, —S(═O)₂NH₂, —S(═O)₂NHCH₃, —S(═O)CH₃, —S(═O)₂CH₃, —SCH₃, —C(═O)NH₂, —C N, —C(═O)N(CH₃)₂, —OCF₃, —CF₃, —CF₂CF₃, F, Cl, Br, I, —S(═O)CF₃, —S(═O)₂CF₃, —SCF₃, and —OH.

In some embodiments, R₂, R₄, and R₅ are each independently H, —CH₃, or F; and R₃ is selected from the group consisting of H, —OCH₃, —CH₂CH₃, —CH(CH₃)₂, —C(CH₃)₃, —S(═O)₂NH₂, —S(═O)₂NHCH₃, —S(═O)CH₃, —S(═O)₂CH₃, —SCH₃, —C(═O)NH₂, —C≡N, —C(═O)N(CH₃)₂, —OCF₃, —CF₃, —CF₂CF₃, F, Cl, Br, I, —S(═O)CF₃, —S(═O)₂CF₃, —SCF₃, and —OH.

In some embodiments, R₂, R₄, and R₅ are each H; and R₃ is selected from the group consisting of H, —OCH₃, —CH(CH₃)₂, —C(CH₃)₃, —S(═O)₂NH₂, —S(═O)₂CH₃, —C(═O)NH₂, —C≡N, —C(═O)N(CH₃)₂, —CF₃, F, and Cl.

In some embodiments, R₂, R₄, and R₅ are each H; and R₃ is —CH(CH₃)₂, —C(CH₃)₃, CF₃, or Cl.

In some embodiments, when R₁ is propyl and R₂, R₄, and R₅ are each H, then R₃ is a group other than CH₃.

In some embodiments, when R₁ is propyl, R₂ and R₄ are both H, and R₅ is CH₃, then R₃ is a group other than CH₃.

In some embodiments, when R₁ is propyl, G is S(═O)₂, and Ar is 2-chlorophenyl, then R₅ is a group other than CH₃.

In some embodiments, Ar is phenyl or naphthyl each optionally substituted with 1, 2, 3, 4, 5, 6, or 7 substituents selected independently from the group consisting of halo, C₁₋₆ alkoxy, C₁₋₆ alkylsulfonyl, nitro, C₁₋₆ alkyl, carboxamide, cyano, C₁₋₆ haloalkyl, C₁₋₆ haloalkoxy, and sulfonamide.

In some embodiments, Ar is phenyl or naphthyl each optionally substituted with 1, 2, 3, 4, 5, 6, or 7 substituents selected independently from the group consisting of F, Cl, Br, —OCH₃, —OCH₂CH₃, —OCH(CH₃)₂, —NO₂, —CH₃, —S(═O)₂CH₃, —S(═O)₂NH₂, —C≡N, —CF₃, and —OCF₃.

In some embodiments, Ar is selected from the group consisting of 2-chlorophenyl, 4-methoxyphenyl, 4-nitrophenyl, 3,4-difluorophenyl, 2-fluorophenyl, 4-chlorophenyl, 3-dichloromethylphenyl, 3,5-bis-trifluoromethylphenyl, 2,4-difluorophenyl, 2-methoxyphenyl, 3,5-dimethoxyphenyl, 3,4,5-trimethoxyphenyl, 3-fluorophenyl, 4-fluorophenyl, 2,3-difluorophenyl, 2,5-difluorophenyl, 3-trifluoromethyl-4-fluorophenyl, 3-trifluoromethylphenyl, phenyl, 3-nitrophenyl, 3-nitro-4-methylphenyl, naphthalen-1-yl, naphthalen-2-yl, 4-trifluoromethylphenyl, 3-chlorophenyl, 3,5-dichlorophenyl, 2,4-dichlorophenyl, 2,6-difluorophenyl, 2,6-dichlorophenyl, 2,6-bis-trifluoromethylphenyl, 2-chloro-6-fluoro-phenyl, and 2,3-dichlorophenyl.

In some embodiments, Ar is an optionally substituted 5-membered heteroaryl selected from the group consisting of the following formulae:

TABLE 3

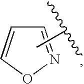

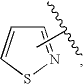

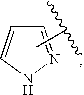

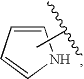

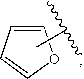

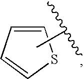

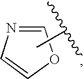

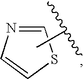

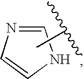

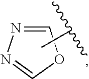

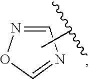

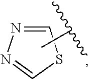

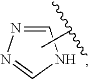

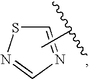

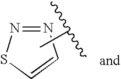

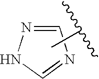

wherein the 5-member heteroaryl is bonded at any available position of the ring, for example, a imidazolyl ring can be bonded at one of the ring nitrogens (i.e., imidazol-1-yl group) or at one of the ring carbons (i.e., imidazol-2-yl, imidazol-4-yl or imiadazol-5-yl group).

In some embodiments, Ar is an optionally substituted 6-membered heteroaryl selected from the group consisting of the following formulae:

TABLE 4

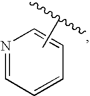

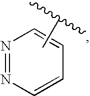

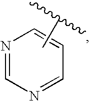

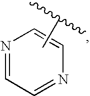

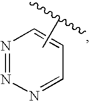

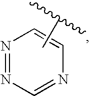

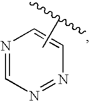

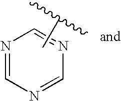

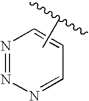

wherein the heteroaryl group is bonded at any ring carbon.

In some embodiments, Ar is heteroaryl optionally substituted with 1, 2, 3, 4, or 5 substituents selected independently from the group consisting of halo, C₁₋₆ alkoxy, C₁₋₆ alkylsulfonyl, nitro, C₁₋₆ alkyl, carboxamide, cyano, C₁₋₆ haloalkyl, C₁₋₆ haloalkoxy, and sulfonamide.

In some embodiments, Ar is thienyl, pyridinyl, furanyl, pyrazolyl, benzofuranyl, benzothienyl, or naphthyridinyl each optionally substituted with 1, 2, 3, 4, or 5 substituents selected independently from the group consisting of halo, C₁₋₆ alkoxy, nitro, C₁₋₆ alkyl, and C₁₋₆ haloalkyl.

In some embodiments, Ar is thienyl, pyridinyl, furanyl, pyrazolyl, benzofuranyl, benzothiophenyl, or naphthyridinyl each optionally substituted with 1, 2, 3, 4, or 5 substituents selected independently from the group consisting of F, Cl, Br, —OCH₃, —OCH₂CH₃, —OCH(CH₃)₂, —NO₂, —CH₃, and —CF₃.

In some embodiments, Ar is selected from the group consisting of 3-chloro-thiophen-2-yl, benzothiophen-2-yl, 2,5-dichloro-thiophen-3-yl, and 2-trifluoromethyl-[1,6]naphthyridin-3-yl.

In some embodiments, compounds of the present invention are represented by Formula (IIa):

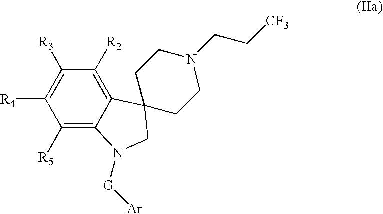

wherein each variable in Formula (IIa) are as defined herein infra and supra.

In some embodiments, compounds of the present invention are represented by Formula (IIa) or a pharmaceutically acceptable salt, solvate or hydrate thereof;

wherein:

G is C(═O) or S(═O)₂;

R₂, R₄, and R₅ are each independently H, —CH₃, or F;

R₃ is selected from the group consisting of H, —OCH₃, —CH₃, —CH₂CH₃, —CH(CH₃)₂, —C(CH₃)₃, —S(═O)₂NH₂, —S(═O)₂NHCH₃, —S(═O)CH₃, —S(═O)₂CH₃, —SCH₃, —C(═O)NH₂, —C≡N, —C(═O)N(CH₃)₂, —OCF₃, —CF₃, —CF₂CF₃, F, Cl, Br, I, —S(═O)CF₃, —S(═O)₂CF₃, —SCF₃, —OH, and —S(═O)₂NH₂; and

Ar is selected from the group consisting of 2-chlorophenyl, 4-methoxyphenyl, 4-nitrophenyl, 3,4-difluorophenyl, 2-fluorophenyl, 4-chlorophenyl, 3-dichloromethylphenyl, 3,5-bis-trifluoromethylphenyl, 2,4-difluorophenyl, 2-methoxyphenyl, 3,5-dimethoxyphenyl, 3,4,5-trimethoxyphenyl, 3-fluorophenyl, 4-fluorophenyl, 2,3-difluorophenyl, 2,5-difluorophenyl, 3-trifluoromethyl-4-fluorophenyl, 3-trifluoromethylphenyl, phenyl, 3-nitrophenyl, 3-nitro-4-methylphenyl, naphthalen-1-yl, naphthalen-2-yl, 4-trifluoromethylphenyl, 3-chlorophenyl, 3,5-dichlorophenyl, 2,4-dichlorophenyl, 2,6-difluorophenyl, 2,6-dichlorophenyl, 2,6-bis-trifluoromethylphenyl, 2-chloro-6-fluoro-phenyl, and 2,3-dichlorophenyl.

In some embodiments, compounds of the present invention are represented by Formula (IIIa):

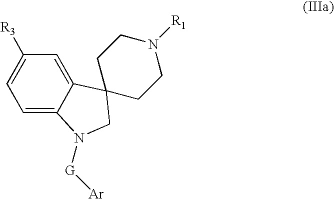

or a pharmaceutically acceptable salt, solvate or hydrate thereof;

wherein:

R₁ is n-propyl optionally substituted with fluorine;

G is C(═O) or S(═O)₂;

R₃ is —CH(CH₃)₂ or —C(CH₃)₃; and

Ar is selected from the group consisting of 2-chlorophenyl, 4-methoxyphenyl, 4-nitrophenyl, 3,4-difluorophenyl, 2-fluorophenyl, 4-chlorophenyl, 3-dichloromethylphenyl, 3,5-bis-trifluoromethylphenyl, 2,4-difluorophenyl, 2-methoxyphenyl, 3,5-dimethoxyphenyl, 3,4,5-trimethoxyphenyl, 3-fluorophenyl, 4-fluorophenyl, 2,3-difluorophenyl, 2,5-difluorophenyl, 3-trifluoromethyl-4-fluorophenyl, 3-trifluoromethylphenyl, phenyl, 3-nitrophenyl, 3-nitro-4-methylphenyl, naphthalen-1-yl, naphthalen-2-yl, 4-trifluoromethylphenyl, 3-chlorophenyl, 3,5-dichlorophenyl, 2,4-dichlorophenyl, 2,6-difluorophenyl, 2,6-dichlorophenyl, 2,6-bis-trifluoromethylphenyl, 2-chloro-6-fluoro-phenyl, and 2,3-dichlorophenyl.

In some embodiments, compounds of the present invention are represented by Formula (IVa):

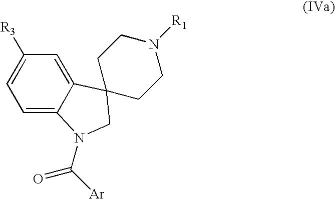

or a pharmaceutically acceptable salt, solvate or hydrate thereof;

R₁ is n-propyl optionally substituted with fluorine;

R₃ is —CH(CH₃)₂ or —C(CH₃)₃; and

Ar is selected from the group consisting of 2-chlorophenyl, 4-methoxyphenyl, 4-nitrophenyl, 3,4-difluorophenyl, 2-fluorophenyl, 4-chlorophenyl, 3-dichloromethylphenyl, 3,5-bis-trifluoromethylphenyl, 2,4-difluorophenyl, 2-methoxyphenyl, 3,5-dimethoxyphenyl, 3,4,5-trimethoxyphenyl, 3-fluorophenyl, 4-fluorophenyl, 2,3-difluorophenyl, 2,5-difluorophenyl, 3-trifluoromethyl-4-fluorophenyl, 3-trifluoromethylphenyl, phenyl, 3-nitrophenyl, 3-nitro-4-methylphenyl, naphthalen-1-yl, naphthalen-2-yl, 4-trifluoromethylphenyl, 3-chlorophenyl, 3,5-dichlorophenyl, 2,4-dichlorophenyl, 2,6-difluorophenyl, 2,6-dichlorophenyl, 2,6-bis-trifluoromethylphenyl, 2-chloro-6-fluoro-phenyl, and 2,3-dichlorophenyl.

In some embodiments, compounds of the present invention are represented by Formula (Va):

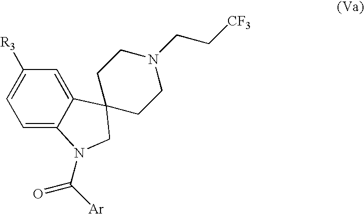

or a pharmaceutically acceptable salt, solvate or hydrate thereof;

wherein:

R₃ is —CH(CH₃)₂, or —C(CH₃)₃; and

Ar is phenyl or naphthyl each optionally substituted with 1, 2, 3, 4, 5, 6, or 7 substituents selected independently from the group consisting of F, Cl, Br, —OCH₃, —OCH₂CH₃, —OCH(CH₃)₂, —NO₂, —CH₃, —S(═O)₂CH₃, —S(═O)₂NH₂, —C≡N, —CF₃, and —OCF₃.

In some embodiments, compounds of the present invention are represented by Formula (Va):

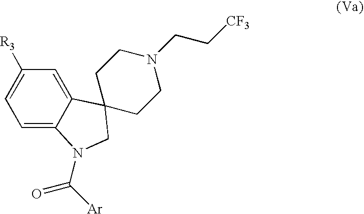

or a pharmaceutically acceptable salt, solvate or hydrate thereof;

wherein:

R₃ is —CH(CH₃)₂, —C(CH₃)₃, CF₃, or Cl; and

Ar is selected from the group consisting of 2-chlorophenyl, 2-fluorophenyl, 2,4-difluorophenyl, 2-methoxyphenyl, 2,3-difluorophenyl, 2,5-difluorophenyl, naphthalen-1-yl, 2,4-dichlorophenyl, 2,6-difluorophenyl, 2,6-dichlorophenyl, 2,6-bis-trifluoromethylphenyl, 2-chloro-6-fluoro-phenyl, and 2,3-dichlorophenyl.

In some embodiments, compounds of the present invention are represented by Formula (Va):

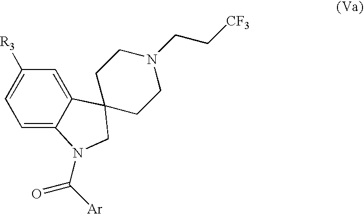

or a pharmaceutically acceptable salt, solvate or hydrate thereof;

wherein:

R₃ is —CH(CH₃)₂, —C(CH₃)₃, CF₃, or Cl; and

Ar is thienyl, pyridinyl, furanyl, pyrazolyl, benzofuranyl, benzothiophenyl, or naphthyridinyl each optionally substituted with 1, 2, 3, 4, or 5 substituents selected independently from the group consisting of F, Cl, Br, —OCH₃, —OCH₂CH₃, —OCH(CH₃)₂, —NO₂, —CH₃, and —CF₃.

### Illustrative Compounds of the Invention

Set forth below are illustrative compounds of the invention and their corresponding chemical names.

| Cmpd | | |
|---|---|---|
| No. | Chemical Structure | Chemical Name |
| 1 | 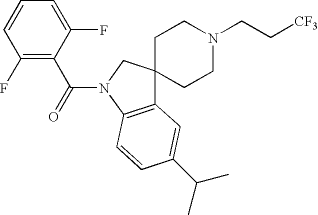 | 1′-(3,3,3-trifluoro-propyl)-1,2-dihydro-5-isopropyl-1-(2,6-difluoro-benzoyl)-spiro[3H-indole-3,4′-piperidine] |
| 2 | 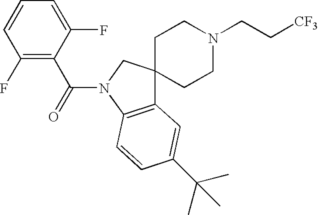 | 1′-(3,3,3-trifluoro-propyl)-1,2-dihydro-5-tert-butyl-1-(2,6-difluoro-benzoyl)-spiro[3H-indole-3,4′-piperidine] |
| 3 | 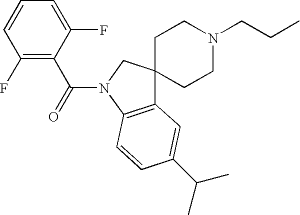 | 1′-(propyl)-1,2-dihydro-5-isopropyl-1-(2,6-difluoro-benzoyl)-spiro[3H-indole-3,4′-piperidine] |
| 4 | 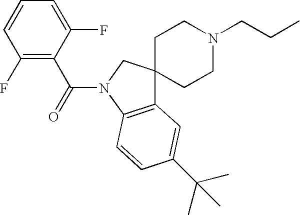 | 1′-(propyl)-1,2-dihydro-5-tert-butyl-1-(2,6-difluoro-benzoyl)-spiro[3H-indole-3,4′-piperidine] |
| 5 | 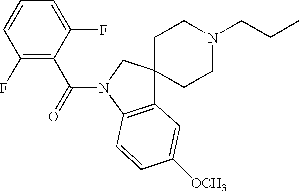 | 1′-(propyl)-1,2-dihydro-5-methoxy-1-(2,6-difluoro-benzoyl)-spiro[3H-indole-3,4′-piperidine] |
| 6 | 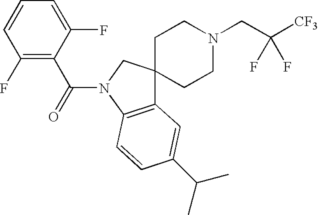 | 1′-(2,2,3,3,3-pentafluoro-propyl)-1,2-dihydro-5-isopropyl-1-(2,6-difluoro-benzoyl)-spiro[3H-indole-3,4′-piperidine] |
| 7 | 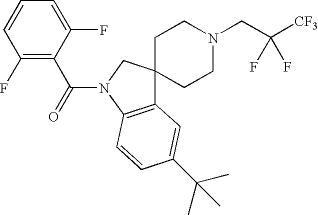 | 1′-(2,2,3,3,3-pentafluoro-propyl)-1,2-dihydro-5-tert-butyl-1-(2,6-difluoro-benzoyl)-spiro[3H-indole-3,4′-piperidine] |
| 8 | 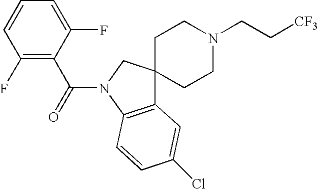 | 1′-(3,3,3-trifluoro-propyl)-1,2-dihydro-5-chloro-1-(2,6-difluoro-benzoyl)-spiro[3H-indole-3,4′-piperidine] |
| 9 | 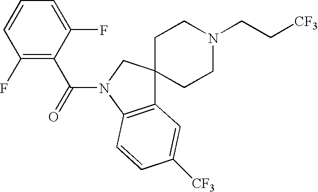 | 1′-(3,3,3-trifluoro-propyl)-1,2-dihydro-5-trifluoromethyl-1-(2,6-difluoro-benzoyl)-spiro[3H-indole-3,4′-piperidine] |
| 10 | 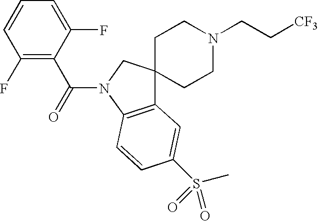 | 1′-(3,3,3-trifluoro-propyl)-1,2-dihydro-5-methanesulfonyl-1-(2,6-difluoro-benzoyl)-spiro[3H-indole-3,4′-piperidine] |
| 11 | 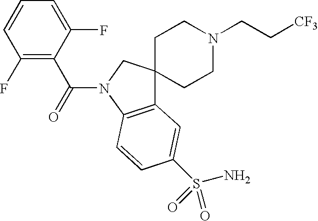 | 1′-(3,3,3-trifluoro-propyl)-1,2-dihydro-5-sulfamoyl-1-(2,6-difluoro-benzoyl)-spiro[3H-indole-3,4′-piperidine] |
| 12 | 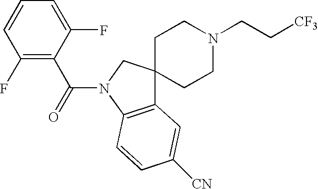 | 1′-(3,3,3-trifluoro-propyl)-1,2-dihydro-5-cyano-1-(2,6-difluoro-benzoyl)-spiro[3H-indole-3,4′-piperidine] |
| 13 | 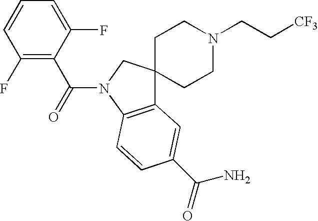 | 1′-(3,3,3-trifluoro-propyl)-1,2-dihydro-5-carbamoyl-1-(2,6-difluoro-benzoyl)-spiro[3H-indole-3,4′-piperidine] |
| 14 | 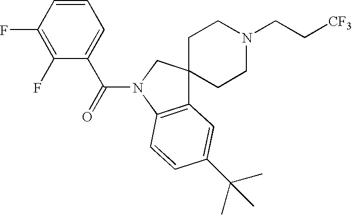 | 1′-(3,3,3-trifluoro-propyl)-1,2-dihydro-5-tert-butyl-1-(2,3-difluoro-benzoyl)-spiro[3H-indole-3,4′-piperidine] |
| 15 | 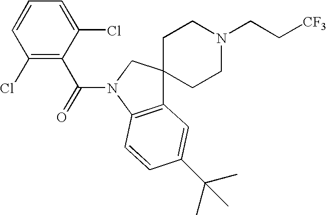 | 1′-(3,3,3-trifluoro-propyl)-1,2-dihydro-5-tert-butyl-1-(2,6-dichloro-benzoyl)-spiro[3H-indole-3,4′-piperidine] |
| 16 | 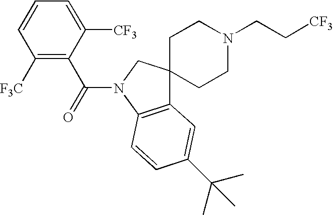 | 1′-(3,3,3-trifluoro-propyl)-1,2-dihydro-5-tert-butyl-1-(2,6-bis-trifluoromethyl-benzoyl)-spiro[3H-indole-3,4′-piperidine] |
| 17 | 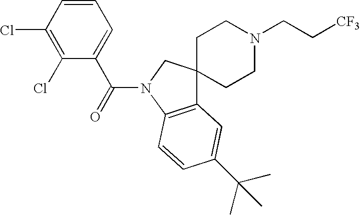 | 1′-(3,3,3-trifluoro-propyl)-1,2-dihydro-5-tert-butyl-1-(2,3-dichloro-benzoyl)-spiro[3H-indole-3,4′-piperidine] |
| 18 | 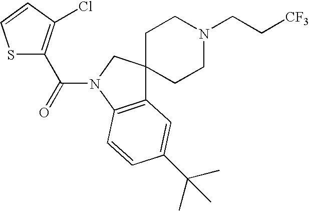 | 1′-(3,3,3-trifluoro-propyl)-1,2-dihydro-5-tert-butyl-1-(3-chloro-thiophene-2-carbonyl)-spiro[3H-indole-3,4′-piperidine] |
| 19 | 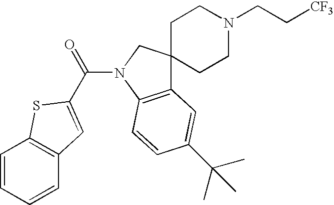 | 1′-(3,3,3-trifluoro-propyl)-1,2-dihydro-5-tert-butyl-1-(benzothiophene-2-carbonyl)-spiro[3H-indole-3,4′-piperidine] |
| 20 | 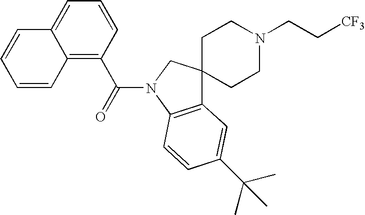 | 1′-(3,3,3-trifluoro-propyl)-1,2-dihydro-5-tert-butyl-1-(naphthalene-1-carbonyl)-spiro[3H-indole-3,4′-piperidine] |
| 21 | 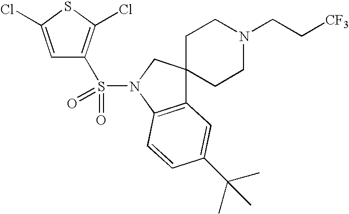 | 1′-(3,3,3-trifluoro-propyl)-1,2-dihydro-5-tert-butyl-1-(2,5-dichloro-thiophene-3-sulfonyl)-spiro[3H-indole-3,4′-piperidine] |
| 22 | 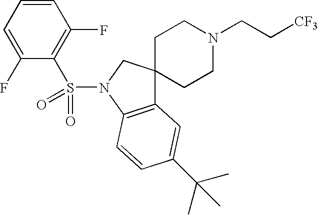 | 1′-(3,3,3-trifluoro-propyl)-1,2-dihydro-5-tert-butyl-1-(2,6-difluoro-benzenesulfonyl)-spiro[3H-indole-3,4′-piperidine] |
| 23 | 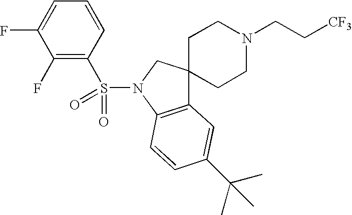 | 1′-(3,3,3-trifluoro-propyl)-1,2-dihydro-5-tert-butyl-1-(2,3-difluoro-benzenesulfonyl)-spiro[3H-indole-3,4′-piperidine] |
| 24 | 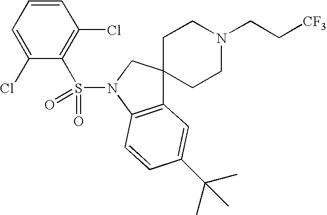 | 1′-(3,3,3-trifluoro-propyl)-1,2-dihydro-5-tert-butyl-1-(2,6-dichloro-benzenesulfonyl)-spiro[3H-indole-3,4′-piperidine] |
| 25 | 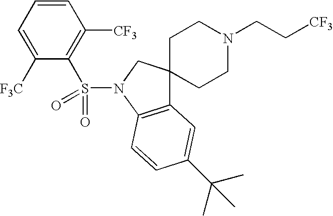 | 1′-(3,3,3-trifluoro-propyl)-1,2-dihydro-5-tert-butyl-1-(2,6-bis-trifluoromethyl-benzenesulfonyl)-spiro[3H-indole-3,4′-piperidine] |
| 26 | 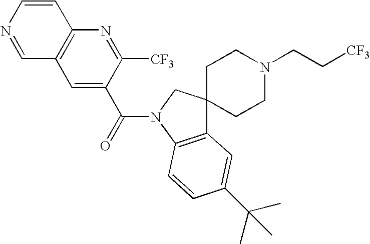 | 1′-(3,3,3-trifluoro-propyl)-1,2-dihydro-5-tert-butyl-1-(2-trifluoromethyl-[1,6]naphthyridine-3-carbonyl)-spiro[3H-indole-3,4′-piperidine] |
| 27 | 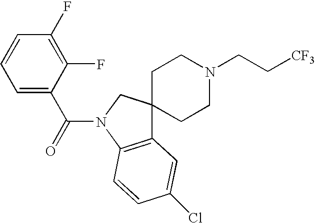 | 1′-(3,3,3-trifluoro-propyl)-1,2-dihydro-5-chloro-1-(2,3-difluoro-benzoyl)-spiro[3H-indole-3,4′-piperidine] |
| 28 | 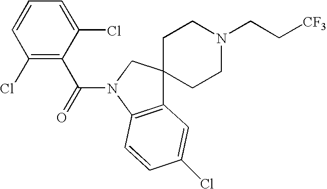 | 1′-(3,3,3-trifluoro-propyl)-1,2-dihydro-5-chloro-1-(2,6-dichloro-benzoyl)-spiro[3H-indole-3,4′-piperidine] |
| 29 | 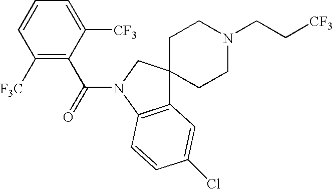 | 1′-(3,3,3-trifluoro-propyl)-1,2-dihydro-5-chloro-1-(2,6-bis-trifluoromethyl-benzoyl)-spiro[3H-indole-3,4′-piperidine] |
| 30 | 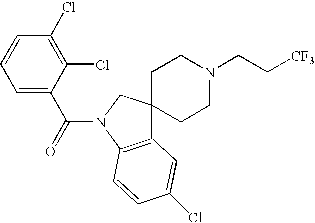 | 1′-(3,3,3-trifluoro-propyl)-1,2-dihydro-5-chloro-1-(2,3-dichloro-benzoyl)-spiro[3H-indole-3,4′-piperidine] |
| 31 | 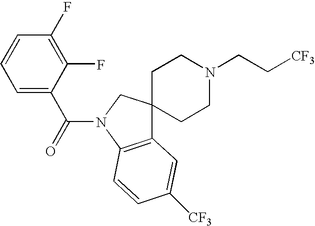 | 1′-(3,3,3-trifluoro-propyl)-1,2-dihydro-5-trifluoromethyl-1-(2,3-difluoro-benzoyl)-spiro[3H-indole-3,4′-piperidine] |
| 32 | 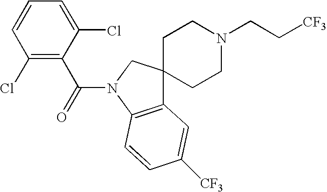 | 1′-(3,3,3-trifluoro-propyl)-1,2-dihydro-5-trifluoromethyl-1-(2,6-dichloro-benzoyl)-spiro[3H-indole-3,4′-piperidine] |
| 33 | 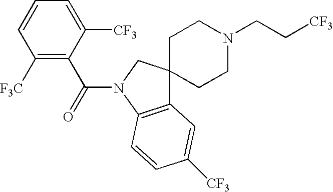 | 1′-(3,3,3-trifluoro-propyl)-1,2-dihydro-5-trifluoromethyl-1-(2,6-bis-trifluoromethyl-benzoyl)-spiro[3H-indole-3,4′-piperidine] |
| 34 | 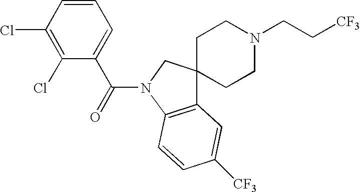 | 1′-(3,3,3-trifluoro-propyl)-1,2-dihydro-5-trifluoromethyl-1-(2,3-dichloro-benzoyl)-spiro[3H-indole-3,4′-piperidine] |

Additionally, compounds of the present invention, as described herein, encompass all pharmaceutically acceptable salts, solvates, and particularly hydrates, thereof.

### DEFINITIONS

As used herein, “antagonist” means a compound that competitively binds to the receptor at the same site as an agonist but which does not activate an intracellular response, and can thereby inhibit an intracellular response elicited by an agonist. An antagonist does not diminish the baseline intracellular response in the absence of an agonist. In some embodiments, an antagonist is a material not previously known to compete with an agonist to inhibit a cellular response when it binds to the receptor.

As used herein, “inverse agonist” means a compound that binds either to an endogenous form or to a constitutively activated form of a receptor so as to reduce the baseline intracellular response of the receptor observed in the absence of an agonist.

As used herein, “contacting” means bringing at least two moieties together, whether in an in vitro system or an in vivo system. As used herein, an in vitro system means outside of a living cell and in vivo means in a living cell or organism.

As used herein, “agonist” means a compound that activates an intracellular response when it binds to a receptor. A partial agonist is material (for example, a ligand or candidate compound) that activates an intracellular response when it binds to the receptor but to a lesser degree or extent than do full agonists.

The phrase “pharmaceutically acceptable salt,” as used herein, is a salt formed from an acid and a basic nitrogen group of one of the compounds of the invention. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. The term “pharmaceutically acceptable salt” also refers to a salt prepared from a Compound of the Invention having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia and organic amines, such as unsubstituted or hydroxy-substituted mono-, di- or trialkylamines; dicyclohexylamine; tributyl amine; pyridine; N-methyl-N-ethylamine; diethylamine; triethylamine; mono-, bis- or tris-(2-hydroxy-lower alkyl amines), such as mono-, bis- or tris-(2-hydroxyethyl)amine, 2-hydroxy-tert-butylamine or tris-(hydroxymethyl)methylamine, N,N-di-lower alkyl-N-(hydroxy lower alkyl)-amines, such as N,N-dimethyl-N-(2-hydroxyethyl)amine or tri-(2-hydroxyethyl)amine; N-methyl-D-glucamine; and amino acids such as arginine, lysine and the like.

As used herein and unless otherwise indicated, the term “hydrate” means a Compound of the Invention, or a salt thereof, that further includes a stoichiometric or non-stoichiometric amount of water bound by non-covalent intermolecular forces.

As used herein and unless otherwise indicated, the term “prodrug” means a Compound of the Invention derivative that can be hydrolyzed, oxidized, or otherwise reacted under biological conditions (in vitro or in vivo) to provide an active compound, particularly a Compound of the Invention. Examples of prodrugs include, but are not limited to, derivatives and metabolites of a Compound of the Invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Preferably, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

The terms “isotopically” or “radio-labeled” refer to Compounds of the Invention which are identical to the Compounds of the Invention disclosed herein, but for the fact that one or more atoms are replaced or substituted by an atom having an atomic mass or mass number different from the atomic mass or mass number typically found in nature (i.e., naturally occurring) including, but not limited to, ²H (also written as D for deuterium), 3H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I.

An “individual” or “patient” is defined herein to include any animal, in one embodiment is a vertebrate, in another embodiment is a mammal (non-primate and primate), and examples include but not limited to cow, horse, sheep, pig, chicken, turkey, quail, cat, dog, mouse, rat, rabbit, guinea pig, monkey, and the like. In another embodiment, is a human and in certain embodiments, the human is an infant, child, adolescent or adult. In a particular embodiment, the patient is at risk for a vascular, cardiovascular or neurological disease or disorder. Patients who are at risk include, but are not limited to, those with hereditary history of a vascular, cardiovascular or neurological disease or disorder, or in a state of physical health which puts them at risk for a vascular, cardiovascular or neurological disease or disorder. In another embodiment, the patient has previously had a stroke or is at risk to have a stroke.

The phrase “effective amount” when used in connection with a Compound of the Invention means an amount effective for: (a) treating, preventing or managing a vascular or cardiovascular disease or disorder or a neurological disease or disorder; (b) preventing or reducing damage caused by a vascular or cardiovascular disease or disorder or a neurological disease or disorder; (c) inhibiting Mas receptor function in a cell capable of expressing Mas; or (d) detection by an instrument useful for detecting and/or measuring radioactivity (e.g., a liquid scintillation counter).

The phrases “treatment of” “treating” and the like include the amelioration or cessation of a vascular or cardiovascular disease or disorder or a neurological disease or disorder. In one embodiment, treating includes inhibiting, for example, decreasing the overall frequency of episodes of a cardiovascular disease or disorder or a neurological disease or disorder.

The phrases “prevention of” “preventing” and the like include the avoidance of the onset of a vascular or cardiovascular disease or disorder or a neurological disease or disorder. In one embodiment, neurological or vascular damage caused by stroke is prevented.

The phrases “management of”, “managing” and the like include the prevention of worsening of a vascular or cardiovascular disease or disorder or a neurological disease or disorder, or a symptom thereof.

### Chemical Group, Moiety or Radical:

The term “C₁₋₆ acyl” refers to a C₁₋₆ alkyl radical attached directly to the carbon of a carbonyl group wherein the definition for alkyl is as described herein; some examples include, but not limited to, acetyl, propionyl, n-butanoyl, iso-butanoyl, sec-butanoyl, t-butanoyl (also referred to as pivaloyl), pentanoyl and the like.

The term “C₁₋₆ acyloxy” refers to an acyl radical attached directly to an oxygen atom [forming: —OC(═O)—C₁₋₆ alkyl] wherein acyl has the same definition has described herein; some examples include but not limited to acetyloxy [—OC(═O)CH₃], propionyloxy, butanoyloxy, iso-butanoyloxy, sec-butanoyloxy, t-butanoyloxy and the like.

The term “C₂₋₆ alkenyl” refers to a radical containing 2 to 6 carbons wherein at least one carbon-carbon double bond is present, some embodiments are 2 to 4 carbons, some embodiments are 2 to 3 carbons, and some embodiments have 2 carbons. Both E and Z isomers are embraced by the term “alkenyl.” Furthermore, the term “alkenyl” includes di- and tri-alkenyls. Accordingly, if more than one double bond is present then the bonds may be all E or Z or a mixtures of E and Z. Examples of an alkenyl include vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2,4-hexadienyl and the like.

The term “C₁₋₆ alkoxy” refers to an alkyl radical, as defined herein, attached directly to an oxygen atom (i.e., —O—C₁₋₆ alkyl). Examples include methoxy, ethoxy, n-propoxy, iso-propoxy, n-butoxy, t-butoxy, iso-butoxy, sec-butoxy and the like.

The term “C₁₋₆ alkoxycarbonyl” refers to an alkoxy group attached directly to the carbon of a carbonyl and can be represented by the formula —C(═O)O—C₁₋₆-alkyl, wherein the C₁₋₆ alkyl group is as defined herein. Examples include, but not limited to, methoxycarbonyl, ethoxycarbonyl, propoxycarbonyl, iso-propoxycarbonyl, butoxycarbonyl, sec-butoxycarbonyl, iso-butoxycarbonyl, t-butoxycarbonyl, n-pentoxycarbonyl, iso-pentoxycarbonyl, t-pentoxycarbonyl, neo-pentoxycarbonyl, n-hexyloxycarbonyl, and the like.

The term “C₁₋₆ alkyl” refers to a straight or branched carbon radical containing 1 to 6 carbons, some embodiments are 1 to 5 carbons, some embodiments are 1 to 4 carbons, some embodiments are 1 to 3 carbons, and some embodiments are 1 or 2 carbons. Examples of an alkyl include, but not limited to, methyl, ethyl, n-propyl, iso-propyl, n-butyl, iso-butyl, t-butyl, sec-butyl, n-pentyl, iso-pentyl, sec-pentyl, neo-pentyl, pent-3-yl, 2-methyl-but-1-yl, 1,2-dimethyl-prop-1-yl, n-hexyl, iso-hexyl, sec-hexyl, neo-hexyl, 1-ethyl-2-methyl-prop-1-yl, 1,2,2-trimethyl-prop-1-yl, 1,1,2-trimethyl-prop-1-yl, 1-ethyl-1-methyl-prop-1-yl, 1,1-dimethyl-but-1-yl, 1,2-dimethyl-but-1-yl, 2,3-dimethyl-but-1-yl, 2,2-dimethyl-but-1-yl, 1,3-dimethyl-but-1-yl, hex-3-yl, 2-methyl-pent-1-yl, 3-methyl-pent-1-yl, and the like.

The term “C₁₋₆ alkylamino” refers to one alkyl radical attached directly to an amino radical (i.e., —HN—C₁₋₆ alkyl) wherein the alkyl radical has the same meaning as described herein. Some examples include, but not limited to, methylamino (i.e., —HNCH₃), ethylamino, n-propylamino, iso-propylamino, n-butylamino, sec-butylamino, iso-butylamino, t-butylamino, and the like.

The term “C₁₋₆ alkylcarboxamide” or “C₁₋₆ alkylcarboxamido” refers to a single C₁₋₆ alkyl group attached to the nitrogen of an amide group, wherein alkyl has the same definition as described herein. The C₁₋₆ alkylcarboxamido may be represented by the following:

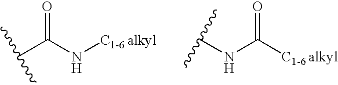

Examples include, but not limited to, N-methylcarboxamide, N-ethylcarboxamide, N-n-propylcarboxamide, N-iso-propylcarboxamide, N-n-butylcarboxamide, N-sec-butylcarboxamide, N-iso-butylcarboxamide, N-t-butylcarboxamide and the like.

The term “C₁₋₆ alkylsulfinyl” refers to an alkyl radical attached directly to a sulfoxide radical of the formula: —S(═O)— wherein the alkyl radical has the same definition as described herein. Examples include, but not limited to, methylsulfinyl, ethylsulfinyl, n-propylsulfinyl, iso-propylsulfinyl, n-butylsulfinyl, sec-butylsulfinyl, iso-butylsulfinyl, t-butyl, and the like.

The term “C₁₋₆ alkylsulfonamide” refers to the groups

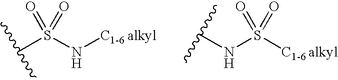

wherein C₁₋₆ alkyl has the same definition as described herein.

The term “C₁₋₆ alkylsulfonyl” refers to an alkyl radical attached to a sulfone radical of the formula: —S(O)₂— wherein the alkyl radical has the same definition as described herein. Examples include, but not limited to, methylsulfonyl, ethylsulfonyl, n-propylsulfonyl, iso-propylsulfonyl, n-butylsulfonyl, sec-butylsulfonyl, iso-butylsulfonyl, t-butyl, and the like.

The term “C₁₋₆ alkylthio” refers to an alkyl radical attached to a sulfide of the formula: —S— wherein the alkyl radical has the same definition as described herein. Examples include, but not limited to, methylsulfanyl (i.e., CH₃S—), ethylsulfanyl, n-propylsulfanyl, iso-propylsulfanyl, n-butylsulfanyl, sec-butylsulfanyl, iso-butylsulfanyl, t-butyl, and the like.

The term “C₁₋₆ alkylthiocarboxamide” refers to an alkyl attached directly to a thiocarboxamide group at either the nitrogen or at the carbon of the thiocarbonyl and has the following respective formulae:

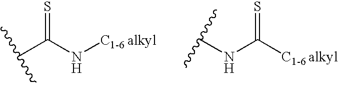

wherein C₁₋₆ alkyl has the same definition as described herein.

The term “C₁₋₆ alkylthioureyl” refers to the group of the formula: —NC(═S)NH— wherein one are both of the nitrogens are independently substituted with the same or different C₁₋₆ alkyl groups and alkyl has the same definition as described herein. Examples of an alkylthioureyl include, but not limited to, CH₃NHC(S)NH—, NH₂C(S)NCH₃—, (CH₃)₂N(S)NH—, (CH₃)₂N(S)NH—, (CH₃)₂N(S)NCH₃—, CH₃CH₂NHC(S)NH—, CH₃CH₂NHC(S)NCH₃—, and the like.

The term “C₁₋₆ alkylureyl” refers to the group of the formula: —NC(═O)NH— wherein one are both of the nitrogens are independently substituted with the same or different C₁₋₆ alkyl group wherein alkyl has the same definition as described herein. Examples of an alkylureyl include, but not limited to, CH₃NHC(O)NH—, NH₂C(O)NCH₃—, (CH₃)₂N(O)NH—, (CH₃)₂N(O)NH—, (CH₃)₂N(O)NCH₃—, CH₃CH₂NHC(O)NH—, CH₃CH₂NHC(O)NCH₃—, and the like.

The term “C₂₋₆ alkynyl” refers to a radical containing 2 to 6 carbons and at least one carbon-carbon triple bond (—C≡C—), some embodiments are 2 to 4 carbons, some embodiments are 2 to 3 carbons, and some embodiments have 2 carbons (—C≡CH). Examples of a C₂₋₆ alkynyl include, but not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term C₂₋₆ alkynyl includes di- and tri-ynes.

The term “amino” refers to the group —NH₂.

The term “aryl” refers to an aromatic ring radical containing 6 to 10 ring carbons. Examples include phenyl, naphthyl, and the like.

The term “carboxamide” refers to the group —C(═O)NH₂.

The term “carboxy” or “carboxyl” refers to the group —CO₂H; also referred to as a carboxylic acid group.

The term “cyano” refers to the group —CN.

The term “C₃₋₆ cycloalkyl” refers to a saturated carbocyclic ring radical containing 3 to 6 carbons; some embodiments contain 3 to 5 carbons (“C₃₋₅ cycloalkyl”); some embodiments contain 3 to 4 carbons (“C₃₋₄ cycloalkyl”). Examples include cyclopropyl, cyclobutyl, cyclopentyl, cyclopenyl, cyclohexyl, and the like.

The term “di-C₁₋₆-dialkylamino” refers to an amino substituted with two of the same or different C₁₋₆ alkyl radicals wherein alkyl radical has the same definition as described herein. Some examples include, but not limited to, dimethylamino, methylethylamino, diethylamino, methylpropylamino, methylisopropylamino, ethylpropylamino, ethylisopropylamino, dipropylamino, propylisopropylamino and the like.

The term “di-C₁₋₆-alkylcarboxamide” or “di-C₁₋₆-alkylcarboxamido” refers to two C₁₋₆ alkyl radicals, that are the same or different, attached to an amide group, wherein alkyl has the same definition as described herein. A di-C₁₋₆-alkylcarboxamido can be represented by the following groups:

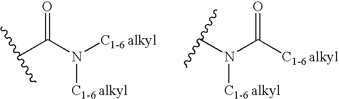

wherein C₁₋₆ alkyl has the same definition as described herein. Examples of a dialkylcarboxamide include, but not limited to, N,N-dimethylcarboxamide, N-methyl-N-ethylcarboxamide, N,N-diethylcarboxamide, N-methyl-N-isopropylcarboxamide, and the like.

The term “di-C₁₋₆-alkylsulfonamide” refers to two C₁₋₆ alkyl radicals, that are the same or different, attached to a sulfonamide group, wherein alkyl has the same definition as described herein. A di-C₁₋₆-alkylsulfonamide can be represented by the following groups:

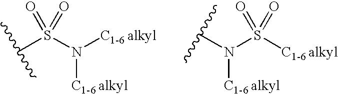

Examples include, but not limited to, dimethylsulfamoyl [—S(═O)₂N(CH₃)₂], ethylmethylsulfamoyl, methanesulfonyl-methyl-amino [—N(CH₃)S(═O)₂CH₃], ethyl-methanesulfonyl-amino [—N(CH₂CH₃)S(═O)₂CH₃], and the like.

The term “di-C₁₋₆-alkylthiocarboxamido” or “di-C₁₋₆-alkylthiocarboxamide” refers to two C₁₋₆ alkyl radicals, that are the same or different, attached to a thioamide group, wherein alkyl has the same definition as described herein. A C₁₋₆ dialkylthiocarboxamido can be represented by the following groups:

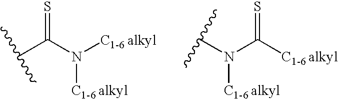

Examples of a dialkylthiocarboxamide include, but not limited to, N,N-dimethylthiocarboxamide, N-methyl-N-ethylthiocarboxamide and the like.

The term “C₁₋₆ haloalkoxy” refers to a haloalkyl, as defined herein, which is directly attached to an oxygen atom. Examples include, but not limited to, difluoromethoxy, trifluoromethoxy (OCF₃), 2,2,2-trifluoroethoxy, pentafluoroethoxy and the like.

The term “C₁₋₆ haloalkyl” refers to an alkyl group, defined herein, wherein the alkyl is substituted with one halogen up to fully substituted, a fully substituted haloalkyl can be represented by the formula CₙL₂₊₁ wherein L is a halogen and “n” is 1, 2, 3, 4, 5, or δ6 and when more than one halogen is present then they may be the same or different and selected from the group consisting of F, Cl, Br and I, in some embodiments, halogen is F. Examples of haloalkyl groups include, but not limited to, fluoromethyl, difluoromethyl, trifluoromethyl, chlorodifluoromethyl, 2,2,2-trifluoroethyl, pentafluoroethyl, and the like.

The term “C₁₋₆ haloalkylsulfinyl” refers to a C₁₋₆ haloalkyl radical directly attached to the sulfur of a sulfoxide group of the formula: —S(—O)—, wherein the C₁₋₆ haloalkyl radical has the same definition as described herein. Examples include, but not limited to, trifluoromethylsulfinyl, 2,2,2-trifluoroethylsulfinyl, 2,2-difluoroethylsulfinyl and the like.

The term “C₁₋₆ haloalkylsulfonyl” refers to a haloalkyl radical directly attached to the sulfur of a sulfone group of the formula: —S(═O)₂— wherein haloalkyl has the same definition as described herein. Examples include, but not limited to, trifluoromethylsulfonyl, 2,2,2-trifluoroethylsulfonyl, 2,2-difluoroethylsulfonyl and the like.

The term “C₁₋₆ haloalkylthio” refers to a haloalkyl radical directly attached to a sulfur wherein the haloalkyl has the same meaning as described herein. Examples include, but not limited to, trifluoromethylthio (i.e., CF₃S—), 1,1-difluoroethylthio, 2,2,2-trifluoroethylthio and the like.

The term “halogen” or “halo” refers to a fluoro, chloro, bromo or iodo group.

The term “heteroaryl” refers to an aromatic ring system that may be a single ring, two fused rings or three fused rings wherein at least one ring carbon is replaced with a heteroatom selected from, but not limited to, the group consisting of O, S, N, and NH. Examples of heteroaryl groups include, but not limited to, pyridyl, benzofuranyl, pyrazinyl, pyridazinyl, pyrimidinyl, triazinyl, quinoline, benzoxazole, benzothiazole, 1H-benzimidazole, isoquinoline, quinazoline, quinoxaline, pyrrole, indolyl, napthyridinyl, and the like. Other examples include, but not limited to, heteroaryl groups in TABLE 3, TABLE 4, and the like.

The term “heterocyclic” refers to a non-aromatic carbon ring such as, cycloalkyl or cycloalkenyl (a cycloalkyl with at least one endo-cyclic double bond), wherein one, two or three ring carbons are replaced by a heteroatom selected from, but not limited to, the group consisting of —O—, —S—, —S(═O)—, —S(═O)₂—, and —NH—, and the ring carbon atoms are optionally substituted with oxo or thiooxo thus forming a carbonyl or thiocarbonyl group respectively. The heterocyclic group can be a 3, 4, 5, or 6-member containing ring. Examples of a heterocyclic group include but not limited to aziridin-1-yl, aziridin-2-yl, azetidin-1-yl, azetidin-2-yl, azetidin-3-yl, piperidin-1-yl, piperidin-4-yl, morpholin-4-yl, piperzin-1-yl, piperzin-4-yl, pyrrolidin-1-yl, pyrrolidin-3-yl, [1,3]-dioxolan-2-yl and the like.

The term “hydroxyl” refers to the group —OH.

The term “nitro” refers to the group —NO₂.

The term “oxo” refers generally to a double bonded oxygen; typically “oxo” is a substitution on a carbon and together form a carbonyl group.

The term “phenyl” refers to the group C₆H₅—.

The term “sulfonamide” refers to the group —S(═O)₂NH₂.

The term “thiol” refers to the group —SH.

### Preparation of Compounds of the Invention

### General Synthetic Methods

The novel substituted 1,2-dihydro-spiro[3H-indole-3,4′-piperidine] compounds of the present invention can be readily prepared according to a variety of synthetic manipulations, all of which would be familiar to one skilled in the art. Certain methods for the preparation of compounds of the present invention include, but are not limited to, those described in Scheme 1-13.

One common intermediate, Indole A, can be used in the synthesis of novel substituted 1,2-dihydro-spiro[3H-indole-3,4′-piperidine] compounds of the present invention. Indole A can be prepared as shown in Schemes 1 and 2, infra. As illustrated in Scheme 1, the nitrogen of Alcohol B can be protected with a suitable protecting group (i.e., —PG₁) and the alcohol subsequently converted to Aldehyde C via an oxidation step. Suitable protecting groups for the nitrogen of Alcohol B include, but are not limited to, t-butyl carbamate (Boc), benzyl carbamate (Cbz), p-methoxybenzyl carbamate (Moz), allyl carbamate (Alloc), 9-fluorenylmethyl carbamate (Fmoc), and the like. Various methods can be used to protect the nitrogen of Alcohol B. For example, the t-butyl carbamate group can be introduced using a variety of reagents, such as (Boc)₂O, with a suitable base (such as, NaOH, KOH, or Me₄NOH) in a suitable solvent(s) (THF, CH₃CN, DMF, EtOH, MeOH, H₂O, or mixtures thereof) and at a temperature of about −10° C. to about 50° C. It is understood that a protecting group can also be a soluble or insoluble resin commonly used in the art in the preparation of compound libraries. Other representative protecting groups suitable for a wide variety of synthetic transformations are disclosed in Greene and Wuts, Protective Groups in Organic Synthesis, third edition, John Wiley & Sons, New York, 1999, the disclosure of which is incorporated herein by reference in its entirety. Suitable oxidizing methods for the conversion to Aldehyde C are known in the art and include for example, Swern or Swern-like oxidations, Corey oxidation with NCS or any other suitable procedures such as those described by Hudlicky, M. in Oxidations in Organic Chemistry, ACS Monograph 186 (1990), incorporated herein by reference in its entirety. One particularly useful oxidation procedure employs pyridine.SO3 and a tertiary amine (such as, N,N-diisopropylethylamine, triethylamine, or N-methylmorpholine) in a suitable solvent(s) (such as, THF, CH₂Cl₂, CH₃CN, DMF, or mixtures thereof). Suitable reaction temperatures range from about −20° C. to about 50° C., and about −5° C. to about 35° C.

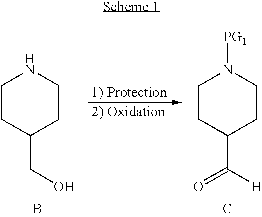

The method for the conversion of Aldehyde C to Indole A is shown in Scheme 2. A mixture of an appropriately substituted Arylhydrazine D and the N-protected Aldehyde C in the presence of an acid produces the intermediate indole (not shown). Suitable acids include, but are not limited to, trifluoroacetic acid, p-toluenesulfonic acid, and the like. Reduction of the resulting intermediate to the Indole A can be accomplished by a number of reducing agents. Suitable reducing agents include, but not limited to, alkali metal aluminum hydrides (such as, lithium aluminum hydride), alkali metal borohydrides (such as, sodium borohydride, lithium borohydride), alkali metal trialkoxyaluminum hydrides (such as, lithium tri-tert-butoxyaluminum hydride), and the like; see, e.g., Maligres, P. E.; Houpis, I.; Rossen, K.; Molina, A.; Sager, J.; Upadhyay, V.; Wells, K. M.; Reamer, R. A.; Lynch, J. E.; Askin, D.; Volante, R. P.; Reider, P. J. Tetrahedron 53:10983-10992 (1997)). The solvent includes ethereal solvents (such as, tetrahydrofuran or dioxane), aromatic solvents (such as, toluene), alcoholic solvents for use with primarily borohydride (such as, ethanol, methanol or isopropanol) or mixtures thereof. Suitable reaction temperatures range from about −78° C. to 120° C., and about −20° C. to 8° C.

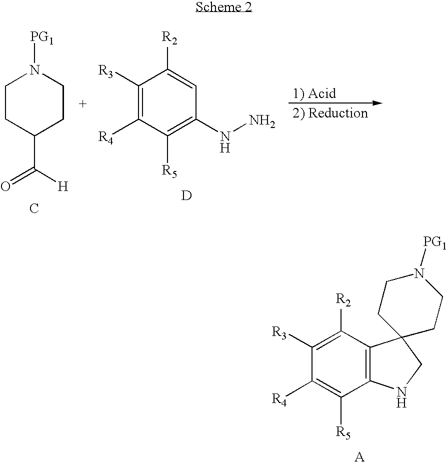

The common intermediate Indole A can also be prepared using the following the methods illustrated in Schemes 3-5, infra.

As shown in Scheme 3, Carboxylic acid E is converted to an acid halide [using (ClCO)₂, SOCl₂, SOBr₂, and the like, optionally in the presence of DMF] and subsequently coupled with Amine F using a base in an inert solvent to give amide G, wherein PG₁ is similar as described herein. Suitable coupling bases include alkali metal carbonate (such as, sodium carbonate or potassium carbonate, etc.), alkali metal hydrogencarbonate (such as, sodium hydrogencarbonate, potassium hydrogencarbonate, etc.), alkali hydroxide (such as, sodium hydroxide, potassium hydroxide, etc.), a tertiary amine (such as, N,N-diisopropylethylamine, triethylamine, N-methylmorpholine, etc.), or an aromatic amine (such as, pyridine, imidazole, poly-(4-vinylpyridine), etc.). The inert solvent includes lower halocarbon solvents (such as dichloromethane, dichloroethane, or chloroform, etc.), ethereal solvents (such as tetrahydrofuran or dioxane), amide solvents (such as N,N-dimethylformamide, etc.), or aromatic solvents (such as, toluene or pyridine, etc.) and mixtures thereof. Suitable reaction temperatures range from about −20° C. to 50° C., and about 0° C. to 40° C.

Alternatively, Carboxylic acid E is reacted with Amine F in the presence of a dehydrating condensing agent (i.e., coupling reagent) in an inert solvent with or without a base to provide the Amide G. Suitable coupling agents include dicyclohexylcarbodiimide (DCC), 1,3-diisopropylcarbodiimide (DIC), 1-ethyl-3-(3-dimethylaminopropyl)carbodiimide hydrochloride (EDC.HCl), bromo-tris-pyrrolidino-phosphonium hexafluorophosphate (PyBroP), O-(7-azabenzotriazol-1-yl)-1,1,3,3-tetramethyluronium hexafluorophosphate (HATU), benzotriazoloyloxytris(dimethylamino) phosphonium hexafluorophosphate (BOP), or 1-cyclohexyl-3-methylpolystyrene-carbodiimide. The base includes a tertiary amine (such as, N,N-diisopropylethylamine or triethylamine, etc.). The inert solvent includes lower halocarbon solvents (such as, dichloromethane, dichloroethane, chloroform, etc.), ethereal solvents (such as, tetrahydrofuran, dioxane), nitrile solvents (such as, acetonitrile, etc.), or amide solvents (such as, N,N-dimethylformamide, etc.). Optionally, 1-hydroxybenzotriazole (HOBT), HOBT-6-carboxamidomethyl polystyrene, or 1-hydroxy-7-azabenzotriazole (HOAT) can be used as a reactant agent. Suitable temperatures range from about −20° C. to 50° C., and about 0° C. to 40° C.

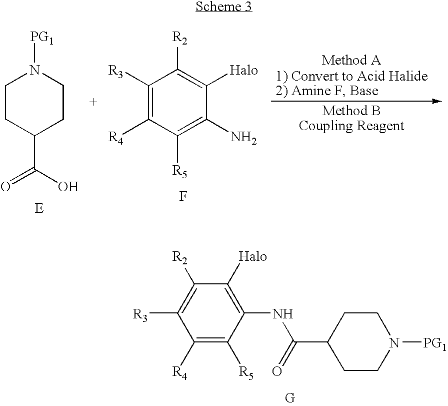

The nitrogen of Amide G is subsequently masked with an appropriate protecting group (PG₂) that is optionally orthogonal to the other protective group(s) in the molecule to give Protected Amide H, as illustrated in Scheme 4. Suitable groups for —PG₂ include, benzyl, p-methoxybenzyl, and the like. Other suitable protecting groups are described by Greene and Wuts, in Protective Groups in Organic Synthesis, third edition, John Wiley & Sons, New York, 1999, supra. It is understood that —PG₂ can also be a soluble or insoluble resin commonly used in the preparation of the compound libraries.

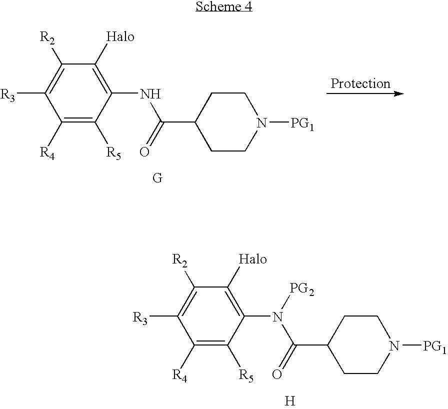

As shown in Scheme 5, the Protected Amide H can be cyclized by treatment with a strong base in the presence of a metal catalyst with a suitable ligand in a suitable solvent to give Lactam J as the intermediate. A similar process has been described by Lee, S.; and Hartwig, J. F. in J. Org. Chem. 2001, 66, 3402-3415, encorporated by reference in it's entirity. A suitable strong base is one that is appropriate to remove the α-hydrogen of the protected amide group, explicitly shown in Scheme 5. Suitable strong bases include, alkali metal alkoxide (such as, potassium tert-butoxide, sodium tert-butoxide, and the like); alkyl lithiums (such as, tert-butyl lithium, and the like). Suitable metal catalysts include palladium acetate, Pd(dbd)₃, and the like, and a suitable ligand includes tricyclohexyl phosphine (Cy₃P), BINAP, t-Bu₃P, carbene ligands known in the art, and the like. The inert solvent includes ethereal solvents (such as, tetrahydrofuran, dioxane and the like), aromatic solvents (such as, benzene, toluene, and the like), and mixtures thereof. Reaction temperatures range from about RT to about 200° C. The carbonyl of the resulting lactam is subsequently reduced with a reducing agent in an inert solvent to give Spirocyclic Diamine K. The reducing agent includes alkali metal aluminum hydrides (such as, lithium aluminum hydride), alkali metal borohydrides (such as, lithium borohydride), alkali metal trialkoxyaluminum hydrides (such as, lithium tri-tert-butoxyaluminum hydride), dialkylaluminum hydrides (such as, di-isobutylaluminum hydride), borane, dialkylboranes (such as, di-isoamyl borane), alkali metal trialkylborohydrides (such as, lithium triethylborohydride). The inert solvent includes ethereal solvents (such as, tetrahydrofuran or dioxane) or aromatic solvents (such as, toluene, etc.). Suitable reaction temperatures range from about −78° C. to 200° C., and about 50° C. to 120° C.

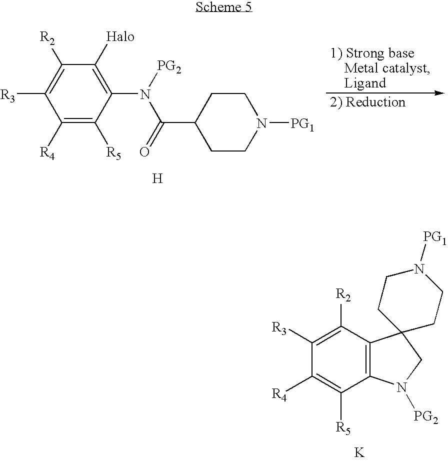

It is understood that Spirobicyclic Diamine K is a diprotected scaffold which is useful in the preparation of compounds of the invention. Particularly useful is when the two protecting groups are orthogonal to each other (i.e., each can substantially be removed selectively in the presence of the other). In general, this particular protecting strategy is illustrated in Scheme 6 to give Indoles A and L respectively. For convenience, the two protecting groups for Spirobicyclic Diamine K are selected so one protecting group can be substantially removed without substantially affecting the other protecting group. This type of strategy is referred to as orthogonal protection. One example includes when —PG₁ is a Boc group and —PG₂ is a benzyl group. In this example, the Boc group can be removed under acidic conditions without substantially affecting the benzyl group. Alternatively, the benzyl group can be removed under conditions that will not substantially remove the Boc group. Many orthogonal protection schemes are known in the art.

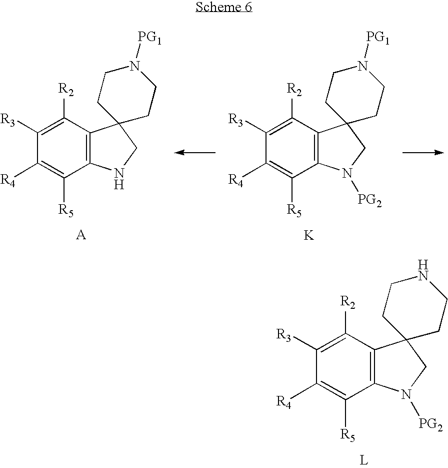

Alternatively, the common intermediate Indole A can also be prepared using the following the methods illustrated in Scheme 7, infra.

First, Amine M is substituted via reductive amination reaction using an aldehyde (ArCHO, wherein Ar is substituted or unsubstituted as described herein) and a reducing agent in an inert solvent with or without an acid. The reducing agent includes sodium triacetoxyborohydride, sodium cyanoborohydride, sodium borohydride, or borane-pyridine complex, such as, sodium triacetoxyborohydride or sodium cyanoborohydride. The inert solvent includes lower alkyl alcohol solvents (such as, methanol or ethanol, etc.), lower halocarbon solvents (such as, dichloromethane, dichloroethane, or chloroform, etc.), ethereal solvents (such as, tetrahydrofuran or dioxane), or aromatic solvents (such as, toluene, etc.). Optional acids include inorganic acid (such as, hydrochloric acid or sulfuric acid) or organic acid (such as, acetic acid). Suitable reaction temperatures range from about −20° C. to 120° C., such as, about 0° C. to 100° C. Also this reaction can be carried out under microwave conditions. The 2° amine (not shown) is subsequently coupled with a 2-haloacetic acid or 2-haloacetyl halide via a coupling reaction, such as reacting with chloroacetyl chloride, bromoacetyl bromide, chloroacetic acid anhydride, and the like. The resulting Amide N is cyclized via reaction with a suitable palladium catalyst to give the Lactam O (See, e.g., Hennessey, E. J.; Buchwald, S. L. J. Am. Chem. Soc. 125: 12084-12085 (2003)). Double alkylation with a protected bis-haloalkyl amine produces the Spiro Lactam P which can be reduced as described herein to give Indole A.

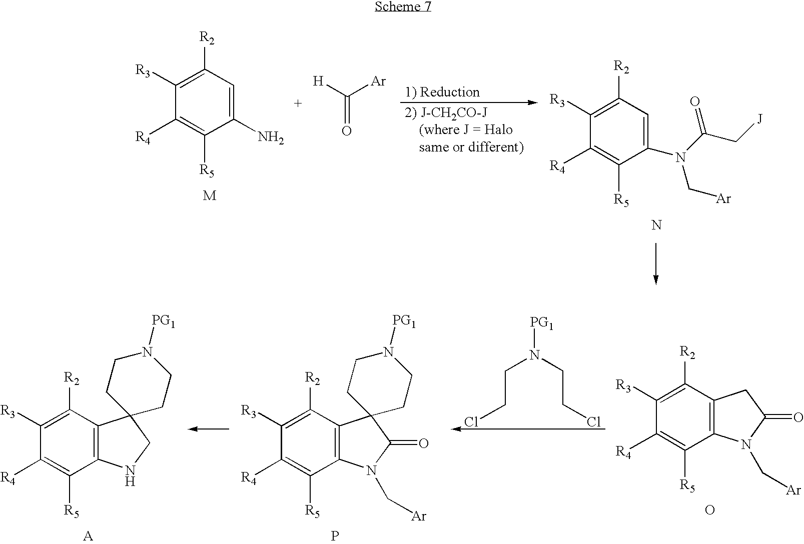

Outlined in Schemes 8-13 are methods for the preparation of intermediates useful in preparing novel substituted 1,2-dihydro-spiro[3H-indole-3,4′-piperidine] compounds.

As shown in Scheme 8, the common intermediate Indole A can be functionalized while PG₁ is still present. The common intermediate Indole A is reacted with a carboxylic acid (ArCO₂H, Ar has the same meaning as described herein) with a dehydrating condensing agent in an inert solvent with or without a base to provide Amide Q. The dehydrating condensing agent includes dicyclohexylcarbodiimide (DCC), 1,3-diisopropylcarbodiimide (DIC), 1-ethyl-3-(3-dimethylaminopropyl)carbodiimide hydrochloride (EDC.HCl), bromo-tris-pyrrolidino-phosphonium hexafluorophosphate (PyBroP), benzotriazoloyloxytris(dimethylamino)-phosphonium hexafluorophosphate (BOP), O-(7-azabenzo triazol-1-yl)-1,1,3,3-tetramethyluronium hexafluorophosphate (HATU), or 1-cyclohexyl-3-methylpolystyrene-carbodiimide. The base includes a tertiary amine (such as, N,N-diisopropylethylamine, triethylamine, and the like). The inert solvent includes lower halocarbon solvents (such as, dichloromethane, dichloroethane, chloroform, and the like), ethereal solvents (such as, tetrahydrofuran, dioxane, and the like), nitrile solvents (such as, acetonitrile, and the like), amide solvents (N,N-dimethylformamide, N,N-dimethylacetamide, and the like) and mixtures thereof. Optionally, 1-hydroxybenzotriazole (HOBT), HOBT-6-carboxaamidomethyl polystyrene, or 1-hydroxy-7-azabenzotriazole (HOAT) can be used as a reactant agent. Suitable reaction temperatures range from about −20° C. to 50° C., and about 0° C. to 40° C.

Alternatively, Amide Q can be obtained by an amidation reaction using an acid halide (such as, ArCOCl) and a base in an inert solvent. The base includes a tertiary amine (such as, N,N-diisopropylethylamine, triethylamine, N-methylmorpholine, and the like), or an aromatic amine (such as, pyridine, imidazole, poly-(4-vinylpyridine), and the like). The inert solvent includes lower halocarbon solvents (such as, dichloromethane, dichloroethane, chloroform, and the like), ethereal solvents (such as, tetrahydrofuran, dioxane, and the like), amide solvents (such as, N,N-dimethylformamide, and the like), aromatic solvents (benzene, toluene, pyridine, and the like) and mixtures thereof. Suitable reaction temperatures range from about −20° C. to 50° C., and about 0° C. to 40° C.

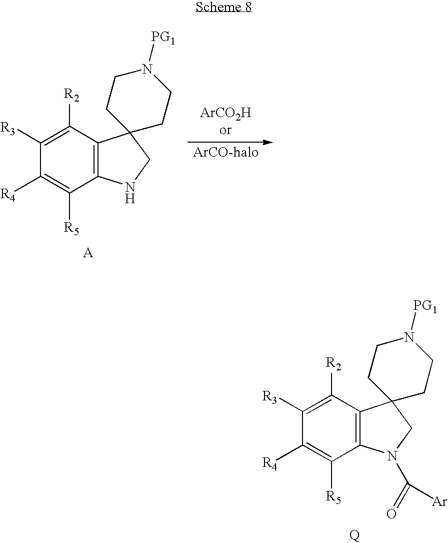

Illustrated in Scheme 9 is a method useful in the preparation of the sulfonamide group. Sulfonamide R can be obtained from common intermediate Indole A by a sulfonation reaction using ArSO₂halo (such as ArSO₂Cl, and the like) in an inert solvent with or without a base. When present, suitable bases include tertiary amines (such as, N,N-diisopropylethylamine, triethylamine, N-methylmorpholine, and the like), aromatic amine (such as, pyridine, imidazole, poly-(4-vinylpyridine)), and the like. Suitable inert solvents include lower halocarbon solvents (such as, dichloromethane, dichloroethane, chloroform, and the like), ethereal solvents (such as, tetrahydrofuran, dioxane, and the like), aromatic solvents (such as, benzene, toluene, and the like), or polar solvents (such as, N,N-dimethylformamide, dimethyl sulfoxide, and the like). Suitable reaction temperatures range from about −20° C. to 120° C., and about 0° C. to 100° C.

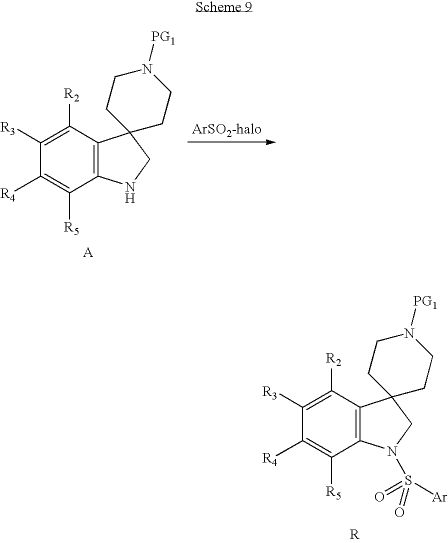

Alternatively, Indole L can be functionalized while PG₂ is still present. The R₁ group can be introduced using a variety of methods know in the art. Some of the methods are illustrated in Schemes 10 and 11.

As shown in Schemes 10 and 11, Indole L can be functionalized with the R₁ group via a number of different methods, such as, reduction of an amide, reductive amination and direct alkylation.

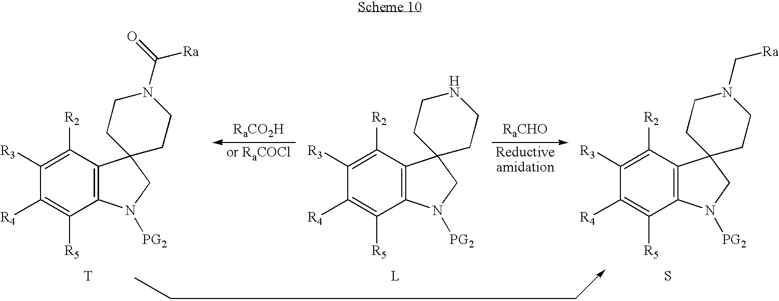

As shown in Scheme 10, Indole L can be functionalized while PG₂ is still present. For example, Indole L can be reacted with a carboxylic acid (RₐCO₂H, wherein Rₐ is CH₃CH₂— optionally substituted with 1, 2, 3, 4, or 5 fluorine atoms, examples of Rₐ include, CF₃CH₂—, CHF₂CH₂—, CF₃CF₂—, and the like) with a dehydrating condensing agent in an inert solvent with or without a base to provide Amide T. Suitable dehydrating condensing agents include dicyclohexylcarbodiimide (DCC), 1,3-diisopropylcarbodiimide (DIC), 1-ethyl-3-(3-dimethylaminopropyl)carbodiimide hydrochloride (EDC.HCl), bromo-tris-pyrrolidino-phosphonium hexafluorophosphate (PyBroP), benzotriazoloyloxytris(dimethylamino)-phosphonium hexafluorophosphate (BOP), O-(7-azabenzo triazol-1-yl)-1,1,3,3-tetramethyluronium hexafluorophosphate (HATU), or 1-cyclohexyl-3-methylpolystyrene-carbodiimide. The base includes a tertiary amine (such as, N,N-diisopropylethylamine, triethylamine, and the like). Inert solvents include lower halocarbon solvents (such as, dichloromethane, dichloroethane, chloroform, and the like), ethereal solvents (such as, tetrahydrofuran, dioxane, and the like), nitrile solvents (such as, acetonitrile, and the like), amide solvents (N,N-dimethylformamide, N,N-dimethylacetamide, and the like) and mixtures thereof. Optionally, 1-hydroxybenzotriazole (HOBT), HOBT-6-carboxaamidomethyl polystyrene, or 1-hydroxy-7-azabenzotriazole (HOAT) can be used as a reactant agent. Suitable reaction temperatures range from about −20° C. to 50° C., and about 0° C. to 40° C. Alternatively, Amide T can be obtained by an amidation reaction using an acid halide (such as, RₐCOCl) and a base in an inert solvent in a similar manner as described herein.

In a subsequent step, Amide T can be reduced to give Amine S thus providing R₁ groups having the formula RₐCH₂—, such as, R₁ is CF₃CH₂CH₂—, CHF₂CH₂CH₂—, CF₃CF₂CH₂—, and the like. Suitable reducing agents include alkali metal aluminum hydrides (such as, lithium aluminum hydride, and the like), alkali metal borohydrides (such as, lithium borohydride, and the like), alkali metal trialkoxyaluminum hydrides (such as, lithium tri-tert-butoxyaluminum hydride, and the like), dialkylaluminum hydrides (such as, di-isobutylaluminum hydride, and the like), borane, dialkylboranes (such as, di-isoamyl borane, and the like), alkali metal trialkylboron hydrides (such as, lithium triethylboron hydride, and the like). Inert solvents include ethereal solvents (such as, tetrahydrofuran, dioxane, and the like), aromatic solvents (such as, toluene, and the like) and mixtures thereof. Suitable reaction temperatures range from about −78° C. to 200° C., and about 50° C. to 120° C.

Alternatively, Amine S can be obtained by a reductive amination reaction from common intermediate Indole A with an aldehyde (RₐCHO, wherein Rₐ has the same definition as described herein) and a reducing agent in an inert solvent with or without an acid. Reducing agents include sodium triacetoxyborohydride, sodium cyanoborohydride, sodium borohydride, borane-pyridine complex, and the like. Inert solvents include lower alkyl alcohol solvents (such as, methanol, ethanol, and the like), lower halocarbon solvents (such as, dichloromethane, dichloroethane, chloroform, and the like), ethereal solvents (such as, tetrahydrofuran, dioxane, and the like), aromatic solvents (such as, benzene, toluene, and the like) and mixtures thereof. Suitable acids include inorganic acid (such as, hydrochloric acid, sulfuric acid, and the like) or organic acid (such as, acetic acid, and the like). Suitable reaction temperatures range from about −20° C. to 120° C., and about 0° C. to 100° C. In addition, this reaction can optionally be carried out under microwave conditions.

An alternative method is illustrated in Scheme 11, Indole L can be directly alkylated with an alkylating agent, such as R₁-LG (wherein R₁ has the same meaning as described herein, and LG is a leaving group, such as, chloro, bromo, iodo, OMs, OTs, OTf and the like), in the presence of a base and in an inert solvent to provide Amine U. Suitable bases include alkali metal carbonate (such as, sodium carbonate, potassium carbonate, and the like), alkali metal hydride (such as, sodium hydride, potassium hydride, and the like), alkali metal alkoxide (such as, potassium tert-butoxide, sodium tert-butoxide, and the like); alkyl lithiums (such as, tert-butyl lithium, n-butyl lithium and the like). Inert solvents include ethereal solvents (such as, tetrahydrofuran, dioxane), aromatic solvents (such as, benzene, toluene, and the like), amide solvents (such as, N,N-dimethylformamide, and the like) and mixtures thereof. Suitable reaction temperatures range from about −20° C. to 120° C., preferably about 0° C. to 100° C.

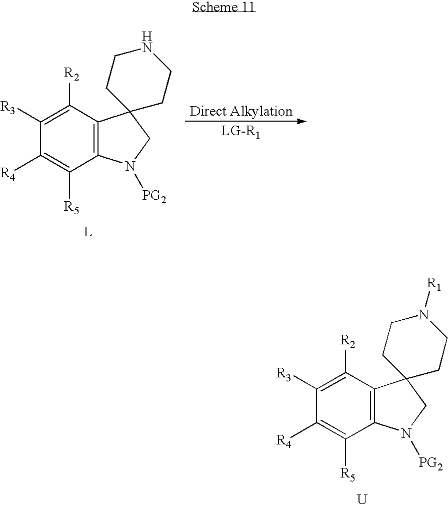

As illustrated in Scheme 12, the protecting group for the monoprotected intermediate Indole U (e.g., —PG₂) can be removed using conditions known in the art to give Indole X. The resulting nitrogen can be modified using similar procedures as described above to provide compounds of the invention.

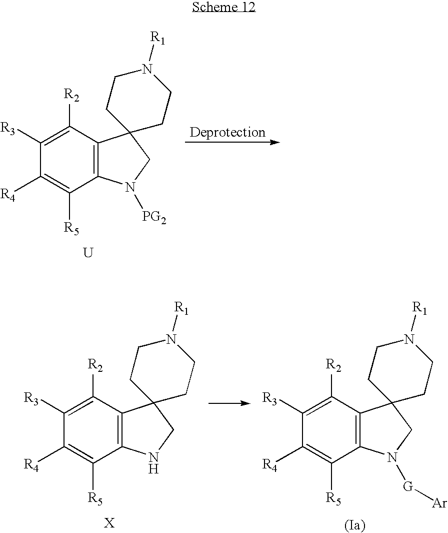

Similarly, as illustrated in Scheme 13, the protecting group for the monoprotected intermediate Indole W (e.g., —PG₁) can be removed using conditions known in the art to give Indole Y. The resulting nitrogen can be modified using similar procedures as described above to provide compounds of the invention.

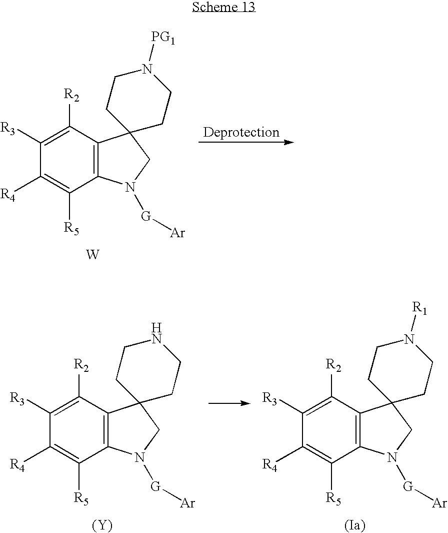

Synthetic methods for incorporating isotopes or radio-isotopes into organic compounds are applicable to the compounds of the invention and are well known in the art. Synthetic methods for incorporating activity levels of tritium into target molecules are as follows:

A. Catalytic Reduction with Tritium Gas—This procedure normally yields high specific activity products and requires halogenated or unsaturated precursors.

B. Reduction with Sodium Borohydride [³H]—This procedure is rather inexpensive and requires precursors containing reducible functional groups such as aldehydes, ketones, lactones, esters, and the like.

C. Reduction with Lithium Aluminum Hydride [³H]—This procedure offers products at almost theoretical specific activities. It also requires precursors containing reducible functional groups such as aldehydes, ketones, lactones, esters, and the like.

D. Tritium Gas Exposure Labeling—This procedure involves exposing precursors containing exchangeable protons to tritium gas in the presence of a suitable catalyst.

E. N-Methylation using Methyl Iodide [³H]—This procedure is usually employed to prepare O-methyl or N-methyl [³H] products by treating appropriate precursors with high specific activity methyl iodide [³H]. This method in general allows for higher specific activity, such as for example, about 70-90 Ci/mmol.

Synthetic methods for incorporating activity levels of ¹²⁵I into target molecules include:

A. Sandmeyer and like reactions—This procedure transforms an aryl or heteroaryl amine into a diazonium salt, such as a tetrafluoroborate salt, and subsequently to ¹²⁵I labeled compound using Na¹²⁵I. A represented procedure is found in Zhu, D.-G. et al., J. Org. Chem. 67, 943-948 (2002).

B. Ortho ¹²⁵Iodination of phenols—This procedure allows for the incorporation of ¹²⁵I at the ortho position of a phenol as reported by Collier, T. L. et al., J. Labeled Compd Radiopharm. 42, S264-S266 (1999).

C. Aryl and heteroaryl bromide exchange with ¹²⁵I—This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A represented procedure was reported by Bas, M.-D. et al., J. Labeled Compd Radiopharm. 44, S280-S282 (2001).

Certain Compounds of the Invention can have asymmetric centers and therefore exist in different enantiomeric and diastereomeric forms. A Compound of the Invention can be in the form of an optical isomer or a diastereomer. Accordingly, the invention encompasses Compounds of the Invention and their uses as described herein in the form of their optical isomers, diasteriomers and mixtures thereof, including a racemic mixture. Optical isomers of the Compounds of the Invention can be obtained by known techniques such as chiral chromatography or formation of diastereomeric salts from an optically active acid or base.

In addition, one or more hydrogen, carbon or other atoms of a Compound of the Invention can be replaced by an isotope of the hydrogen, carbon or other atoms. Such compounds, which are encompassed by the present invention, are useful as research and diagnostic tools as well as in Mas receptor binding assays.

### Methods

One aspect of the present invention pertains to methods for inhibiting Mas function in a cell comprising contacting a cell capable of expressing Mas with an amount of a Compound of the Invention effective to inhibit Mas function in the cell. This method can be used in vitro, for example, as an assay to select cells that express Mas and, accordingly, is useful as part of an assay to select compounds useful for treating, preventing and/or managing a vascular or cardiovascular disease or disorder or a neurological disease or disorder. The method is also useful for inhibiting Mas function in a cell in vivo, such as in a patient, in a human in one embodiment, by contacting a cell, in a patient, with an amount of a Compound of the Invention effective to inhibit Mas function in the cell.

One aspect of the present invention pertains to methods for treating or preventing a vascular or cardiovascular disease or disorder comprising administering to a patient in need thereof an effective amount of a Compound of the Invention or a pharmaceutical composition thereof. In some embodiments, the compound of the present invention does not significantly increase blood pressure. In some embodiments, the vascular or cardiovascular disorder is atherosclerosis, reperfusion injury, acute myocardial infarction, high blood pressure, primary or secondary hypertension, renal vascular hypertension, acute or chronic congestive heart failure, left ventricular hypertrophy, vascular hypertrophy, glaucoma, primary or secondary hyperaldosteronism, diabetic nephropathy, glomerulonephritis, scleroderma, glomerular sclerosis, renal failure, renal transplant therapy, diabetic retinopathy or migraine.

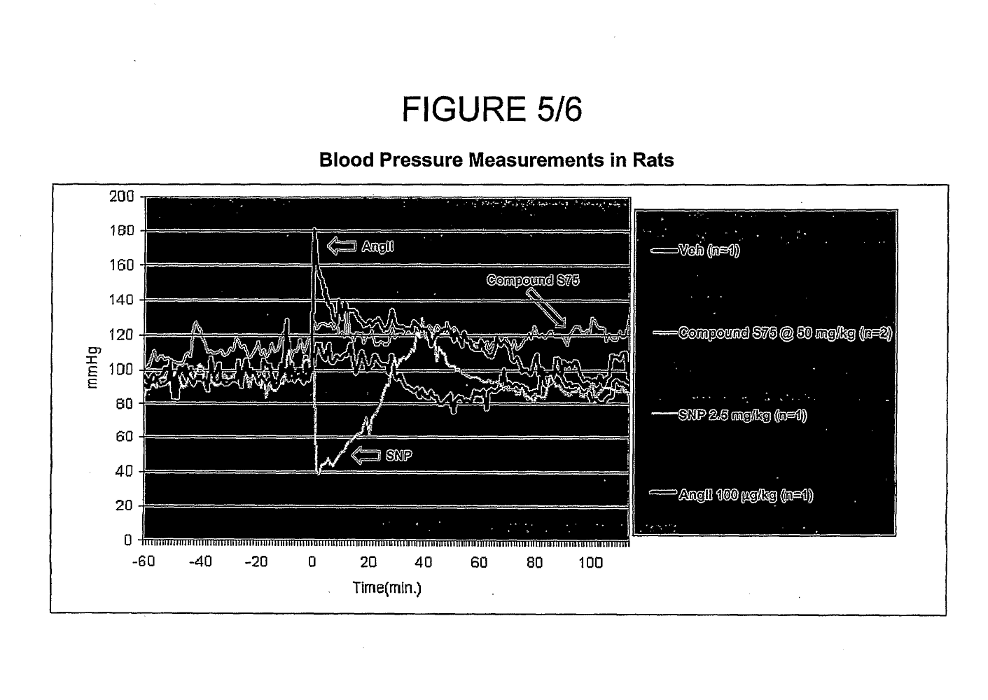

A significant increase in blood pressure is the increase in blood pressure that would be observed after treatment with a known vasoconstrictor compound. An example of a significant increase in blood pressure is shown in FIG. 5. In FIG. 5, the known vasoconstrictor angiotensin II was administered to rats and a significant increase in blood pressure was recorded after administration. A significant increase in blood pressure can be, for example, an increase in blood pressure of 10% or more, 15% or more, 20% or more, 30% or more, 40% or more, 50% or more, 60% or more, 70% or more, 80% or more, 90% or more, or 100% or more. As understood by one skilled in the art, blood pressure readings can be increased in response to factors other than administration of a compound, such as stress. Therefore, care should be taken to control for these other factors.

One aspect of the present invention pertains to methods for treating or preventing a neurological disease or disorder comprising administering to a patient in need thereof an effective amount of a Compound of the Invention or a pharmaceutical composition thereof. In some embodiments, the neurological disease or disorder is diabetic peripheral neuropathy, pain, stroke, cerebral ischemia or Parkinson's disease.

One aspect of the present invention pertains to methods for treating or preventing a disease or disorder treatable or preventable by inhibiting Mas receptor function, comprising administering to a patient in need thereof an effective amount of a Compound of the Invention or a pharmaceutical composition thereof. In some embodiments, the disease or disorder is a vascular or cardiovascular disease or disorder. In some embodiments, the vascular or cardiovascular disease or disorder is atherosclerosis, reperfusion injury, acute myocardial infarction, high blood pressure, primary or secondary hypertension, renal vascular hypertension, acute or chronic congestive heart failure, left ventricular hypertrophy, vascular hypertrophy, glaucoma, primary or secondary hyperaldosteronism, diabetic nephropathy, glomerulonephritis, scleroderma, glomerular sclerosis, renal failure, renal transplant therapy, diabetic retinopathy or migraine. In some embodiments, the disease or disorder is a neurological disease or disorder. In some embodiments, the neurological disease or disorder is diabetic peripheral neuropathy, pain, stroke, cerebral ischemia or Parkinson's disease.

One aspect of the present invention pertains to use of a compound of the present invention for the manufacture of a medicament comprising a Compound of the Invention for use in the treatment of a vascular or cardiovascular disease.

One aspect of the present invention pertains to use of a compound of the present invention for the manufacture of a medicament comprising a Compound of the Invention for use in the treatment of a neurological disease.

One aspect of the present invention pertains to use of a compound of the present invention for the manufacture of a medicament comprising a Compound of the Invention for use as a neuro-protective agent.

One aspect of the present invention pertains to use of a compound of the present invention for the manufacture of a medicament comprising a Compound of the Invention for use as a cardio-protective agent.

One aspect of the present invention pertains to use of a compound of the present invention for use in a method of treatment of the human or mammal body by therapy.

One aspect of the present invention pertains to use of a compound of the present invention for use in a method of treatment of a vascular or cardiovascular disease of the human or mammal body by therapy.

One aspect of the present invention pertains to use of a compound of the present invention for use in a method of treatment of a neurological disease or disorder of the human or mammal body by therapy.

One aspect of the present invention pertains to methods for producing a pharmaceutical composition comprising admixing one or more Compound(s) of the Invention and a pharmaceutically acceptable vehicle or excipient.

In some embodiments, the compound is orally bioavailable. In some embodiments, said oral bioavailability can be shown to be at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% relative to intraperitoneal administration.

The invention further relates to a method for inhibiting Mas receptor function in a cell, comprising contacting a cell capable of expressing Mas with an effective amount of a Compound of the Invention.

The invention also relates to a method for inhibiting Mas receptor activity in a human host, comprising administering a compound that inhibits activity of the Mas receptor gene product to a human host in need of such treatment. For example, the invention relates to a method for selectively inhibiting Mas receptor activity in a human host, comprising administering a Compound of the Invention that selectively inhibits activity of the Mas receptor gene product to a human host in need of such treatment. Further, for example, the invention relates to a method for selectively inhibiting Mas receptor activity in a human host, comprising administering an inverse agonist of the Mas receptor that selectively inhibits activity of the Mas receptor gene product to a human host in need of such treatment. Selectively inhibiting Mas receptor activity means significantly inhibiting Mas receptor activity while not significantly inhibiting the activity of, for example, one or more other GPCR, a majority of other GPCRs, or any other GPCR.

The invention further relates to a method for selectively inhibiting Mas receptor activity in a human host, comprising administering a Compound of the Invention or a pharmaceutically acceptable salt, free base, solvate, hydrate or stereoisomer thereof, as described herein, that selectively inhibits activity of the Mas receptor gene product to a human host in need of such treatment.

The invention further relates to a method for effecting cardio protection in an individual in need of said cardio protection, comprising administering to said individual an effective amount of a Compound of the Invention or a pharmaceutical composition thereof.

The invention also relates to a method for treating or preventing a vascular or cardiovascular disease or disorder in an individual in need of said treating or preventing, comprising administering to said individual an effective amount of a Compound of the Invention or a pharmaceutical composition thereof.

In one embodiment, the pharmaceutical compositions of the invention are used alone for treating or preventing a disease or disorder. In another embodiment, the pharmaceutical compositions of the invention are used in combination with another compound or therapy for treating or preventing a disease or disorder.

As understood by one skilled in the art, a vascular disease or disorder is a disease or disorder related to blood vessels in an animal and a cardiovascular disease or disorder is a disease or disorder related to the heart or blood vessels. Thus, a cardiovascular disease can be considered as a subset of vascular diseases. A neurological disease or disorder is a disease or disorder related to the nervous system in an animal. Some diseases such as stroke and migraine can be considered as both a neurological disease and as a vascular disease.

In one embodiment, said vascular or cardiovascular disease or disorder is atherosclerosis, reperfusion injury, acute myocardial infarction, high blood pressure, primary or secondary hypertension, renal vascular hypertension, acute or chronic congestive heart failure, left ventricular hypertrophy, vascular hypertrophy, glaucoma, primary or secondary hyperaldosteronism, diabetic nephropathy, glomerulonephritis, scleroderma, glomerular sclerosis, renal failure, renal transplant therapy, diabetic retinopathy or migraine. In another embodiment, said vascular or cardiovascular disease or disorder is reperfusion injury, acute myocardial infarction, acute or chronic congestive heart failure, left ventricular hypertrophy or vascular hypertrophy.

In another embodiment, the Compounds of the Invention are useful as neuro-protective and/or cardio-protective agents and have the ability to prevent or lessen the severity of cerebral ischemia. In a certain embodiment, the cerebral ischemia results from stroke. Compounds of the Invention can prevent or lessen the severity of cerebral ischemia by preventing or lessening acute injury to ischemic neurons.

In another embodiment, the Compounds of the Invention are used in combination with, or in place of, angiotensin-converting enzyme (ACE) inhibitors to treat the diseases or disorders for which such ACE inhibitors are conventionally used. Such diseases or disorders include, but are not limited to, refractory hypertension, congestive heart failure, myocardial infarction, diabetes mellitus, chronic renal insufficiency, atherosclerotic cardiovascular disease, reinfarction, angina, end-stage renal disease, left ventricular dysfunction, or any disease or disorder associated with the renin-angiotensin system.

In one embodiment, an effective amount of a Compound of the Invention can be used to treat, prevent and/or manage any disease or disorder treatable, preventable and/or manageable by binding to the Mas receptor. Examples of diseases or disorders that are treatable or preventable by inhibiting binding to the Mas receptor include, but are not limited to, vascular, cardiovascular or neurological diseases or disorders. In a particular embodiment, an effective amount of a Compound of the Invention can be used to treat, prevent and/or manage any disease or disorder treatable, preventable and/or manageable by inhibiting Mas receptor function.

### Therapeutic Uses of the Compounds of the Invention

In accordance with the invention, the Compounds of the Invention are useful as cardio-protective and/or neuro-protective agents. The Compounds of the Invention can also be administered to a patient in need of treatment, prevention and/or management of a vascular or cardiovascular or neurological disease or disorder.

In one embodiment, the vascular or cardiovascular disease or disorder is atherosclerosis, reperfusion injury, acute myocardial infarction, high blood pressure, primary or secondary hypertension, renal vascular hypertension, acute or chronic congestive heart failure, left ventricular hypertrophy, vascular hypertrophy, glaucoma, primary or secondary hyperaldosteronism, diabetic neuropathy, glomerulonephritis, scleroderma, glomerular sclerosis, renal failure, renal transplant therapy, diabetic retinopathy, or another vascular disorders such as migraine.

In another embodiment, the neurological disease or disorder is diabetic peripheral neuropathy, pain, stroke, cerebral ischemia or Parlinson's disease.

In another embodiment, the Compounds of the Invention are useful as neuro-protective and/or cardio-protective agents and have the ability to prevent or lessen the severity of cerebral ischemia. In a certain embodiment, the cerebral ischemia results from stroke. Compounds of the Invention can prevent or lessen the severity of cerebral ischemia by preventing or lessening acute injury to ischemic neurons.

In another embodiment, the Compounds of the Invention are used in combination with, or in place of, angiotensin-converting enzyme (ACE) inhibitors to treat the diseases or disorders for which such ACE inhibitors are conventionally used. Such diseases or disorders include, but are not limited to, refractory hypertension, congestive heart failure, myocardial infarction, diabetes mellitus, chronic renal insufficiency, atherosclerotic cardiovascular disease, reinfarction, angina, end-stage renal disease, left ventricular dysfunction, or any disease or disorder associated with the renin-angiotensin system.

In one embodiment, an effective amount of a Compound of the Invention can be used to treat, prevent and/or manage any disease or disorder treatable, preventable and/or manageable by binding to the Mas receptor. Examples of diseases or disorders that are treatable or preventable by inhibiting binding to the Mas receptor include, but are not limited to, vascular, cardiovascular or neurological diseases or disorders. In a particular embodiment, an effective amount of a Compound of the Invention can be used to treat, prevent and/or manage any disease or disorder treatable, preventable and/or manageable by inhibiting Mas receptor function.

The invention further relates to methods for inhibiting Mas function in a cell comprising contacting a cell capable of expressing Mas with an amount of a Compound of the Invention effective to inhibit Mas function in the cell. This method can be used in vitro, for example, as an assay to select cells that express Mas and, accordingly, is useful as part of an assay to select compounds useful for treating, preventing and/or managing a vascular or cardiovascular disease or disorder or a neurological disease or disorder. The method is also useful for inhibiting Mas function in a cell in vivo, such as in a patient, in a human in one embodiment, by contacting a cell, in a patient, with an amount of a Compound of the Invention effective to inhibit Mas function in the cell.

### Therapeutic/Prophylactic Administration and Compositions of the Invention

Due to their activity, the Compounds of the Invention are advantageously useful in veterinary and human medicine. As described above, the Compounds of the Invention are useful for treating, preventing and/or managing a vascular or cardiovascular or neurological disease or disorder in a patient in need thereof. Accordingly, in one embodiment, the present invention relates to a method for manufacturing a medicament comprising one or more Compounds of the Invention and a pharmaceutically acceptable vehicle or excipient. In another embodiment, the medicament can further comprise another active agent.

When administered to a patient, the Compounds of the Invention can be administered as a component of a composition, such as a pharmaceutical composition, that comprises a pharmaceutically acceptable vehicle or excipient. The present compositions, which comprise a Compound of the Invention, can be administered intradermally, intramuscularly, intraperitoneally, intravenously, subcutaneously, intranasally, epidurally, orally, sublingually, intracerebrally, intravaginally, transdermally, rectally, by inhalation, topically (particularly to the ears, nose, eyes, or skin), by infusion or bolus injection, or by absorption through epithelial or mucocutaneous linings (e.g., oral, rectal, or intestinal mucosa) and can optionally be administered together with another active agent. Administration can be systemic or local. Various delivery systems are known, e.g., encapsulation in liposomes, microparticles, microcapsules or capsules, and can be used to administer the Compound of the Invention.

In specific embodiments, it can be desirable to administer the Compounds of the Invention locally. This can be achieved, for example, and not by way of limitation, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the Compounds of the Invention into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the Compounds of the Invention can be formulated as a suppository, with traditional binders and excipients such as triglycerides.

In another embodiment, the Compounds of the Invention can be delivered in a vesicle, in particular a liposome (See Langer, Science 249:1527-1533 (1990) and Treat et al., Liposomes in the Therapy of Infectious Disease and Cancer 317-327 and 353-365 (1989).

In yet another embodiment, the Compounds of the Invention can be delivered in a controlled-release system or sustained-release system (See, e.g., Goodson, in Medical Applications of Controlled Release, supra, vol. 2, pp. 115-138 (1984)). Other controlled- or sustained-release systems discussed in the review by Langer, Science 249:1527-1533 (1990) can be used. In one embodiment, a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)). In another embodiment, polymeric materials can be used (See Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61 (1983); Levy et al., Science 228:190 (1985); During et al., Ann. Neurol. 25:351 (1989); and Howard et al., J. Neurosurg. 71:105 (1989)). In yet another embodiment, a controlled- or sustained-release system can be placed in proximity of a target of the Compounds of the Invention, e.g., the spinal column, brain, or gastrointestinal tract, thus requiring only a fraction of the systemic dose.

The present pharmaceutical compositions can optionally comprise a suitable amount of a pharmaceutically acceptable excipient so as to provide the form for proper administration to the patient.

The pharmaceutical compositions can be for a single, one-time use or can contain antimicrobial excipients, as described herein, rendering the pharmaceutical compositions suitable for multiple uses, for example a multi-use vial. In another embodiment, the pharmaceutical compositions can be in unit dose or unit-of-use packages. As is known to those of skill in the art, a unit dose package provides delivery of a single dose of a drug to a subject. The methods of the invention provide for a unit dose package of a pharmaceutical composition comprising, for example, 700 mcg of a Compound of the Invention per unit. The 700 mcg of a Compound of the Invention, is an amount that administers 10 mcg/kg to a 70 kg subject, for example. The unit can be, for example, a single use vial, a pre-filled syringe, a single transdermal patch and the like.

As is known to those of skill in the art, a unit-of-use package is a convenient, prescription size, patient ready unit labeled for direct distribution by health care providers. A unit-of-use package contains a pharmaceutical composition in an amount necessary for a typical treatment interval and duration for a given indication. The methods of the invention provide for a unit-of-use package of a pharmaceutical composition comprising, for example, a Compound of the Invention in an effective amount for treating an average sized adult male or female. It will be apparent to those of skill in the art that the doses described herein are based on the subject's body weight.

The pharmaceutical compositions can be labeled and have accompanying labeling to identify the composition contained therein and other information useful to health care providers and subjects in the treatment of a vascular or cardiovascular or neurological disorder, including, but not limited to, instructions for use, dose, dosing interval, duration, indication, contraindications, warnings, precautions, handling and storage instructions and the like.

The term “label” refers to a display of written, printed or graphic matter upon the immediate container of an article, for example the written material displayed on a vial containing a pharmaceutically active agent.

The term “labeling” refers to all labels and other written, printed or graphic matter upon any article or any of its containers or wrappers or accompanying such article, for example, a package insert or instructional videotapes or DVDs accompanying or associated with a container of a pharmaceutically active agent.

Pharmaceutical excipients for use in the present pharmaceutical compositions can be liquids, such as water and oils, including those of petroleum, animal, vegetable, or synthetic origin, such as peanut oil, soybean oil, mineral oil, sesame oil and the like. The pharmaceutical excipients can be saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to an animal. Water, and in one embodiment physiological saline, is a particularly useful excipient when the piperazine Compound is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsions, tablets, pills, pellets, capsules, capsules containing liquids, powders, sustained-release formulations, suppositories, aerosols, sprays, suspensions, or any other form suitable for use. In one embodiment, the composition is in the form of a capsule (See, e.g., U.S. Pat. No. 5,698,155). Other examples of suitable pharmaceutical excipients are described in Remington's Pharmaceutical Sciences 1447-1676 (Alfonso R. Gennaro ed., 19th ed. 1995), incorporated herein by reference.

In one embodiment, the Compounds of the Invention are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs, for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the Compounds of the Invention can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lidocaine to lessen pain at the site of the injection. The ingredients can be supplied either separately or mixed together in unit dosage form, for example, as a dry lyophilized powder or water free concentrate in a hermetically sealed container such as an ampoule or sachette indicating the quantity of active agent. Where the Compounds of the Invention are to be administered by infusion, they can be dispensed, for example, with an infusion bottle containing sterile pharmaceutical grade water or saline. Where the Compounds of the Invention are administered by injection, an ampoule of sterile water for injection or saline can be provided so that the ingredients can be mixed prior to administration.

The Compounds of the Invention can be administered by controlled-release or sustained-release means or by delivery devices that are known to those skilled in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, each of which is incorporated herein by reference. Such dosage forms can be used to provide controlled- or sustained-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

Controlled- or sustained-release pharmaceutical compositions can have a common goal of improving drug therapy over that achieved by their non-controlled or non-sustained counterparts. In one embodiment, a controlled- or sustained-release composition comprises a minimal amount of a Compound of the Invention to treat or prevent a disease or disorder in a minimal amount of time. Advantages of controlled- or sustained-release compositions include extended activity of the drug, reduced dosage frequency, and increased patient compliance. In addition, controlled- or sustained-release compositions can favorably affect the time of onset of action or other characteristics, such as blood levels of the Compound of the Invention, and can thus reduce the occurrence of adverse side effects.

Controlled- or sustained-release compositions can initially release an amount of a Compound of the Invention that promptly produces the desired therapeutic or prophylactic effect, and gradually and continually release other amounts of the Compound of the Invention to maintain this level of therapeutic or prophylactic effect over an extended period of time. To maintain a constant level of the Compound of the Invention in the body, the Compound of the Invention can be released from the dosage form at a rate that will replace the amount of the Compound of the Invention being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

The amount of the Compound of the Invention that is effective in the treatment or prevention of a disease or disorder can be determined by standard clinical techniques. In addition, in vitro or in vivo assays can optionally be employed to help identify optimal dosage ranges. The precise dose to be employed will also depend on the route of administration and the seriousness of the disorder and can be decided according to the judgment of a practitioner and/or each patient's circumstances. Suitable effective dosage amounts, however, range from about 0.01 mg/kg of body weight to about 2500 mg/kg of body weight, although they are typically about 100 mg/kg of body weight or less. In one embodiment, the effective dosage amount ranges from about 0.01 milligrams to about 100 milligrams of a Compound of the Invention, in another embodiment, about 0.02 mg/kg of body weight to about 50 mg/kg of body weight, and in another embodiment, about 0.025 mg/kg of body weight to about 20 mg/kg of body weight. In one embodiment, an effective dosage amount is administered about every 4 h. In one embodiment, an effective dosage amount is administered about every 6 h. In one embodiment, an effective dosage amount is administered about every 8 h. In one embodiment, an effective dosage amount is administered about every 12 h. In another embodiment, an effective dosage amount is administered about every 24 h.

Where a cell capable of expressing Mas is contacted with a Compound of the Invention in vitro, the amount effective for inhibiting the Mas receptor function in a cell will typically range from about 0.01 μg/L to about 5 mg/L, in one embodiment, from about 0.01 μg/L to about 2.5 mg/L, in another embodiment, from about 0.01 μg/L to about 0.5 mg/L, and in another embodiment, from about 0.01 μg/L to about 0.25 mg/L of a solution or suspension of a pharmaceutically acceptable carrier or excipient. In one embodiment, the volume of solution or suspension comprising the Compound of the Invention is from about 0.01 μL to about 1 mL. In another embodiment, the volume of solution or suspension is about 200 μL.

The Compounds of the Invention can be assayed in vitro or in vivo for the desired therapeutic or prophylactic activity prior to use in a humans. Animal model systems can be used to demonstrate safety and efficacy in humans.

The present methods for treating or preventing a disease or disorder in a patient in need thereof can further comprise administering another therapeutic agent to a patient being administered a Compound of the Invention. In one embodiment, the other therapeutic agent is administered in an effective amount.

The present methods for inhibiting Mas receptor function in a cell capable of expressing a Mas receptor can further comprise contacting the cell with an effective amount of another therapeutic agent.

Effective amounts of the other therapeutic agents are known to those skilled in the art. However, it is within the skilled artisan's purview to determine the other therapeutic agent's optimal effective-amount range. In one embodiment of the invention, where another therapeutic agent is administered to an animal, the effective amount of the Compound of the Invention is less than its effective amount would be where the other therapeutic agent is not administered. In this case, without being bound by theory, it is believed that the Compounds of the Invention and the other therapeutic agent act synergistically to treat or prevent a vascular or cardiovascular or neurological disease or disorder.

The other therapeutic agents can be, but is not limited to, aspirin, nitrates (e.g. nitroglycerin), ACE inhibitors, beta-blockers, calcium channel blockers, statins, N-methyl-D-aspartate (NMDA) receptor antagonists, non-NMDA neuroprotective agents, free-radical scavengers, or any other agent useful for treating, preventing and/or managing a vascular or cardiovascular or neurological disorder or useful as a neuroprotective agent.

Examples of ACE inhibitors include, but are not limited to, trandolapril, benazepril, captopril, enalapril, fosinopril, lisinopril, moexipril, quinapril and ramipril.

Examples of beta-blockers include, but are not limited to, propranolol, verapamil, and divalproex.

Examples of calcium channel blockers include, but are not limited to, bepridil, clentiazem, diltiazem, fendiline, gallopamil, mibefradil, prenylamine, semotiadil, terodiline, verapamil, amlodipine, aranidipine, barnidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, fantofarone, and perhexyline.

Examples of NMDA receptor antagonists include, but are not limited to, selfotel, aptiganel and magnesium.

Examples of non-NMDA neuroprotective agents include, but are not limited to, nalmefene, lubeluzole and clomethiazole.

An example of a free-radical scavenger includes, but is not limited to, tirilizad.

Examples of useful therapeutic agents for treating or preventing Parkinson's disease include, but are not limited to, carbidopa/levodopa, pergolide, bromocriptine, ropinirole, pramipexole, entacapone, tolcapone, selegiline, amantadine, and trihexyphenidyl hydrochloride.

Examples of useful therapeutic agents for treating or preventing stroke include, but are not limited to, anticoagulants such as heparin, agents that break up clots such as streptokinase or tissue plasminogen activator, agents that reduce swelling such as mannitol or corticosteroids, and acetylsalicylic acid.

Examples of useful therapeutic agents for treating or preventing a migraine include, but are not limited to, sumatriptan, methysergide, ergotamine, caffeine and beta-blockers.

A Compound of the Invention and the other therapeutic agent(s) can act additively or, in one embodiment, synergistically. In one embodiment, a Compound of the Invention is administered concurrently with another therapeutic agent; for example, a composition comprising an effective amount of a Compound of the Invention, an effective amount of another therapeutic agent can be administered. Alternatively, a composition comprising an effective amount of a Compound of the Invention and a different composition comprising an effective amount of another therapeutic agent can be concurrently administered. In another embodiment, an effective amount of a Compound of the Invention is administered prior or subsequent to administration of an effective amount of another therapeutic agent. In this embodiment, the Compound of the Invention is administered while the other therapeutic agent exerts its therapeutic effect, or the other therapeutic agent is administered while the Compound of the Invention exerts its preventative or therapeutic effect for treating or preventing a vascular or cardiovascular or neurological disorder.

In another embodiment, the Compound of the Invention is administered in combination with surgery associated with a vascular or cardiovascular or neurological disorder. Examples of surgery associated with a vascular or cardiovascular disorder include, but are not limited to, open-heart surgery, closed-heart surgery, coronary artery bypass surgery, heart valve surgery or angioplasty.

### Further Uses of the Compounds of the Invention

The invention further relates to methods for assaying the ability of a Compound of the Invention to bind to a Mas receptor, comprising contacting a radio-labeled Compound of the Invention with a cell or tissue capable of expressing a Mas receptor.

Radio-labeled Compounds of the Invention including, but not limited to, those containing one or more ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I or ¹³¹I atoms. The radionuclide that is incorporated in the radio-labeled Compound of the Invention will depend on the specific application of that radio-labeled compound. For example, for in vitro Mas receptor labeling and competition assays, compounds that incorporate ³H, ¹⁴C, ⁸²Br, ¹²⁵I, ¹³¹I, or ³⁵S will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

Certain isotopically-labeled Compounds of the Invention are useful in compound and/or substrate tissue distribution assays. In certain embodiments, the Compounds of the Invention containing a ³H and/or ¹⁴C isotopes are useful in these studies. In other embodiments, substitution with heavier isotopes such as deuterium (i.e., ²H) can afford certain therapeutic advantages resulting from greater metabolic stability including, but not limited to, increased in vivo half-life or reduced dosage requirements. Isotopically labeled Compounds of the Invention can generally be prepared by synthetic procedures analogous to those disclosed herein, by substituting an isotopically labeled reagent for a non-isotopically labeled reagent. It should be understood that all of the atoms represented in the compounds of the invention can be either the most commonly occurring isotope of such atoms or the more scarce radio-isotope or non-radioactive isotope.

In one embodiment, the invention relates to screening assays useful for identifying and/or evaluating Mas receptor binding ability of test compounds comprising the use of a radio-labeled Compound of the Invention. In general terms, a test compound can be evaluated for its ability to reduce binding of the radio-labeled Compound of the Invention to a Mas receptor. Accordingly, the ability of a test compound to compete with the radio-labeled Compound of the Invention for the binding to the Mas receptor directly correlates to its Mas receptor binding affinity.

In another embodiment, the invention relates to assays useful for locating or quantitating Mas receptor in a tissue sample, comprising contacting the tissue sample with an effective amount of a radio-labeled Compound of the Invention.

The radio-labeled Compounds of the Invention bind to the Mas receptor. In one embodiment the radio-labeled Compound of the Invention has an IC₅₀ less than about 500 μM, in another embodiment the radio-labeled Compound of the Invention has an IC₅₀ less than about 100 μM, in yet another embodiment the radio-labeled Compound of the Invention has an IC₅₀ less than about 10 μM, in yet another embodiment the radio-labeled Compound of the Invention has an IC₅₀ less than about 1 μM, in yet another embodiment the radio-labeled Compound of the Invention has an IC₅₀ less than about 0.1 μM, in yet another embodiment the radio-labeled Compound of the Invention has an IC₅₀ less than about 10 nM, and in still yet another embodiment the radio-labeled Compound of the Invention has an IC₅₀ less than about 1 nM.

Other uses of the disclosed radio-labeled Compounds of the Invention and methods will become apparent to those in the art based upon, inter alia, a review of this disclosure.

As will be recognized, the steps of the methods of the present invention need not be performed any particular number of times or in any particular sequence. Additional objects, advantages, and novel features of this invention will become apparent to those skilled in the art upon examination of the following examples thereof, which are intended to be illustrative and not intended to be limiting.

### Kits

The invention encompasses kits that can simplify the administration of a Compound of the Invention to a patient.

A typical kit of the invention comprises a unit dosage form of a Compound of the Invention. In one embodiment, the unit dosage form is a container, which can be sterile, containing an effective amount of a Compound of the Invention and a pharmaceutically acceptable vehicle or excipient. The kit can further comprise a label or printed instructions instructing the use of the Compound of the Invention. The kit can also further comprise a unit dosage form of another therapeutic agent, for example, a second container containing an effective amount of the other therapeutic agent and a pharmaceutically acceptable vehicle or excipient. In another embodiment, the kit comprises a container containing an effective amount of a Compound of the Invention, an effective amount of another therapeutic agent and a pharmaceutically acceptable vehicle or excipient. Examples of other therapeutic agents include, but are not limited to, those listed above.

Kits of the invention can further comprise a device that is useful for administering the unit dosage forms. Examples of such a device include but are not limited to a syringe, a drip bag, a patch, an inhaler, and an enema bag.

### EXAMPLES

The following examples are set forth to assist in understanding the invention and should not be construed as specifically limiting the invention described and claimed herein.

### Example 1

### Illustrative Compounds of the Invention

### Example 1.1

### Preparation of 1′-(propyl)-1,2-dihydro-5-tert-butyl-1-(2,6-difluoro-benzoyl)-spiro[3H-indole-3,4′-piperidine]trifluoroacetate (Compound 4 as the TFA salt)

### Step A: Synthesis of 1′-(benzyloxycarbonyl)-1,2-dihydro-5-t-butyl-spiro[3H-indole-3,4′-piperidine]

To a flask under N₂ containing 4-t-butylphenylhydrazine hydrochloride (1.0 g, 5 mmol) in degassed toluene/CH₃CN (50/1, 22 mL) and TFA (1.5 g, 13 mmol) was added 1-benzyloxycarbonyl-piperidine-4-carbaldehyde (1.2 g, 5 mmol) at room temperature. After stirring for 15 min, the reaction was heated to 40° C. and stirred overnight. The reaction was cooled to 5° C., and MeOH (27 mL) was added and followed by NaBH₄ (0.25 g, 6.7 mmol). The reaction was stirred for an additional 1 h, diluted with EtOAc (60 mL), and washed with 1N NaOH (2×) and brine. The organic layer was dried with anhydrous MgSO₄ and concentrated to afford solid residue, which was washed with hexane/ether (5/1) several times to give 1.0 g (51%) of 1′-(benzyloxycarbonyl)-1,2-dihydro-5-t-butyl-spiro[3H-indole-3,4′-piperidine].

LC-MS m/z 379 (M+H)⁺; ¹H NMR (400 MHz, DMSO-d₆) δ 7.39˜7.31 (m, 5H), 7.01 (s, 1H), 6.93 (d, 1H, J=7.8 Hz), 6.41 (d, 1H, J=7.8 Hz), 5.34 (s, 1H), 5.10 (s, 2H), 3.98 (bd, 2H, J=13 Hz), 3.37 (s, 2H), 2.98 (bm, 2H), 1.71˜1.55 (m, 4H), 1.21 (s, 9H).

### Step B: Synthesis of 1′-(benzyloxycarbonyl)-1,2-dihydro-5-t-butyl-1-(2,6-difluorobenzoyl)-spiro[3H-indole-3,4′-piperidine]

To a solution of 1′-(benzyloxycarbonyl)-1,2-dihydro-5-t-butyl-spiro[3H-indole-3,4′-piperidine] (1.75 g, 4.6 mmol) and Et₃N (1.5 mL, 12 mmol) in DCM (40 mL) at room temperature was added 2,6-difluorobenzoyl chloride (1.2 g, 6.9 mmol). After stirring for 3 h, the reaction was washed with sat. NaHCO₃ and water, dried with anhydrous MgSO₄, and concentrated. The crude product was purified by column chromatography (silica gel, hexane:EtOAc=95:5 to 70:30). 2.1 g (88%) of 1′-(benzyloxycarbonyl)-1,2-dihydro-5-t-butyl-1-(2,6-difluorobenzoyl)-spiro[3H-indole-3,4′-piperidine] was isolated.

LC-MS m/z 519 (M+H)⁺; ¹H NMR (400 MHz, CDCl₃) a mixture of two conformational isomers (2.5:1): major δ 8.25 (d, 1H, J=8.2 Hz), 7.46˜7.30 (m, 7H), 7.16 (s, 1H), 7.01 (t, 2H, J=7.8 Hz), 5.14 (s, 2H), 4.23 (bs, 2H), 3.76 (s, 2H), 2.79 (bs, 2H), 1.91˜1.67 (m, 4H), 1.32 (s, 9H). minor δ 7.46˜7.30 (m, 7H), 7.13 (s, 1H), 7.01 (m, 1H), 6.93 (d, 1H, J=8.4 Hz), 5.92 (d, 1H, J=8.4 Hz), 5.19 (s, 2H), 4.25 (bs, 2H), 4.24 (s, 2H), 3.02 (bs, 2H), 1.91˜1.67 (m, 4H), 1.24 (s, 9H).

### Step C: Synthesis of 1,2-dihydro-5-t-butyl-1-(2,6-difluorobenzoyl)-spiro[3H-indole-3,4′-piperidine]

A heterogenous solution of 1′-(benzyloxycarbonyl)-1,2-dihydro-5-t-butyl-1-(2,6-difluorobenzoyl)-spiro[3H-indole-3,4′-piperidine] (2 g, 4 mmol) and 10%-Pd—C (0.2 g, 5 mol %) in MeOH (80 mL) was shaken under hydrogen atmosphere (60 psi) at room temperature for 5 h. The reaction was filtered through a pad of Celite and concentrated. The oily residue was purified by column chromatography (silica gel, DCM:MeOH=100:0 to 90:10). 0.9 g (62%) of 1,2-dihydro-5-t-butyl-1-(2,6-difluorobenzoyl)-spiro[3H-indole-3,4′-piperidine] was isolated.

LC-MS m/z 385 (M+H)⁺; ¹H NMR (400 MHz, DMSO-d₆) a mixture of two conformational isomers (3:1): major δ 8.25 (d, 1H, J=8.2 Hz), 7.64 (m, 1H), 7.35˜7.30 (m, 4H), 3.73 (s, 2H), 2.85 (d, 2H, J=12 Hz), 2.38 (t, 2H, J=12 Hz), 1.76 (m, 2H), 1.50 (m, 2H), 1.30 (s, 9H), NH was not observed. minor δ 7.66 (m, 1H), 7.35˜7.30 (m, 3H), 6.96 (d, 1H, J=8.4 Hz), 5.77 (d, 1H, J=8.4 Hz), 4.11 (s, 2H), 2.93 (d, 2H, J=12 Hz), 2.63 (t, 2H, J=12 Hz), 1.76 (m, 2H), 1.50 (m, 2H), 1.21 (s, 9H), NH was not observed

### Step D: Synthesis of 1′-(propyl)-1,2-dihydro-5-tert-butyl-1-(2,6-difluoro-benzoyl)-spiro[3H-indole-3,4′-piperidine]trifluoroacetate

A sealed tube containing 1,2-dihydro-5-t-butyl-1-(2,6-difluorobenzoyl)-spiro[3H-indole-3,4′-piperidine] (30 mg, 0.08 mmol), 1-bromopropane (14 mg, 0.12 mmol), Et₃N (25 ul, 0.2 mmol), and DMF (1 mL) was reacted at 150° C. for 20 min in a microwave synthesizer. The reaction was directly purified by prep-HPLC, and dried under a lyophylizer to afford 25 mg (59%) of 1′-(propyl)-1,2-dihydro-5-tert-butyl-1-(2,6-difluoro-benzoyl)-spiro[3H-indole-3,4′-piperidine]trifluoroacetate.

LC-MS m/z 427 (M+H)⁺; ¹H NMR (400 MHz, DMSO-d₆) a mixture of two conformational isomers (3:1): major δ 9.31 (bs, 1H), 8.07 (d, 1H, J=8.6 Hz), 7.65 (m, 1H), 7.39 (dd, 1H, J=8.6 and 2.0 Hz), 7.32 (t, 2H, J=8.6 Hz), 7.21 (d, 1H, J=2.0 Hz), 3.89 (s, 2H), 3.45 (d, 2H, J=12 Hz), 2.99 (m, 4H), 2.15 (m, 2H), 1.88 (m, 2H), 1.66 (m, 2H), 1.30 (s, 9H), 0.90 (t, 3H, J=7.6 Hz). minor δ 9.31 (bs, 1H), 7.66 (m, 1H), 7.32 (m, 2H), 7.19 (d, 1H, J=2.0 Hz), 7.06 (dd, 1H, J=8.6 and 2.0 Hz), 5.81 (d, 1H, J=8.6 Hz), 4.26 (s, 2H), 3.26˜3.08 (m, 6H), 2.15 (m, 2H), 1.88 (m, 2H), 1.68 (m, 2H), 1.21 (s, 9H), 0.95 (t, 3H, J=7.6 Hz).

### Example 1.2

### Preparation of 1′-(3,3,3-trifluoro-propyl)-1,2-dihydro-5-tert-butyl-1-(2,6-difluoro-benzoyl)-spiro[3H-indole-3,4′-piperidine]trifluoroacetate (Compound 2 as the TFA salt)

### Step A: Synthesis of 1′-(3,3,3-trifluoro-propyl)-1,2-dihydro-5-tert-butyl-1-(2,6-difluoro-benzoyl)-spiro[3H-indole-3,4′-piperidine]trifluoroacetate

Using a similar procedure as described in Step D of Example 1, 1′-(3,3,3-trifluoropropyl)-1,2-dihydro-5-t-butyl-1-(2,6-difluorobenzoyl)-spiro[3H-indole-3,4′-piperidine]trifluoroacetate was isolated.

LC-MS m/z 481 (M+H)⁺; ¹H NMR (400 MHz, DMSO-d₆) a mixture of two conformational isomers (3:1): major δ 9.85 (bs, 1H), 8.07 (d, 1H, J=8.6 Hz), 7.66 (m, 1H), 7.39 (dd, 1H, J=8.4 and 2.0 Hz), 7.32 (m, 2H), 7.21 (s, 1H), 3.88 (s, 2H), 3.51 (m, 2H), 3.42 (m, 2H), 3.10˜2.82 (m, 4H), 2.11 (m, 2H), 1.91 (m, 2H), 1.30 (s, 9H). minor δ 9.85 (bs, 1H), 7.68 (m, 1H), 7.32 (m, 2H), 7.20 (s, 1H), 7.06 (dd, 1H, J=8.4 and 2.0 Hz), 5.81 (d, 1H, J=8.4 Hz), 4.25 (s, 2H), 3.60 (m, 2H), 3.42 (2H, overlapped with H₂O), 3.10˜2.82 (m, 4H), 2.11 (m, 2H), 1.91 (m, 2H), 1.21 (s, 9H).

### Example 1.3

### Preparation of 1′-(propyl)-1,2-dihydro-5-isopropyl-1-(2,6-difluoro-benzoyl)-spiro[3H-indole-3,4′-piperidine]trifluoroacetate (Compound 3 as the TFA salt)

### Step A: Synthesis of 1,2-dihydro-5-isopropyl-1-(2,6-difluorobenzoyl)-spiro[3H-indole-3,4′-piperidine]

Using a similar procedure as described in Step C of Example 1, the title compound was obtained.

LC-MS m/z 371 (M+H)⁺; ¹H NMR (400 MHz, DMSO-d₆) a mixture of two conformational isomers (3:1): major δ 9.05 (bs, 1H), 8.06 (d, 1H, J=8.2 Hz), 7.64 (m, 1H), 7.30 (t, 2H, J=8.0 Hz), 7.22 (dd, 1H, J=8.2 and 1.7 Hz), 7.10 (d, 1H, J=1.7 Hz), 3.87 (s, 2H), 3.21 (d, 2H, J=12 Hz), 2.96 (m, 2H), 2.93 (hept, 1H, J=6.8 Hz), 2.14 (m, 2H), 1.76 (m, 2H), 1.21 (d, 6H, J=6.8 Hz). minor δ 9.05 (bs, 1H), 7.66 (m, 1H), 7.32 (t, 2H, J=8.2 Hz), 7.09 (d, 1H, J=1.7 Hz), 6.89 (d, 1H, J=8.2 Hz), 5.80 (d, 1H, J=8.2 Hz), 4.24 (s, 2H), 3.32 (d, 2H, J=12 Hz), 3.13 (m, 2H), 2.82 (hept, 1H, J=6.8 Hz), 2.14 (m, 2H), 1.76 (m, 2H), 1.13 (d, 6H, J=6.8 Hz).

### Step B: Synthesis of 1′-(propyl)-1,2-dihydro-5-isopropyl-1-(2,6-difluoro-benzoyl)-spiro[3H-indole-3,4′-piperidine]trifluoroacetate

Using a similar procedure as described in Step D of Example 1, the title compound was obtained.

LC-MS m/z 413 (M+H)⁺; ¹H NMR (400 MHz, DMSO-d₆) a mixture of two conformational isomers (3:1): major δ 9.39 (bs, 1H), 8.07 (d, 1H, J=8.6 Hz), 7.65 (m, 1H), 7.32 (m, 2H), 7.23 (d, 1H, J=8.4 Hz), 7.08 (d, 1H, J=1.5 Hz), 3.89 (s, 2H), 3.45 (d, 2H, J=12 Hz), 2.98 (m, 4H), 2.93 (hept, 1H, J=6.8 Hz), 2.13 (m, 2H), 1.86 (m, 2H), 1.66 (m, 2H), 1.21 (d, 6H, J=6.8 Hz), 0.89 (t, 3H, J=7.2 Hz). minor δ 9.39 (bs, 1H), 7.66 (m, 1H), 7.32 (m, 2H), 7.07 (d, 1H, J=1.5 Hz), 6.90 (d, 1H, J=8.4 Hz), 5.81 (d, 1H, J=8.4 Hz), 4.25 (s, 2H), 3.54 (d, 2H, J=12 Hz), 3.20 (m, 2H), 3.09 (m, 2H), 2.82 (hept, 1H, J=6.8 Hz), 2.13 (m, 2H), 1.86 (m, 2H), 1.68 (m, 2H), 1.13 (d, 6H, J=6.8 Hz), 0.95 (t, 3H, J=7.2 Hz).

### Example 1.4

### Preparation of 1′-(3,3,3-trifluoro-propyl)-1,2-dihydro-5-isopropyl-1-(2,6-difluoro-benzoyl)-spiro[3H-indole-3,4′-piperidine]trifluoroacetate (Compound 1 as the TFA salt)

### Step A: Synthesis of 1′-(3,3,3-trifluoro-propyl)-1,2-dihydro-5-isopropyl-1-(2,6-difluoro-benzoyl)-spiro[3H-indole-3,4′-piperidine]trifluoroacetate

Using a similar procedure as described in Step D of Example 1, the title compound was obtained.

LC-MS m/z 467 (M+H)⁺; ¹H NMR (400 MHz, DMSO-d₆) a mixture of two conformational isomers (3:1): major δ 9.74 (bs, 1H), 8.07 (d, 1H, J=8.6 Hz), 7.66 (m, 1H), 7.32 (t, 2H, J=8.2 Hz), 7.23 (dd, 1H, J=8.4 and 1.8 Hz), 7.08 (s, 1H), 3.88 (s, 2H), 3.52 (m, 2H), 3.08˜2.81 (m, 6H), 2.93 (hept, 1H, J=7.2 Hz), 2.08 (m, 2H), 1.91 (m, 2H), 1.21 (d, 6H, J=7.2 Hz). minor δ 9.74 (bs, 1H), 7.67 (m, 1H), 7.32 (m, 2H), 7.08 (s, 1H), 6.90 (d, 1H, J=8.4 Hz), 5.81 (d, 1H, J=8.4 Hz), 4.25 (s, 2H), 3.54 (d, 2H, J=12 Hz), 3.08˜2.81 (m, 7H), 2.08 (m, 2H), 1.91 (m, 2H), 1.14 (d, 6H, J=7.2 Hz).

### Biological Assays

Several assays are well known in the art for determining whether a compound alters the functionality of a receptor, for example, the ability of a receptor to bind a ligand or other compound, or the ability of a receptor to initiate a signal transduction cascade. GPCR binding assays and functional assays are well known in the art (see, for example, “From Neuron To Brain” (3rd Ed.) Nichols, J. G. et al eds. Sinauer Assoicates, Inc. (1992)). For example, ligand binding assays, IP3 assays, cAMP assays, GPCR fusion protein assays, calcium flux assays, and GTPγS binding assays are well known in the art.

### Example 2

### Mas Receptor IP3 Assay

The Mas receptor IP3 assay was performed using a mammalian cell line (HEK293) which was transfected with a plasmid containing the human Mas receptor and selected for stable expression of the receptor. For the inverse agonist assay, higher levels of Mas receptor constitutive activity were desired. To achieve this, Mas receptor expression levels were increased by transiently transfecting the same Mas receptor stable cell line with additional human Mas receptor plasmid DNA following standard procedures. These cells were used in the Mas receptor IP3 assay approximately 24 hours post-transfection.

Cells were split into 96-well plates (50,000 cells/well) and allowed to attach for a period of 6 hours. The growth medium was then replaced with medium supplemented with 4 μCi/mL [3H]myo-inositol (100 μl; Perkin Elmer Life Sciences) and the cells were allowed to incubate for approximately 20 hours. Test compounds were serially diluted in inositol-free media containing 10 mM LiCl. The media in the plates was removed by aspiration, replaced with these test compound solutions and incubated at 37° C. for 1 hour.

Following this incubation, the media was removed by aspiration and replaced with buffer containing 0.1M formic acid. The plates were then frozen overnight at −80° C. to achieve complete cell lysis.

The following day, the assay plates were thawed at room temperature. The thawed contents were then transferred to 96-well filter plates (Nillipore, Multiscreen) pre-loaded with resin (Biorad, AG1-X8 100-200 mesh, formate form). The plate was filtered using a vacuum manifold and the resin was washed multiple times with water. An elution buffer was then applied (200 μl, 0.2M Ammonium formate/0.1M formic acid) and the resulting eluent was collected, under vacuum, in a 96-well collection plate. Aliquots of the eluent (80 μl) were transferred to filter plates (Whatman, Unifilter GF/C) and dried in a 45° C. oven overnight. Dried plates were counted on a scintillation counter following the addition of an appropriate scintillant (Perkin Elmer Life Sciences, Optiphase Supermix or Hi-Safe 3).

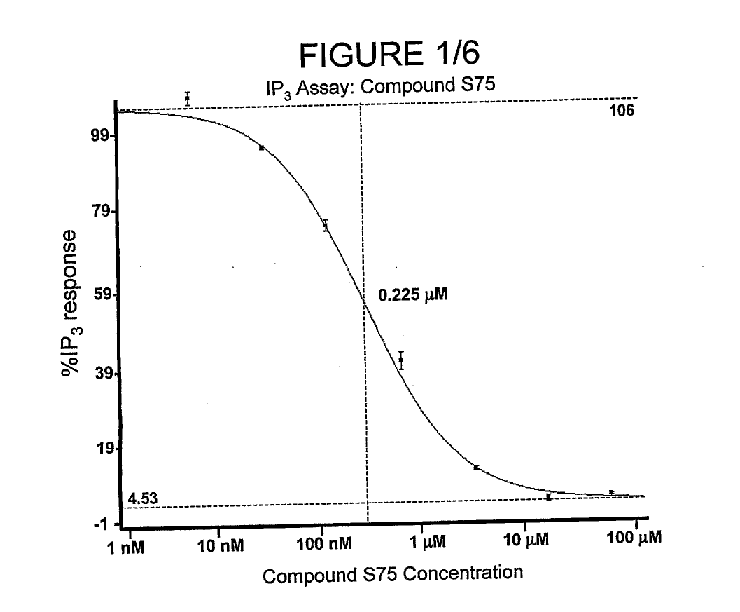

A representative experiment showing the results of an IP3 assay for Compound S75 is shown in FIG. 1. In this particular experiment, the IC₅₀ value for Compound S75 was 225 nM. The average IC₅₀ value for Compound S75 obtained from several experiments was 297.67 nM.

The IC₅₀ values of several Compounds of the Invention are listed in TABLE 5.

TABLE 5

| Cmpd No. | IC₅₀ (nM)* |
|---|---|
| 1 | 28.6 |
| 4 | 35.1 |

*n = 2

### Example 3

### Receptor Binding Assay

Several assays are well known in the art for identifying compounds that can bind to GPCRs. An example of a Mas receptor binding assay is described below.

Mas Receptor Preparation

293 cells (human kidney, ATCC), are transiently transfected with 10 μg human Mas receptor plasmid and 60 μl Lipofectamine (per 15-cm dish), grown in the dish for 24 hours (75% confluency) with a media change and removed with 10 mL/dish of Hepes-EDTA buffer (20 mM Hepes+10 mM EDTA, pH 7.4). The cells are then centrifuged in a Beckman Coulter centrifuge for 20 minutes, 17,000 rpm (JA-25.50 rotor). Subsequently, the pellet is resuspended in 20 mM Hepes+1 mM EDTA, pH 7.4 and homogenized with a 50-mL Dounce homogenizer and again centrifuged. After removing the supernatant, the pellets are stored at −80° C., until used in binding assay. When used in the binding assay, membranes are thawed on ice for about 20 minutes and then 10 mL of incubation buffer (20 mM Hepes, 1 mM MgCl2, 100 mM NaCl, pH 7.4) is added. The membranes are then vortexed to resuspend the crude membrane pellet and homogenized with a Brinkmann PT-3100 Polytron homogenizer for about 15 seconds at setting 6. The concentration of membrane protein is determined using the BRL Bradford protein assay.

Binding Assay

For total binding, a total volume of 50 μl of appropriately diluted membranes (diluted in assay buffer containing 50 mM Tris HCl (pH 7.4), 10 mM MgCl2, and 1 mM EDTA; 5-50 μg protein) is added to 96-well polypropylene microtiter plates followed by addition of 100 μl of assay buffer and 50 μl of a solution of a radiolabeled Compound of the Invention wherein the radiolabeled Compound of the Invention is present at a concentration of about 1 nM to 1 mM, preferably 1 nM to 500 μM, more preferably 1 nM to 100 μM, more preferably 10 nM to 100 μM, more preferably 100 nM to 100 μM, more preferably 1 μM to 100 μM and most preferably 10 μM to 100 μM. For nonspecific binding, 50 μl of assay buffer is added instead of 100 μl and an additional 50 μl of 10 μM cold Mas is added before 50 μl of a radiolabeled Compound of the Invention is added. Plates are then incubated at room temperature for about 60-120 minutes. The binding reaction is terminated by filtering assay plates through a Microplate Devices GF/C Unifilter filtration plate with a Brandell 96-well plate harvestor followed by washing with cold 50 mM Tris HCl, pH 7.4 containing 0.9% NaCl. The bottom of the filtration plate is then sealed, 50 μl of Optiphase Supermix is added to each well, the top of the filtration plates are sealed, and the filtration plates are counted in a Trilux MicroBeta scintillation counter. For compound competition studies, instead of adding 100 μl of assay buffer, 100 μl of appropriately diluted test compound is added to appropriate wells followed by addition of 50 μl of a radiolabeled Compound of the Invention.

### Example 4

### Ischemia-Reperfusion Injury in Isolated Adult Rat Hearts

Compounds of the invention can be characterized in several biological assays known in the art. For example, assays which analyze the effect of Compounds of the invention on the vascular, cardiovascular or nervous system can be performed. This example shows the results of assays which determine the effect of Compound S75 on ischemia-reperfusion injury in isolated adult rat hearts.

### Ischemia-Reperfusion Assay (Langendorff Apparatus):

Male Sprague-Dawley rats (300-350 g body weight) were anesthetized with Inactin (100 mg/kg IP) 20 minutes prior to surgery. The chest wall was opened and the heart was rapidly excised and immediately placed into ice-cold Krebs-Henseleit (KH) buffer (118 mM NaCl, 4.7 mM KCl, 1.2 mM MgSO₄, 1.2 mM KH₂PO₄, 1.5 nM CaCl₂, 25 mM NaHCO₃, 11 mM glucose, 1 mM pyruvate, and 0.005 mM EDTA) to produce cardiac arrest. The aorta was then cannulated and the heart retrogradely perfused with KH buffer maintained at 37° C. in a reservoir bubbled with 95% O₂/5% CO₂ (pH7.4) on the Langendorff apparatus at a constant pressure of 70 mmHg. Myocardial temperature was maintained at 37° C. by partially submerging the heart into a water-jacketed chamber filled with KH buffer. A water filled latex balloon attached to a metal cannula and inserted into the left ventricle via the mitral valve and connected to a pressure transducer (Powerlab, ADInstruments, Inc) was used for measurement of left ventricular pressure. The balloon was initially inflated to an end-diastolic pressure of 10 mmHg. After allowing 20 minutes for equilibration, rat hearts were subjected to 10 minutes of KH buffer containing drug or vehicle followed by 30 minutes of ischemia followed by 30 minutes of reperfusion. The difference between peak-systolic and end diastolic pressures, or left ventricular developed pressure (LVDP) was calculated as an index of contractile function and measured just prior to ischemia and at 10, and 30 minutes of reperfusion. Percent recovery of LV function [(LVDP post reperfusion/LVDP pre-ischemia)/100] was averaged across 5 vehicle and 4 drug treated hearts and one-way anova with Newman-Keuls Multiple Comparison Test was used to determine statistical significance.

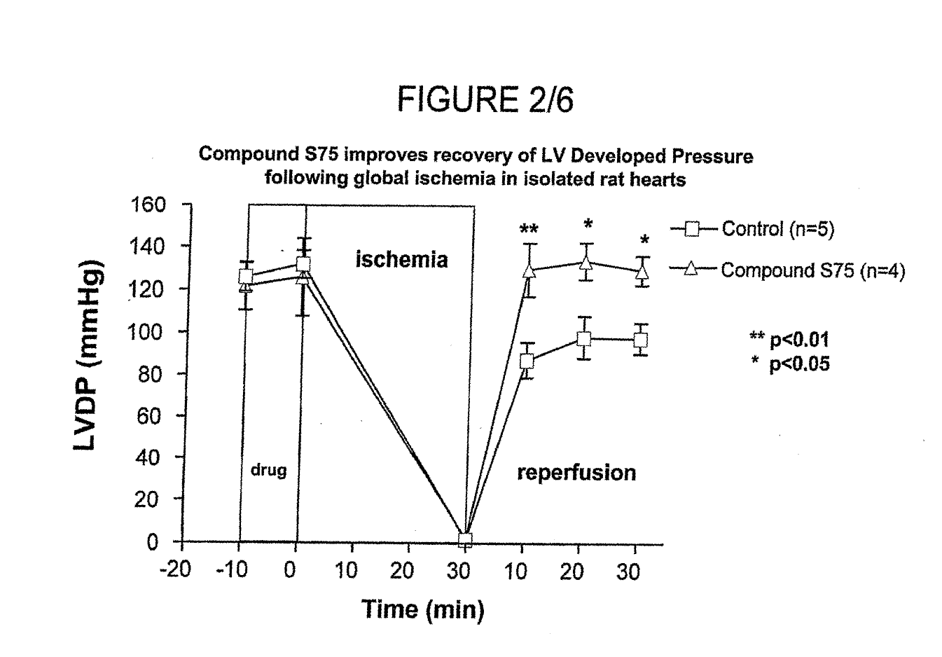

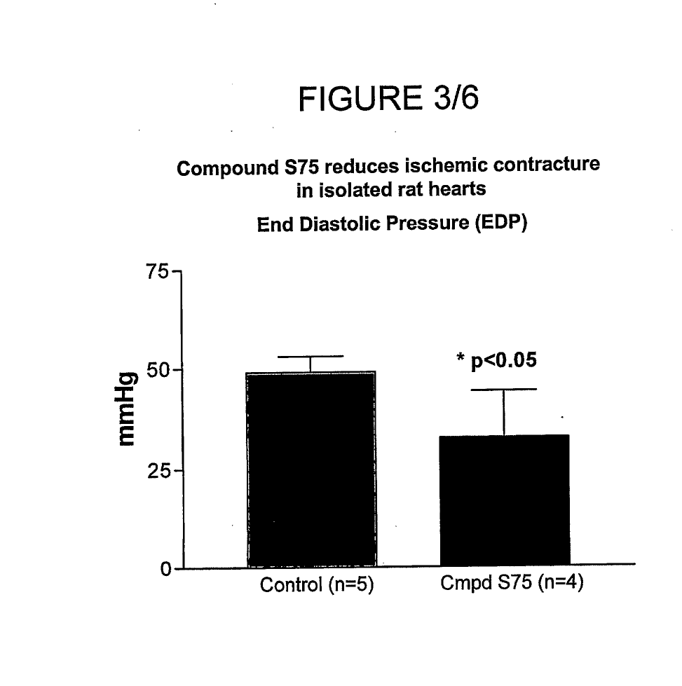

An example of a Compound of the Invention tested in this assay is shown in FIG. 2. In this example, Compound S75 at a concentration of 30 μM was found to provide protection against ischemia-reperfusion injury in isolated rat hearts as shown by a significant increase in left ventricle function after reperfusion compared to vehicle treatment. As shown in FIG. 2, the level of left ventricle function after reperfusion in Compound S75 treated hearts was comparable to the level before ischemia. In addition, as shown in FIG. 3, Compound S75 at a concentration of 30 μM was found to reduce ischemic contracture in isolated rat hearts as shown by a significant decrease in end diastolic pressure (EDP) compared to vehicle treatment.

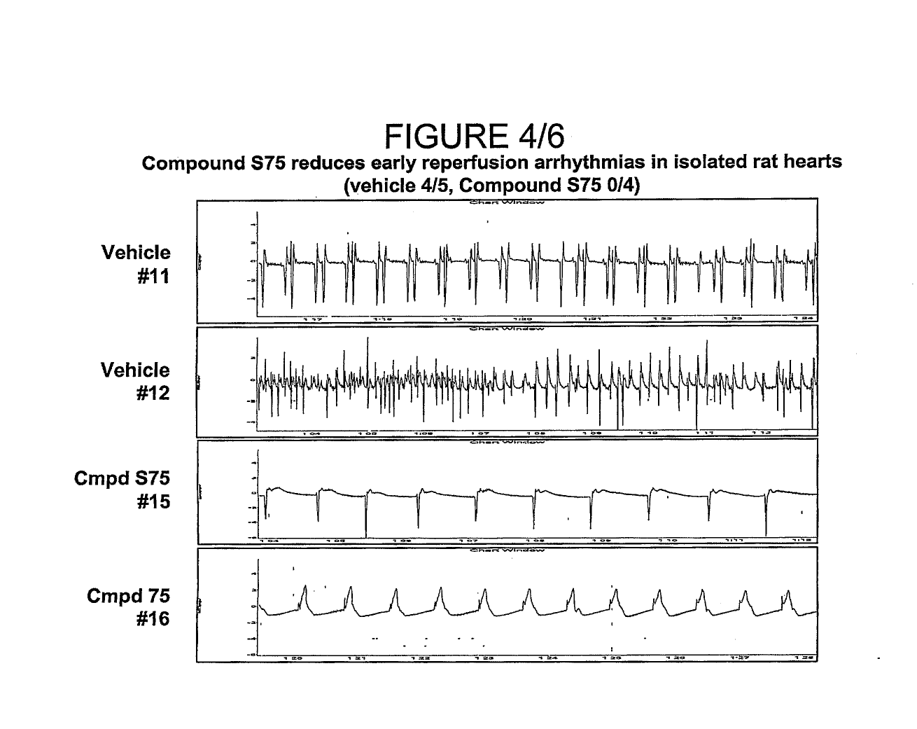

Epicardial electrogram recordings were also taken from the isolated rat hearts used in FIGS. 2 and 3. Briefly, silver wire electrodes were placed on the right atrium and the apex of the left ventricle, allowing an epicardial electrogram to be recorded. Premature ventricular contraction (PVC), ventricular tachycardia and ventricular fibrillation were common arrhythmias observed during reperfusion following 30 minutes of global ischemia. Hearts were considered positive for reperfusion arrhythmias if ventricular arrhythmias were sustained for greater than 30 seconds during the first 5 minutes of reperfusion. As shown in FIG. 4, early reperfusion arrhythmias were observed in vehicle treated isolated hearts but not in hearts treated Compound S75. In this experiment, none of the four hearts treated with Compound S75 showed early reperfusion arrhythmias while 4 of the 5 vehicle treated hearts showed early reperfusion arrhythmias.

### Example 5

### Measurement of Blood Pressure in Rats Exposed to Compound S75

Telemetry Studies:

Cardiac parameters were measured by small transmitting devices, (Data Sciences PhysioTel Telemetry devices), implanted in rats. The implanted transmitting devices were used to measure blood pressure in freely moving conscious animals. There are no external connections or tethering devices that can inhibit animal movement and induce unnecessary stress, which can affect the outcome of a study.

Transmitter Implantation:

This procedure was performed under modified aseptic conditions. Rats were anesthetized with Isoflurane gas that ranged in concentration from 1.5-2.0%. A cardiac telemetry device was implanted into the peritoneal cavity with a pressure sensing catheter situated no more than 2 cm inside the descending aorta. This was accomplished as follows: The rat was shaved and the incision site was prepared with an iodine solution. The rat was then placed on a heating pad to maintain a constant body temp of 38+/−0.5° C., and covered with a sterile drape. A 6 cm midline abdominal incision was made to provide access to the implantation area. Then the stomach muscle was cut with sharp scissors. The contents of the abdomen were exposed with retractors and the intestines were rearranged with wet gauze to expose the aorta. The aorta was separated from the vena cava. The aorta was then punctured just cranial to the aortic bifurcation with a bent 21 gauge needle. Immediately the pressure sensing catheter was inserted no more than 2 cm into the aorta. The site was thoroughly dried and 1-2 drops of Vet bond adhesive was applied. The site was checked to ensure there was no bleeding. Also, the signal from the transmitter was checked to verify that there was a sufficient signal from the transmitter. The gauze and retractors were then removed and the abdominal area was rinsed with sterile saline. These animals also have biopotential leads which were channeled through the stomach muscle with a sterile 16 gauge needle. Biopotential leads, which are used to measure an electrical signal generated by the contraction of the ventricles of the heart, were implanted into the muscle in order to obtain electrocardiogram (ECG) output, if desired. The skin incision sites for the biopotential leads and abdomen were closed with sterile incision staples. Antibiotic ointment was applied to the incision areas. Post operative antibiotics, (Sulfatrim-sulfamethoxazole+trimethoprim), were mixed with their drinking water, (20 mL/quart H₂O), for 5 days after surgery. The rats were monitored for 7 days to ensure proper recovery.

On the test day, injections with either vehicle or with test compound were administered via IP injection in volumes of ˜250 μl. Animals were monitored with a resolution of approximately one measurement/min for 60 minutes before injection of vehicle or compound and for about 120 minutes after injection.

FIG. 5 shows blood pressure readings obtained using the protocol described above. As expected, the vasoconstrictor angiotensin II (angII) resulted in a significant increase in blood pressure while the vasodilator sodium nitroprusside (snp) resulted in a significant decrease in blood pressure in treated rats. Treatment of rats with Compound S75 did not result in a significant change in blood pressure compared to the blood pressure readings recorded in these rats before treatment with the compound.

### Example 6

### Measurement of Inhibition on Angiotensin II Induced Vasoconstriction in Isolated Rat Aortic Rings with Compound S75

### Vasoconstriction Analysis—Preparation of Rat Aortic Rings

Sprague-Dawley rats (250-300 g) were euthanized via CO₂ chamber. The thoracic aorta was quickly harvested and placed in aerated (95% O₂ and 5% CO₂) modified Tyrode solution (in mM): NaCl 158.3, KCl 4, CaCl₂ 2, NaH₂PO₄.42, MgCl₂ 1.05, Glucose 5, NaHCO₃ 10, Ph 7.4. The excess adventitial tissue was carefully removed and the endothelium was sloughed off by rinsing with buffer via syringe. The vessel was then cut into 2-3 mm rings and mounted on wire stirrups connected to force transducers (World Precision Instruments, Myobath 4 channel system, FORT-10) to record changes in isometric force. The aortic rings were then placed in 15 mL organ baths filled with buffer at 37° C. with continuous aeration (95% O₂ and 5% CO₂)

The rings were left to equilibrate in buffer for 30 minutes. A resting tension of 4 g was applied and the rings were allowed to equilibrate for 1 hour. During the equilibration period, the buffer was changed every 30 minutes. After equilibration was complete, the aortic rings were tested for viability and maximal contraction response by addition of 50 mM KCl.

### Compound S75 Dose Response

Aortic rings were contracted with AngiotensinII (1 uM) Sigma, St Louis, Mo. and the isometric force recorded. The organ chamber was then washed out with buffer and left to equilibrate for 20 minutes. After equilibration, the buffer was replaced with appropriate concentration of Compound S75 and left for an additional 15 minutes. The aortic rings were then treated with the agonist, AngiotensinII (1 uM), and the change in isometric force was recorded. The process was repeated until a dose response curve was generated for Compound S75. The same doses of Compound S75 were tested against the vasoconstriction response to norepinephrine (NE) at 1 uM.

### Statistical Analysis

Data for each group (Compound S75 concentration) was from n=4 aortic rings. Change in aortic tension after AngiotensinII treatment was expressed as a percentage to 50 mM KCL in order to normalize tension for each aortic ring.

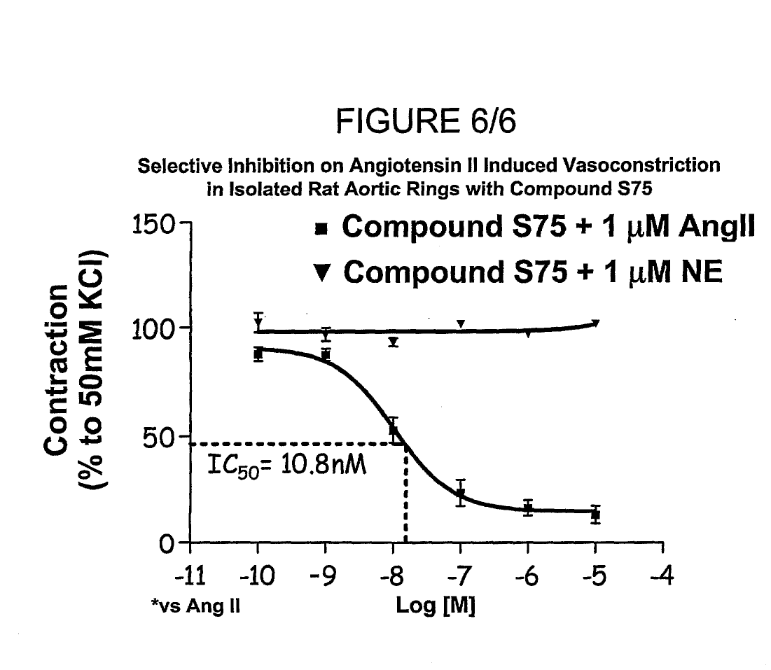

These data (FIG. 6) demonstrate that pretreatment with Compound S75 elicits a potent and selective inhibition on angiotensin II induced vasoconstriction in isolated rat aortic rings.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosures of which are incorporated herein by reference.

1. A compound of Formula (Ia): 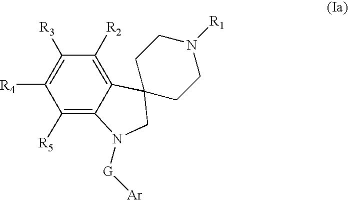
or a pharmaceutically acceptable salt, solvate or hydrate thereof;
wherein:
G is C(═O) or S(═O)₂;
R₁ is n-propyl optionally substituted with 1, 2, 3, 4, 5, 6, or 7 fluorine atoms;
R₂, R₃, R₄, and R₅ are each selected independently from the group consisting of H, C₁₋₆ acyl, C₁₋₆ acyloxy, C₂₋₆ alkenyl, C₁₋₆ alkoxy, C₁₋₆ alkyl, C₁₋₆ alkylamino, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylthiocarboxamide, C₁₋₆ alkylthioureyl, C₁₋₆ alkylureyl, amino, di-C₁₋₆-alkylamino, C₁₋₆ alkoxycarbonyl, carboxamide, carboxy, cyano, C₃₋₆ cycloalkyl, di-C₁₋₆-alkylcarboxamide, di-C₁₋₆-alkylsulfonamide, di-C₁₋₆-alkylthiocarboxamido, C₁₋₆ haloalkoxy, C₁₋₆ haloalkyl, halogen, C₁₋₆ haloalkylsulfinyl, C₁₋₆ haloalkylsulfonyl, C₁₋₆ haloalkylthio, heterocyclic, hydroxyl, nitro, sulfonamide, and thiol; and
Ar is aryl or heteroaryl each optionally substituted with 1, 2, 3, 4, 5, 6, or 7 substituents selected independently from the group consisting of C₁₋₆ acyl, C₁₋₆ acyloxy, C₂₋₆ alkenyl, C₁₋₆ alkoxy, C₁₋₆ alkyl, C₁₋₆ alkylamino, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylthiocarboxamide, C₁₋₆ alkylthioureyl, C₁₋₆ alkylureyl, amino, di-C₁₋₆-alkylamino, C₁₋₆ alkoxycarbonyl, carboxamide, carboxy, cyano, C₃₋₆ cycloalkyl, di-C₁₋₆-alkylcarboxamide, di-C₁₋₆-alkylsulfonamide, di-C₁₋₆-alkylthiocarboxamido, C₁₋₆ haloalkoxy, C₁₋₆ haloalkyl, halogen, C₁₋₆ haloalkylsulfinyl, C₁₋₆ haloalkylsulfonyl, C₁₋₆ haloalkylthio, heterocyclic, hydroxyl, nitro, sulfonamide, and thiol;
provided that said compound is not of the group consisting of:
1′-(propyl)-1,2-dihydro-5,7-dimethyl-1-(2-chloro-benzenesulfonyl)-spiro[3H-indole-3,4′-piperidine];
1′-(propyl)-1,2-dihydro-5-methyl-1-(2,3-difluoro-benzoyl)-spiro[3H-indole-3,4′-piperidine]; and
1′-(propyl)-1,2-dihydro-5-methyl-1-(2,6-difluoro-benzoyl)-spiro[3H-indole-3,4′-piperidine].

2. The compound according to claim 1, wherein G is C(═O).

3. The compound according to claim 1, wherein G is S(═O)₂.

4. The compound according to claim 1, wherein R₁ is n-propyl.

5. The compound according to claim 1, wherein R₁ is n-propyl substituted with 1, 2, 3, 4, 5, 6, or 7 fluorine atoms.

6. The compound according to claim 1, wherein R₁ is selected from the group consisting of CH₂CH₂CF₃, CH₂CF₂CF₃, CH₂CH₂CHF₂, —CH₂CF₂CHF₂, and CH₂CF₂CH₃.

7. The compound according to claim 1, wherein R₁ is —CH₂CH₂CF₃.

8. The compound according to claim 1, wherein R₂, R₃, R₄, and R₅ are each selected independently from the group consisting of H, C₁₋₆ alkoxy, C₁₋₆ alkyl, C₁₋₆ alkylcarboxamide, C₁₋₆ alkylsulfonamide, C₁₋₆ alkylsulfinyl, C₁₋₆ alkylsulfonyl, C₁₋₆ alkylthio, carboxamide, cyano, C₃₋₆ cycloalkyl, di-C₁₋₆-alkylcarboxamide, C₁₋₆ haloalkoxy, C₁₋₆ haloalkyl, halogen, C₁₋₆ haloalkylsulfinyl, C₁₋₆ haloalkylsulfonyl, C₁₋₆ haloalkylthio, hydroxyl, and sulfonamide.

9. The compound according to claim 1, wherein R₂, R₃, R₄, and R₅ are each selected independently from the group consisting of H, —OCH₃, CH₃, CH₂CH₃, CH(CH₃)₂, C(CH₃)₃, S(═O)₂NH₂, S(═O)₂NHCH₃, S(═O)CH₃, S(═O)₂CH₃, SCH₃, C(═O)NH₂, C≡N, C(═O)N(CH₃)₂, OCF₃, CF₃, CF₂CF₃, F, Cl, Br, I, S(═O)CF₃, —S(═O)₂CF₃, SCF₃, and OH.

10. The compound according to claim 1, wherein
R₂, R₄, and R₅ are each independently H, CH₃, or F; and
R₃ is selected from the group consisting of H, OCH₃, CH₂CH₃, CH(CH₃)₂, C(CH₃)₃, —S(═O)₂NH₂, S(═O)₂NHCH₃, S(═O)CH₃, S(═O)₂CH₃, SCH₃, C(═O)NH₂, C≡N, C(═O)N(CH₃)₂, —OCF₃, CF₃, CF₂CF₃, F, Cl, Br, I, S(═O)CF₃, S(═O)₂CF₃, SCF₃, and OH.

11. The compound according to claim 1, wherein
R₂, R₄, and R₅ are each H; and
R₃ is selected from the group consisting of H, OCH₃, CH(CH₃)₂, C(CH₃)₃, S(═O)₂NH₂, —S(═O)₂CH₃, C(═O)NH₂, C≡N, C(═O)N(CH₃)₂, CF₃, F, and Cl.

12. The compound according to claim 1, wherein
R₂, R₄, and R₅ are each H; and
R₃ is CH(CH₃)₂, C(CH₃)₃, CF₃, or Cl.

13. A compound according to claim 1, wherein Ar is phenyl or naphthyl each optionally substituted with 1, 2, 3, 4, 5, 6, or 7 substituents selected independently from the group consisting of halo, C₁₋₆ alkoxy, C₁₋₆ alkylsulfonyl, nitro, C₁₋₆ alkyl, carboxamide, cyano, C₁₋₆ haloalkyl, C₁₋₆ haloalkoxy, and sulfonamide.

14. A compound according to claim 1, wherein Ar is phenyl or naphthyl each optionally substituted with 1, 2, 3, 4, 5, 6, or 7 substituents selected independently from the group consisting of F, Cl, Br, OCH₃, OCH₂CH₃, —OCH(CH₃)₂, NO₂, CH₃, S(═O)₂CH₃, S(═O)₂NH₂, C≡N, CF₃, and OCF₃.

15. A compound according to claim 1, wherein Ar is selected from the group consisting of 2-chlorophenyl, 4-methoxyphenyl, 4-nitrophenyl, 3,4-difluorophenyl, 2-fluorophenyl, 4-chlorophenyl, 3-dichloromethylphenyl, 3,5-bis-trifluoromethylphenyl, 2,4-difluorophenyl, 2-methoxyphenyl, 3,5-dimethoxyphenyl, 3,4,5-trimethoxyphenyl, 3-fluorophenyl, 4-fluorophenyl, 2,3-difluorophenyl, 2,5-difluorophenyl, 3-trifluoromethyl-4-fluorophenyl, 3-trifluoromethylphenyl, phenyl, 3-nitrophenyl, 3-nitro-4-methylphenyl, naphthalen-1-yl, naphthalen-2-yl, 4-trifluoromethylphenyl, 3-chlorophenyl, 3,5-dichlorophenyl, 2,4-dichlorophenyl, 2,6-difluorophenyl, 2,6-dichlorophenyl, 2,6-bis-trifluoromethylphenyl, 2-chloro-6-fluoro-phenyl, and 2,3-dichlorophenyl.

16. A compound according to claim 1, wherein Ar is heteroaryl optionally substituted with 1, 2, 3, 4, or 5 substituents selected independently from the group consisting of halo, C₁₋₆ alkoxy, C₁₋₆ alkylsulfonyl, nitro, C₁₋₆ alkyl, carboxamide, cyano, C₁₋₆ haloalkyl, C₁₋₆ haloalkoxy, and sulfonamide.

17. A compound according to claim 1, wherein Ar is thienyl, pyridinyl, furanyl, pyrazolyl, benzofuranyl, benzothienyl, or naphthyridinyl each optionally substituted with 1, 2, 3, 4, or 5 substituents selected independently from the group consisting of halo, C₁₋₆ alkoxy, nitro, C₁₋₆ alkyl, and C₁₋₆ haloalkyl.

18. A compound according to claim 1, wherein Ar is thienyl, pyridinyl, furanyl, pyrazolyl, benzofuranyl, benzothiophenyl, or naphthyridinyl each optionally substituted with 1, 2, 3, 4, or 5 substituents selected independently from the group consisting of F, Cl, Br, OCH₃, OCH₂CH₃, OCH(CH₃)₂, NO₂, CH₃, and CF₃.

19. A compound according to claim 1, wherein Ar is selected from the group consisting of 3-chloro-thiophen-2-yl, benzothiophen-2-yl, 2,5-dichloro-thiophen-3-yl, and 2-trifluoromethyl-[1,6]naphthyridin-3-yl.

20. A compound according to claim 1 having Formula (IIa): 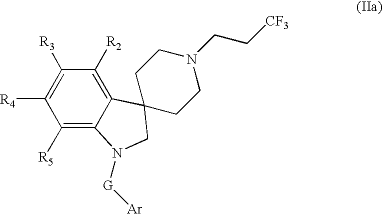
or a pharmaceutically acceptable salt, solvate or hydrate thereof;
wherein:
G is C(═O) or S(═O)₂;
R₂, R₄, and R₅ are each independently H, CH₃, or F;
R₃ is selected from the group consisting of H, OCH₃, CH₃, CH₂CH₃, CH(CH₃)₂, C(CH₃)₃, S(═O)₂NH₂, S(═O)₂NHCH₃, S(═O)CH₃, S(═O)₂CH₃, SCH₃, C(═O)NH₂, C≡N, C(═O)N(CH₃)₂, —OCF₃, CF₃, CF₂CF₃, F, Cl, Br, I, S(═O)CF₃, S(═O)₂CF₃, SCF₃, OH, and S(═O)₂NH₂; and
Ar is selected from the group consisting of 2-chlorophenyl, 4-methoxyphenyl, 4-nitrophenyl, 3,4-difluorophenyl, 2-fluorophenyl, 4-chlorophenyl, 3-dichloromethylphenyl, 3,5-bis-trifluoromethylphenyl, 2,4-difluorophenyl, 2-methoxyphenyl, 3,5-dimethoxyphenyl, 3,4,5-trimethoxyphenyl, 3-fluorophenyl, 4-fluorophenyl, 2,3-difluorophenyl, 2,5-difluorophenyl, 3-trifluoromethyl-4-fluorophenyl, 3-trifluoromethylphenyl, phenyl, 3-nitrophenyl, 3-nitro-4-methylphenyl, naphthalen-1-yl, naphthalen-2-yl, 4-trifluoromethylphenyl, 3-chlorophenyl, 3,5-dichlorophenyl, 2,4-dichlorophenyl, 2,6-difluorophenyl, 2,6-dichlorophenyl, 2,6-bis-trifluoromethylphenyl, 2-chloro-6-fluoro-phenyl, and 2,3-dichlorophenyl.

21. A compound according to claim 1 having Formula (IIa): 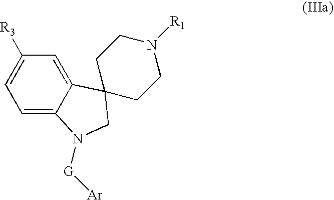
or a pharmaceutically acceptable salt, solvate or hydrate thereof;
wherein:
R₁ is n-propyl optionally substituted with fluorine;
G is C(═O) or S(═O)₂;
R₃ is CH(CH₃)₂ or C(CH₃)₃; and
Ar is selected from the group consisting of 2-chlorophenyl, 4-methoxyphenyl, 4-nitrophenyl, 3,4-difluorophenyl, 2-fluorophenyl, 4-chlorophenyl, 3-dichloromethylphenyl, 3,5-bis-trifluoromethylphenyl, 2,4-difluorophenyl, 2-methoxyphenyl, 3,5-dimethoxyphenyl, 3,4,5-trimethoxyphenyl, 3-fluorophenyl, 4-fluorophenyl, 2,3-difluorophenyl, 2,5-difluorophenyl, 3-trifluoromethyl-4-fluorophenyl, 3-trifluoromethylphenyl, phenyl, 3-nitrophenyl, 3-nitro-4-methylphenyl, naphthalen-1-yl, naphthalen-2-yl, 4-trifluoromethylphenyl, 3-chlorophenyl, 3,5-dichlorophenyl, 2,4-dichlorophenyl, 2,6-difluorophenyl, 2,6-dichlorophenyl, 2,6-bis-trifluoromethylphenyl, 2-chloro-6-fluoro-phenyl, and 2,3-dichlorophenyl.

22. A compound according to claim 1 having Formula (IVa): 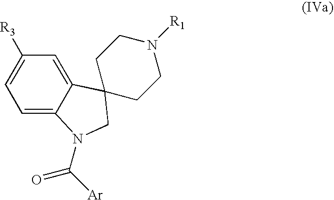
or a pharmaceutically acceptable salt, solvate or hydrate thereof;
R₁ is n-propyl optionally substituted with fluorine;
R₃ is CH(CH₃)₂ or C(CH₃)₃; and
Ar is selected from the group consisting of 2-chlorophenyl, 4-methoxyphenyl, 4-nitrophenyl, 3,4-difluorophenyl, 2-fluorophenyl, 4-chlorophenyl, 3-dichloromethylphenyl, 3,5-bis-trifluoromethylphenyl, 2,4-difluorophenyl, 2-methoxyphenyl, 3,5-dimethoxyphenyl, 3,4,5-trimethoxyphenyl, 3-fluorophenyl, 4-fluorophenyl, 2,3-difluorophenyl, 2,5-difluorophenyl, 3-trifluoromethyl-4-fluorophenyl, 3-trifluoromethylphenyl, phenyl, 3-nitrophenyl, 3-nitro-4-methylphenyl, naphthalen-1-yl, naphthalen-2-yl, 4-trifluoromethylphenyl, 3-chlorophenyl, 3,5-dichlorophenyl, 2,4-dichlorophenyl, 2,6-difluorophenyl, 2,6-dichlorophenyl, 2,6-bis-trifluoromethylphenyl, 2-chloro-6-fluoro-phenyl, and 2,3-dichlorophenyl.

23. A compound according to claim 1 having Formula (Va): 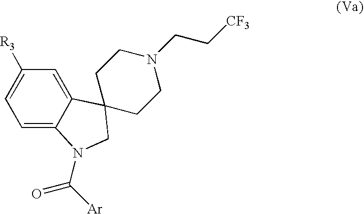
or a pharmaceutically acceptable salt, solvate or hydrate thereof;
wherein:
R₃ is CH(CH₃)₂, or C(CH₃)₃; and
Ar is phenyl or naphthyl each optionally substituted with 1, 2, 3, 4, 5, 6, or 7 substituents selected independently from the group consisting of F, Cl, Br, OCH₃, OCH₂CH₃, OCH(CH₃)₂, —NO₂, CH₃, S(═O)₂CH₃, S(═O)₂NH₂, C≡N, CF₃, and OCF₃.

24. A compound according to claim 1 having Formula (Va): 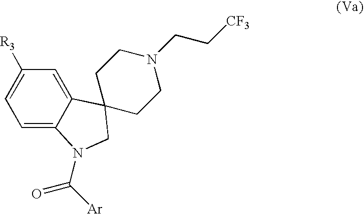
or a pharmaceutically acceptable salt, solvate or hydrate thereof;
wherein:
R₃ is CH(CH₃)₂, C(CH₃)₃, CF₃, or Cl; and
Ar is selected from the group consisting of 2-chlorophenyl, 2-fluorophenyl, 2,4-difluorophenyl, 2-methoxyphenyl, 2,3-difluorophenyl, 2,5-difluorophenyl, naphthalen-1-yl, 2,4-dichlorophenyl, 2,6-difluorophenyl, 2,6-dichlorophenyl, 2,6-bis-trifluoromethylphenyl, 2-chloro-6-fluoro-phenyl, and 2,3-dichlorophenyl.

25. A compound according to claim 1 having Formula (Va): 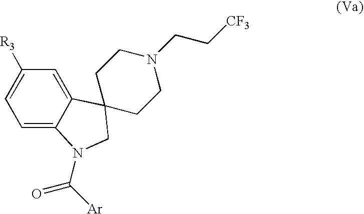
or a pharmaceutically acceptable salt, solvate or hydrate thereof;
wherein:
R₃ is CH(CH₃)₂, C(CH₃)₃, CF₃, or Cl; and
Ar is thienyl, pyridinyl, furanyl, pyrazolyl, benzofuranyl, benzothiophenyl, or naphthyridinyl each optionally substituted with 1, 2, 3, 4, or 5 substituents selected independently from the group consisting of F, Cl, Br, OCH₃, OCH₂CH₃, OCH(CH₃)₂, NO₂, CH₃, and CF₃.

26. A compound of claim 1 selected from the group consisting of:
1′-(3,3,3-trifluoro-propyl)-1,2-dihydro-5-isopropyl-1-(2,6-difluoro-benzoyl)-spiro[3H-indole-3,4′-piperidine];
1′-(3,3,3-trifluoro-propyl)-1,2-dihydro-5-tert-butyl-1-(2,6-difluoro-benzoyl)-spiro[3H-indole-3,4′-piperidine];
1′-(propyl)-1,2-dihydro-5-isopropyl-1-(2,6-difluoro-benzoyl)-spiro[3H-indole-3,4′-piperidine];
1′-(propyl)-1,2-dihydro-5-tert-butyl-1-(2,6-difluoro-benzoyl)-spiro[3H-indole-3,4′-piperidine];
1′-(propyl)-1,2-dihydro-5-methoxy-1-(2,6-difluoro-benzoyl)-spiro[3H-indole-3,4′-piperidine];
1′-(2,2,3,3,3-pentafluoro-propyl)-1,2-dihydro-5-isopropyl-1-(2,6-difluoro-benzoyl)-spiro[3H-indole-3,4′-piperidine];
1′-(2,2,3,3,3-pentafluoro-propyl)-1,2-dihydro-5-tert-butyl-1-(2,6-difluoro-benzoyl)-spiro[3H-indole-3,4′-piperidine];
1′-(3,3,3-trifluoro-propyl)-1,2-dihydro-5-chloro-1-(2,6-difluoro-benzoyl)-spiro[3H-indole-3,4′-piperidine];
1′-(3,3,3-trifluoro-propyl)-1,2-dihydro-5-trifluoromethyl-1-(2,6-difluoro-benzoyl)-spiro[3H-indole-3,4′-piperidine];
1′-(3,3,3-trifluoro-propyl)-1,2-dihydro-5-methanesulfonyl-1-(2,6-difluoro-benzoyl)-spiro[3H-indole-3,4′-piperidine];
1′-(3,3,3-trifluoro-propyl)-1,2-dihydro-5-sulfamoyl-1-(2,6-difluoro-benzoyl)-spiro[3H-indole-3,4′-piperidine];
1′-(3,3,3-trifluoro-propyl)-1,2-dihydro-5-cyano-1-(2,6-difluoro-benzoyl)-spiro[3H-indole-3,4′-piperidine];
1′-(3,3,3-trifluoro-propyl)-1,2-dihydro-5-carbamoyl-1-(2,6-difluoro-benzoyl)-spiro[3H-indole-3,4′-piperidine];
1′-(3,3,3-trifluoro-propyl)-1,2-dihydro-5-tert-butyl-1-(2,3-difluoro-benzoyl)-spiro[3H-indole-3,4′-piperidine];
1′-(3,3,3-trifluoro-propyl)-1,2-dihydro-5-tert-butyl-1-(2,6-dichloro-benzoyl)-spiro[3H-indole-3,4′-piperidine];
1′-(3,3,3-trifluoro-propyl)-1,2-dihydro-5-tert-butyl-1-(2,6-bis-trifluoromethyl-benzoyl)-spiro[3H-indole-3,4′-piperidine];
1′-(3,3,3-trifluoro-propyl)-1,2-dihydro-5-tert-butyl-1-(2,3-dichloro-benzoyl)-spiro[3H-indole-3,4′-piperidine];
1′-(3,3,3-trifluoro-propyl)-1,2-dihydro-5-tert-butyl-1-(3-chloro-thiophene-2-carbonyl)-spiro[3H-indole-3,4′-piperidine];
1′-(3,3,3-trifluoro-propyl)-1,2-dihydro-5-tert-butyl-1-(benzothiophene-2-carbonyl)-spiro[3H-indole-3,4′-piperidine];
1′-(3,3,3-trifluoro-propyl)-1,2-dihydro-5-tert-butyl-1-(naphthalene-1-carbonyl)-spiro[3H-indole-3,4′-piperidine];
1′-(3,3,3-trifluoro-propyl)-1,2-dihydro-5-tert-butyl-1-(2,5-dichloro-thiophene-3-sulfonyl)-spiro[3H-indole-3,4′-piperidine];
1′-(3,3,3-trifluoro-propyl)-1,2-dihydro-5-tert-butyl-1-(2,6-difluoro-benzenesulfonyl)-spiro[3H-indole-3,4′-piperidine];
1′-(3,3,3-trifluoro-propyl)-1,2-dihydro-5-tert-butyl-1-(2,3-difluoro-benzenesulfonyl)-spiro[3H-indole-3,4′-piperidine];
1′-(3,3,3-trifluoro-propyl)-1,2-dihydro-5-tert-butyl-1-(2,6-dichloro-benzenesulfonyl)-spiro[3H-indole-3,4′-piperidine];
1′-(3,3,3-trifluoro-propyl)-1,2-dihydro-5-tert-butyl-1-(2,6-bis-trifluoromethyl-benzenesulfonyl)-spiro[3H-indole-3,4′-piperidine];
1′-(3,3,3-trifluoro-propyl)-1,2-dihydro-5-tert-butyl-1-(2-trifluoromethyl-[1,6]naphthyridine-3-carbonyl)-spiro[3H-indole-3,4′-piperidine];
1′-(3,3,3-trifluoro-propyl)-1,2-dihydro-5-chloro-1-(2,3-difluoro-benzoyl)-spiro[3H-indole-3,4′-piperidine];
1′-(3,3,3-trifluoro-propyl)-1,2-dihydro-5-chloro-1-(2,6-dichloro-benzoyl)-spiro[3H-indole-3,4′-piperidine];
1′-(3,3,3-trifluoro-propyl)-1,2-dihydro-5-chloro-1-(2,6-bis-trifluoromethyl-benzoyl)-spiro[3H-indole-3,4′-piperidine];
1′-(3,3,3-trifluoro-propyl)-1,2-dihydro-5-chloro-1-(2,3-dichloro-benzoyl)-spiro[3H-indole-3,4′-piperidine];
1′-(3,3,3-trifluoro-propyl)-1,2-dihydro-5-trifluoromethyl-1-(2,3-difluoro-benzoyl)-spiro[3H-indole-3,4′-piperidine];
1′-(3,3,3-trifluoro-propyl)-1,2-dihydro-5-trifluoromethyl-1-(2,6-dichloro-benzoyl)-spiro[3H-indole-3,4′-piperidine];
1′-(3,3,3-trifluoro-propyl)-1,2-dihydro-5-trifluoromethyl-1-(2,6-bis-trifluoromethyl-benzoyl)-spiro[3H-indole-3,4′-piperidine]; and
1′-(3,3,3-trifluoro-propyl)-1,2-dihydro-5-trifluoromethyl-1-(2,3-dichloro-benzoyl)-spiro[3H-indole-3,4′-piperidine];
or a pharmaceutically acceptable salt, solvate or hydrate thereof.

27. A pharmaceutical composition comprising a compound according to claim 1 together with a pharmaceutically acceptable excipient.

28. A method for treating or preventing a vascular or cardiovascular disease or disorder comprising administering to a patient in need thereof an effective amount of a compound according to claim 1.

29. The method of claim 28, wherein said compound does not significantly increase blood pressure.

30. The method of claim 28, wherein the vascular or cardiovascular disorder is atherosclerosis, reperfusion injury, acute myocardial infarction, high blood pressure, primary or secondary hypertension, renal vascular hypertension, acute or chronic congestive heart failure, left ventricular hypertrophy, vascular hypertrophy, glaucoma, primary or secondary hyperaldosteronism, diabetic nephropathy, glomerulonephritis, scleroderma, glomerular sclerosis, renal failure, renal transplant therapy, diabetic retinopathy or migraine.

31. A method for treating or preventing a neurological disease or disorder comprising administering to a patient in need thereof an effective amount of a compound according to claim 1 or claim 26.

32. The method of claim 31, wherein the neurological disease or disorder is diabetic peripheral neuropathy, pain, stroke, cerebral ischemia or Parkinson's disease.

33. A method for treating or preventing a disease or disorder treatable or preventable by inhibiting Mas receptor function, comprising administering to a patient in need thereof an effective amount of a compound according to claim 1 or claim 26.

34. The method of claim 33, wherein the disease or disorder is a vascular or cardiovascular disease or disorder.

35. The method of claim 34, wherein the vascular or cardiovascular disease or disorder is atherosclerosis, reperfusion injury, acute myocardial infarction, high blood pressure, primary or secondary hypertension, renal vascular hypertension, acute or chronic congestive heart failure, left ventricular hypertrophy, vascular hypertrophy, glaucoma, primary or secondary hyperaldosteronism, diabetic nephropathy, glomerulonephritis, scleroderma, glomerular sclerosis, renal failure, renal transplant therapy, diabetic retinopathy or migraine.

36. The method of claim 33, wherein the disease or disorder is a neurological disease or disorder.

37. The method of claim 36, wherein the neurological disease or disorder is diabetic peripheral neuropathy, pain, stroke, cerebral ischemia or Parkinson's disease.

38.-44. (canceled)

45. A method of producing a pharmaceutical composition comprising admixing a compound according to claim 1 and a pharmaceutically acceptable vehicle or excipient.
